(19) United States
(12) Patent Application Publication
(10) Pub. No.: US 2025/0032538 A1
(43) Pub. Date: Jan. 30, 2025

(54) METHOD AND COMPOSITION FOR RESTORING HOMEOSTASIS, TREATING, AND PREVENTING DEGENERATIVE DISEASES, ACHIEVING ANTI-AGING EFFECT

(71) Applicant: Vector Vitale IP LLC, North Miami Beach, FL (US)

(72) Inventor: Peter NOVAK, Sunny Isles Beach, FL (US)

(73) Assignee: Vector Vitale IP LLC, North Miami Beach, FL (US)

(21) Appl. No.: 18/914,964

(22) Filed: Oct. 14, 2024

Related U.S. Application Data
(63) Continuation of application No. PCT/US24/39035, filed on Jul. 22, 2024.
(60) Provisional application No. 63/529,441, filed on Jul. 28, 2023.

(51) Int. Cl. A61K 33/30; A61K 33/00; A61K 33/04; A61K 33/06; A61K 33/24; A61K 33/26; A61K 33/36

(52) U.S. Cl. CPC A61K 33/30; A61K 33/00; A61K 33/04; A61K 33/06; A61K 33/24; A61K 33/26; A61K 33/36

(57) ABSTRACT

Disclosed are methods to restore homeostasis in a subject in need thereof; methods of treating or preventing a degenerative disease in a subject in need thereof; and methods of achieving anti-aging effect in a subject in need thereof. The disclosed methods establishes or rebuilds a homeostatic balance between electric and magnetic components of differential and integral characteristics of electromagnetic field in cells of said subject. The methods comprise administering to said subject a therapeutically effective amount of a pharmaceutical composition comprising various essential chemical elements and additive chemical elements specific to each essential element.

### TECHNICAL FIELD

This disclosure relates to anti-aging and preventing and treating degenerative diseases.

### BACKGROUND

Modern medicine cannot provide a complete cure for degenerative diseases, and therefore its main goal is to transfer the acute phase of the disease to chronic phase to prolong the life of patients. The problem is complicated because both degenerative disease and aging are accompanied by a sharp or gradual disruption of negative feedback or homeostasis at all levels of a living organism, starting at the cellular level. This greatly complicates both understanding the nature of the disease and the development of effective treatment methods.

For the successful treatment of degenerative diseases and the simultaneous prevention of aging, it is necessary to restore the stability of a living organism at the levels of cells, tissues, organs, and the body as a whole. The solution to this problem requires not only the restoration of negative feedback, but also the provision of minimal energy losses during all physiological processes and chemical reactions in the live cell. The lack of solution to the problems associated with the treatment of degenerative diseases and aging is because in fundamental science the concept of negative feedback is absent in the model of atom and in the theory of chemical bonds formation. Therefore, it is not surprising that the concept of negative feedback is absent in case of complex hierarchical system, an example of which is the human body. It is necessary to understand why the existence of such systems is possible in principle and how its stability must be ensured in time. The existing ideas about the emergence of life on our planet, as well as the evolutionary model of the development of life forms, do not give answers to this question.

Life expectancy has increased significantly over past hundred years mainly due to the use of antibiotics and improved quality of life. However, as population ages, the likelihood of oncological, cardiovascular, metabolic, and neurodegenerative disorders increases. Therefore, there is a huge unmet demand for new methods and therapeutical compositions to prevent and treat degenerative diseases, increase stability of human organism and slow down aging.

### SUMMARY

In one aspect, this disclosure provides a method to restore homeostasis in a subject in need thereof by establishing or rebuilding a homeostatic balance between electric and magnetic components of differential and integral characteristics of electromagnetic field in cells of said subject, comprising administering to said subject a therapeutically effective amount of a pharmaceutical composition comprising various essential chemical elements and additive chemical elements specific to each essential chemical element.

In another aspect, this disclosure provides a method of treating or preventing a degenerative disease in a subject in need thereof by establishing or rebuilding a homeostatic balance between electric and magnetic components of differential and integral characteristics of electromagnetic field in cells of said subject, comprising administering to said subject a therapeutically effective amount of a pharmaceutical composition comprising various essential chemical elements and additive elements specific to each essential element.

In yet another aspect, this disclosure provides a method of achieving anti-aging effect in a subject in need thereof comprising establishing or rebuilding a homeostatic balance between electric and magnetic components of differential and integral characteristics of electromagnetic field in cells of said subject, by administering to said subject a therapeutically effective amount of a pharmaceutical composition comprising various essential chemical elements and additive elements specific to each essential element.

In another aspect, this disclosure provides a pharmaceutical composition comprising therapeutically effective amounts of various essential chemical elements and additive chemical elements specific to each essential chemical element in specific quantities for restoring homeostasis in a subject in need thereof, by establishing or rebuilding a homeostatic balance between electric and magnetic components of differential and integral characteristics of electromagnetic field in cells of said subject.

In another aspect, this disclosure provides a system for restoring homeostasis in a subject in need thereof, comprising a pharmaceutical composition comprising therapeutically effective amounts of various essential chemical elements and additive chemical elements specific to each essential chemical element in specific quantities to be administered to said subject, after administering to said subject said composition, said system establishes or rebuilds a homeostatic balance between electric and magnetic components of differential and integral characteristics of electromagnetic field in cells of said subject.

Numerous other aspects are provided in accordance with these and other aspects of the disclosed methods. Other features and aspects of the disclosed methods will become more fully apparent from the following detailed description and the appended claims.

### BRIEF DESCRIPTION OF THE DRAWINGS

FIG. 1 shows a scheme of self-oscillations of the vector function \vec{F}.

FIG. 2 shows a scheme of self-oscillations of the vector function of the polarization potential \vec{Π}.

FIG. 3 shows Curves of the ionization potentials.

FIG. 4 shows reduced scale of ionization potentials.

FIG. 5 shows dot diagram of the ionization energy.

FIG. 6 shows ionization energies of the isoelectronic series as a sequential series of parallel smooth curves/parabolas.

FIG. 7 shows ionization energies of the isoelectronic series as a sequential series of parallel smooth curves/parabolas on a larger scale.

FIG. 8 shows ionization and characteristics X-Ray radiation energies versus atomic number.

FIG. 9 shows ionization potentials versus number of protons according to Handbook of the physicochemical properties of the elements. Edited by G. V. Samsonov. IFI/Plenum, New York-Washington, 1968.

FIG. 10 shows dependence of the excess of neutrons over protons versus the sum of s- and p-electrons.

FIG. 11 shows dependence of the excess of neutrons over protons versus the sum of d- and f-electrons.

FIG. 12 shows mechanism of Degenerative Disease via Heavy Isotope Substitution Disrupting Proteostasis.

FIG. 13 shows cytotoxic/cytostatic action of natural zinc and Zn-64 compounds on HL-60 cells.

### DETAILED DESCRIPTION

As used herein, the word “a” or “plurality” before a noun represents one or more of the particular noun.

The word “comprise” or variations such as “comprises” or “comprising” will be understood to imply the inclusion of a stated integer or groups of integers but not the exclusion of any other integer or group of integers.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

It is further to be understood that the feature or features of one embodiment may generally be applied to other embodiments, even though not specifically described or illustrated in such other embodiments, unless expressly prohibited by this disclosure or the nature of the relevant embodiments. Likewise, compositions and methods described herein can include any combination of features and/or steps described herein not inconsistent with the objectives of the present disclosure. Numerous modifications and/or adaptations of the compositions and methods described herein will be readily apparent to those skilled in the art without departing from the present subject matter.

All ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9. Any one or more individual members of a range or group disclosed herein may be excluded from a claim of this disclosure. The disclosure illustratively described herein suitably may be practiced in the absence of any element or elements, limitation or limitations which is not specifically disclosed herein. All ranges disclosed herein are also to be considered to include the end points of the range, unless expressly stated otherwise. For example, a range of “between 5 and 10” or “5 to 10” or “5-10” should be considered to include the end points 5 and 10.

When a Markush group or other grouping is used herein, all individual members of the group and all combinations and possible sub-combinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified may be employed in the practice of the disclosure without resort to undue experimentation. All art-known functional equivalents, of any such methods, device elements, and materials are intended to be included in this disclosure.

Unless defined otherwise, all technical and scientific terms used herein have the same meaning as commonly understood by one of ordinary skill in the art to which the disclosure pertains. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Degenerative Diseases and Aging

Modern medicine cannot provide a complete cure for degenerative diseases, and therefore its main goal is to transfer the acute phase of the disease to chronic to prolong the life of patients. The problem is complicated because both degenerative disease and aging are accompanied by a sharp or gradual disruption of negative feedback or homeostasis at all levels of a living organism, starting at the cellular level. This greatly complicates both understanding the nature of the disease and the development of effective treatment methods.

The mechanism of the onset of most degenerative diseases remains unknown. At best, thanks to the established correlation between certain disease and defects in the DNA structure, pathogenesis is explained by DNA mutations and risk factors that may cause them. Since current medicine is unable to eliminate DNA defects, treatment methods are mainly aimed at blocking corresponding mutant proteins and associated pathways. In many cases, diseases are associated not with one but with several mutations in DNA. However, because inhibitors have strong harmful side effects as a therapeutic target, in most cases it is necessary to choose the most overexpressed mutant protein only. The logic of using inhibitors and, in some cases, activators, is very simple. If certain molecular signatures are overexpressed in pathology affected cell, then they are needed for their vital activity. Therefore, the corresponding inhibitors must either slow down activity or just kill these cells. This approach is quite often statistically justified for the lack of a better way. At the same time, the inhibitor itself and the mutant proteins left unaddressed will inevitably destroy homeostasis and not only in pathology cells, but also in perfectly healthy ones. The result of such a situation is quite predictable: “If homeostasis is successful, life continues; if unsuccessful, disaster or death ensues”. https://www.britannica.com/science/homeostasis. Accumulation of DNA mutations with time seems inevitable especially in non-replicating cells where DNA repair pathways are ineffective. In replicating cells problems arise due to mutations in DNA repair genes.

It is generally accepted that aging is the most powerful and universal risk factor for all types of living organisms. Disruption of homeostasis is certainly an integral part of aging. So, it makes sense to consider degenerative disease as an acute local aging. Regardless of the type of the degenerative disease or the rate of aging due to the destruction of homeostasis/negative feedback, the symptoms and mechanism of violation of the stability of a living self-sustained system characterized by partial or complete loss of functionality are the results.

Thus, for the successful treatment of degenerative diseases and the simultaneous prevention of aging, it is necessary to restore the stability of a living organism at the levels of cells, tissues, organs, and the body as a whole. The solution to this problem requires not only the restoration of negative feedback, but also the provision of minimal energy losses during all physiological processes and chemical reactions in the live cell. The lack of solution to the problems associated with the treatment of degenerative diseases and aging is because in fundamental science the concept of negative feedback is absent in the model of atom and in the theory of chemical bonds formation. Therefore, it is not surprising that the concept of negative feedback is absent in case of complex hierarchical system, an example of which is the human body. It is necessary to understand why the existence of such systems is possible in principle and how its stability must be ensured in time. The existing ideas about the emergence of life on our planet, as well as the evolutionary model of the development of life forms, do not give answers to this question.

Life expectancy has increased significantly over past hundred years mainly due to the use of antibiotics and improved quality of life. However, as population ages, the likelihood of oncological, cardiovascular, metabolic, and neurodegenerative disorders is increasing. Therefore, there is a huge unmet demand for new methods and therapeutical compositions to prevent and treat degenerative diseases, increase stability of human organism and slow down aging.

Plasma-dust experiments carried out on the International Space Station in a cryogenic discharge at microgravity have demonstrated that “crystal structures” like DNA and made of cerium dioxide or plastic particles can be self-organized because of simultaneous action of interconnected generators of electric oscillations having frequencies between 1 and 255 Hz with amplitudes up to +/−55V and even different phases at temperatures below 4 K and quasi-zero gravitation. Changes in the temperature, parameters of oscillations or gravity led not only to the appearance of defects but to complete degeneration of the crystal DNA-like structure. V N Tsytovich et al., New Journal of Physics 9 (2007) 263; H M Thomas et al., New Journal of Physics, Volume 10, March 2008. In other words, the parameters of scalar and vector fields in this case were decisive for the stability of the self-organized system regardless of the chemical composition of the dust particles.

The above experiment confirms that an understanding of the general principles of the self-organization of inanimate or living matter is a necessary condition for progress in technology and medicine. To solve a problem, one must understand its nature.

It is pertinent to note here that biological molecules assemble in a very short time (from a few picoseconds to microseconds). Then more complex structures are formed and always in a well-defined order. How do atoms, molecules and live cells know what to do and in what sequence? The plasma dust experiment suggests that the self-organization of matter is determined by the existing (electromagnetic, gravitational) and newly formed fields of a higher order. In this case, the atoms, molecules, and more complex structures will sequentially line up along the lines of force of such fields. The stability of these fields will be the key to the life and death of a complex hierarchical system. According to the philosopher Emmanuel Kant, “Each subject has as much science as much there is mathematics in it”. Shown herein is why the formation of stable systems is possible in general and what needs to be done so that live systems retain their functional capabilities for as long as possible.

### General Model of the Self-Sustained Oscillating System

A scalar field is a function of a point in a space, the values of which are real numbers. For example, the density of a body in its different points is a scalar field. Geometrically, a scalar field is represented by the concepts of contour lines and level surfaces. The contour line is a line on which the function defining the field has a constant value. For example, isotherms are level curves of a temperature scalar field. The level surface is a surface on which the function defining the field has a constant value. For example, the surface of a conductor at electrical equilibrium has one and the same value of the electric potential at all its points and therefore is a surface level. A vector field is a function of a point defined on a subset of the three-dimensional Euclidean space, the values of which are vectors applied at the points of this subset. For example, the gravitational forces acting on a material point from the side of a mass distributed in space form a gravitational field which is a vector field. Mathematical Handbook for scientists and engineers. Definitions, Theorems and formulas for reference and review. G. A. Korn, T. M. Korn, McGraw-Hill Book Company, New York, San Francisco, Toronto, London, Sydney, 1968.

Geometrically, a vector field is represented by the concept of a vector line (streamline). A vector line (streamline) is a line at each point of which its tangent has a direction of vector of a vector field at that point. For example, circles formed by iron filings around a direct current carrying linear conductor are streamlines. Mathematical Handbook for scientists and engineers. Definitions, Theorems and formulas for reference and review. G. A. Korn, T. M. Korn, McGraw-Hill Book Company, New York, San Francisco, Toronto, London, Sydney, 1968.

For the convenience of working with scalar and vector fields, a linear Hamiltonian operator \vec{V} is used which takes the following form in Cartesian orthogonal coordinates:

$$\vec{∇} ≡ \frac{\partial }{\partial x} \vec{i} + \frac{\partial }{\partial y} \vec{j} + \frac{\partial }{\partial z} \vec{k}$$

Mathematical Handbook for scientists and engineers. Definitions, Theorems and formulas for reference and review. G. A. Korn, T. M. Korn, McGraw-Hill Book Company, New York, San Francisco, Toronto, London, Sydney, 1968.

Applying the Hamiltonian operator to a scalar field Φ(x,y,z), the result is the gradient of the scalar field, which is a vector field. The gradient of a scalar field is a vector directed along the normal to the surface level at a given point in the direction of increasing the scalar field at this point. The gradient of a scalar field Φ(x,y,z) is denoted by \vec{∇}Φ and takes the following form in Cartesian orthogonal coordinates:

$$\vec{∇} Φ = \frac{\partial Φ}{\partial x} \vec{i} + \frac{\partial Φ}{\partial y} \vec{j} + \frac{\partial Φ}{\partial z} \vec{k}$$

Mathematical Handbook for scientists and engineers. Definitions, Theorems and formulas for reference and review. G. A. Korn, T. M. Korn, McGraw-Hill Book Company, New York, San Francisco, Toronto, London, Sydney, 1968.

Applying the Hamiltonian operator to a vector \vec{F}(x,y,z), taking in its action as a non-commutative operation of scalar multiplication of two vectors \vec{∇}·\vec{F}, the result of this operation is called the divergence of a vector field. The divergence is a scalar field. In Cartesian coordinates, the divergence is:

$$\vec{∇} \cdot \vec{F} = \frac{\partial {F}_{x}}{\partial x} + \frac{\partial {F}_{y}}{\partial y} + \frac{\partial {F}_{z}}{\partial z}$$

Mathematical Handbook for scientists and engineers. Definitions, Theorems and formulas for reference and review. G. A. Korn, T. M. Korn, McGraw-Hill Book Company, New York, San Francisco, Toronto, London, Sydney, 1968.

Applying the Hamiltonian operator to a vector field \vec{F}(x,y,z), taking in its action as a non-commutative operation of vector multiplication of two vectors \vec{∇}×\vec{F}, the result of this operation is called a Curl of a vector field. The Curl is a vector field. In the Cartesian coordinates, the Curl is:

$$\vec{∇} \cdot \vec{F} = ( \frac{\partial {F}_{z}}{\partial y} - \frac{\partial {F}_{y}}{\partial y} ) \vec{i} + ( \frac{\partial {F}_{x}}{\partial z} - \frac{\partial {F}_{z}}{\partial x} ) \vec{j} + ( \frac{\partial {F}_{y}}{\partial x} - \frac{\partial {F}_{x}}{\partial y} ) \vec{k}$$

A scalar product of the Hamiltonian operator with itself is given by the Laplace operator \vec{∇}²:

$${\vec{∇}}^{2} ≡ \vec{∇} \cdot \vec{∇} = \frac{{\partial }^{2}}{\partial {x}^{2}} + \frac{{\partial }^{2}}{\partial {y}^{2}} + \frac{{\partial }^{2}}{\partial {z}^{2}}$$

The Laplace operator can be applied both to scalar and vector functions:

$${\vec{∇}}^{2} Φ ≡ \frac{{\partial }^{2} Φ}{\partial {x}^{2}} + \frac{{\partial }^{2} Φ}{\partial {y}^{2}} + \frac{{\partial }^{2} Φ}{\partial {z}^{2}}$$

$${\vec{∇}}^{2} \vec{F} ≡ ( {\vec{∇}}^{2} {F}_{x} ) \vec{i} + ( {\vec{∇}}^{2} {F}_{y} ) \vec{j} + ( {\vec{∇}}^{2} {F}_{z} ) \vec{k}$$

H M Thomas et al., New Journal of Physics, Volume 10, March 2008

Note the following rules for re-application of the Hamiltonian \vec{∇}:

$$\vec{∇} \cdot ( \vec{∇} Φ ) = {\vec{∇}}^{2} Φ$$

$$\vec{∇} \times ( \vec{∇} Φ ) = 0$$

$$\vec{∇} ( \vec{∇} \cdot \vec{F} ) = {\vec{∇}}^{2} \vec{F} + \vec{∇} \times ( \vec{∇} \times \vec{F} )$$

$$\vec{∇} \times ( \vec{∇} \times \vec{F} ) = \vec{∇} ( \vec{∇} \cdot \vec{F} ) - {\vec{∇}}^{2} \vec{F}$$

$$\vec{∇} \cdot ( \vec{∇} \times \vec{F} ) = 0$$

The directional derivative dΦ/dξ of a scalar point function Φ(x,y,z) is the rate at which the function changes with respect to the amount of movement ξ of a point (x,y,z) in the chosen direction. Mathematical Handbook for scientists and engineers. Definitions, Theorems and formulas for reference and review. G. A. Korn, T. M. Korn, McGraw-Hill Book Company, New York, San Francisco, Toronto, London, Sydney, 1968.

If the direction is given by a unit vector \vec{U}=COS [αₓ]\vec{i}+cos [α_{y}]\vec{j}+cos [αₓ]\vec{k}, where cos [αₓ], cos [α_{y}], cos [α_{z}] are the direction cosines, then the derivative in this direction is:

$$\frac{d Φ}{d ξ} = cos [ {α}_{x} ] \frac{\partial Φ}{\partial x} + cos [ {α}_{y} ] \frac{\partial Φ}{\partial y} + cos [ {α}_{z} ] \frac{\partial Φ}{\partial z} = ( \vec{u} \cdot \vec{∇} ) Φ$$

The directional derivative d\vec{F}/dξ of a vector point function \vec{F}(x,y,z) is defined in a similar way:

$$\frac{d \vec{F}}{d ξ} = ( \vec{u} \cdot \vec{∇} ) {F}_{x} \vec{i} \to + ( \vec{u} \cdot \vec{∇} ) {F}_{y} \vec{j} + ( \vec{u} \cdot \vec{∇} ) {F}_{z} \vec{k} = ( \vec{u} \cdot \vec{∇} ) \vec{F}$$

H M Thomas et al., New Journal of Physics, Volume 10, March 2008

Take a unit normal \vec{N} vector to the surface level, which is defined by the following expression, as a direction vector:

$$\vec{N} = \frac{\vec{∇} Φ}{\vert \vec{∇} Φ \vert}$$

The directional derivative of the unit normal vector of a scalar point function Φ(x,y,z) is called the normal derivative and is signed as dΦ/dN:

$$\frac{d Φ}{dN} = ( \vec{N} \cdot \vec{∇} ) Φ = \vec{N} \cdot \vec{∇} Φ$$

The directional derivative of the unit normal vector of a vector point function \vec{F}(x,y,z) is called the normal derivative and is signed as d\vec{F}/dN:

$$\frac{d \vec{F}}{dN} = ( \vec{N} \cdot \vec{∇} ) {F}_{x} \vec{i} \to + ( \vec{N} \cdot \vec{∇} ) {F}_{y} \vec{j} + ( \vec{N} \cdot \vec{∇} ) {F}_{z} \vec{k} = ( \vec{N} \cdot \vec{∇} ) \vec{F}$$

Laplacian \vec{∇}²Φ describes disturbance (oscillation) of a scalar point function Φ(x,y,z).

Laplacian \vec{∇}²\vec{F} describes disturbance (oscillation) of a vector point function \vec{F}(x,y,z).

Disturbance (oscillation) \vec{∇}²\vec{F} of an arbitrary vector function is due to the double Curl \vec{∇}×(\vec{∇}×\vec{F}) and the flow intensity gradient \vec{∇}(\vec{∇}·\vec{F}). The relation between these characteristics can be written as:

$${\vec{∇}}^{2} \vec{F} + \vec{∇} \times ( \vec{∇} \times \vec{F} ) - \vec{∇} ( \vec{∇} \cdot \vec{F} ) = 0 \qquad \text{(1-1)}$$

Mathematical Encyclopedic Dictionary, Chief Ed. A. M. Prokhorov.-Moscow, Soviet Encyclopedia, 1988,-847p.

The divergence is found using the following expression (1-1):

$$\vec{∇} \cdot ( {\vec{∇}}^{2} \vec{F} ) + \vec{∇} \cdot ( \vec{∇} \times ( \vec{∇} \times \vec{F} ) ) - {\vec{∇}}^{2} ( \vec{∇} \cdot \vec{F} ) = 0$$

The divergence of a Curl is always zero. H M Thomas et al., New Journal of Physics, Volume 10, March 2008.

$$\vec{∇} \cdot ( \vec{∇} \times ( \vec{∇} \times \vec{F} ) ) = 0$$

The Laplace operator and the divergence operator commute with each other:

$$\vec{∇} \cdot ( {\vec{∇}}^{2} \vec{F} ) - {\vec{∇}}^{2} ( \vec{∇} \cdot \vec{F} ) = 0$$

Expression \vec{∇}·(\vec{∇}²\vec{F}) describes the intensity of the vector \vec{F} disturbance (oscillation) flow.

Expression \vec{∇}²(\vec{∇}·\vec{F}) describes the disturbance (oscillation) of the vector \vec{F} flow intensity.

Thus, for any vector function \vec{F}, the intensity of the disturbance (oscillation) flow \vec{∇}·(\vec{∇}²\vec{F}) leads to the disturbance (oscillation) of the flow intensity \vec{∇}²(\vec{∇}·\vec{F}):

$${\vec{∇}}^{2} ( \vec{∇} \cdot \vec{F} ) = \vec{∇} \cdot ( {\vec{∇}}^{2} \vec{F} ) \qquad 1-2$$

The Curl is found using the following expression (1-2):

$$\vec{∇} \times ( {\vec{∇}}^{2} \vec{F} ) + \vec{∇} \times ( \vec{∇} \times ( \vec{∇} \times \vec{F} ) ) - \vec{∇} \times ( \vec{∇} ( \vec{∇} \cdot \vec{F} ) ) = 0$$

The Curl of the gradient is always zero. H M Thomas et al., New Journal of Physics, Volume 10, March 2008.

$$\vec{∇} \times ( \vec{∇} ( \vec{∇} \cdot \vec{F} ) ) = 0$$

The Curl of disturbances of the vector function \vec{∇}×(\vec{∇}²\vec{F}) and a triple Curl of the vector function \vec{∇}×(\vec{∇}×\vec{F})) are found to be equal in magnitude and have opposite directions:

$$\vec{∇} \times ( {\vec{∇}}^{2} \vec{F} ) = - \vec{∇} \times ( \vec{∇} \times ( \vec{∇} \times \vec{F} ) ) \qquad 1-3$$

Now substitute the Curl of the vector function \vec{∇}×\vec{F} in the expression (1-1):

$${\vec{∇}}^{2} ( \vec{∇} \times \vec{F} ) + \vec{∇} \times ( \vec{∇} \times ( \vec{∇} \times \vec{F} ) ) - \vec{∇} ( \vec{∇} \cdot ( \vec{∇} \times \vec{F} ) ) = 0$$

The divergence of a Curl is always zero. H M Thomas et al., New Journal of Physics, Volume 10, March 2008.

$$\vec{∇} ( \vec{∇} \cdot ( \vec{∇} \times \vec{F} ) ) = 0$$

The disturbance of the Curl of the vector function \vec{∇}²(\vec{∇}×\vec{F}) and a triple Curl of the vector function \vec{∇}×(\vec{∇}×\vec{F})) are found to be equal in magnitude and have opposite directions:

$${\vec{∇}}^{2} ( \vec{∇} \times \vec{F} ) = - \vec{∇} \times ( \vec{∇} \times ( \vec{∇} \times \vec{F} ) ) \qquad 1 - 4$$

Comparing the expressions (1-3) and (1-4), the Laplace operator and the Curl operator are found to be commute with each other:

$$\vec{∇} \times ( {\vec{∇}}^{2} \vec{F} ) - {\vec{∇}}^{2} ( \vec{∇} \times \vec{F} ) = 0$$

Expression \vec{∇}×(\vec{∇}²\vec{F}) describes a Curl of disturbances of the vector function \vec{F}.

Expression \vec{∇}²(\vec{∇}×\vec{F}) describes the disturbance (oscillation) of the Curl of the vector \vec{F}.

Thus, for any vector function F a Curl of disturbances \vec{∇}×(\vec{∇}²\vec{F}) leads to the disturbance of the Curl \vec{∇}²(\vec{∇}×\vec{F}):

$${\vec{∇}}^{2} ( \vec{∇} \times \vec{F} ) = \vec{∇} \times ( {\vec{∇}}^{2} \vec{F} ) \qquad 1 - 5$$

According to the divergence theorem, the flow of a vector through a closed surface is equal to the volume integral of the divergence limited by this surface:

$$( \vec{∇} \cdot ( {\vec{∇}}^{2} \vec{F} ) ) dV = {∯}_{S} d \vec{S} \cdot ( {\vec{∇}}^{2} \vec{F} ) \qquad 2 - 1$$

Mathematical Handbook for scientists and engineers. Definitions, Theorems and formulas for reference and review. G. A. Korn, T. M. Korn, McGraw-Hill Book Company, New York, San Francisco, Toronto, London, Sydney, 1968.

According to the Curl theorem, the “spread” of a vector through a closed surface is equal to the volume integral of the Curl limited by this surface:

$$( \vec{∇} \times ( {\vec{∇}}^{2} \vec{F} ) ) dV = {∯}_{S} d \vec{S} \times ( {\vec{∇}}^{2} \vec{F} ) \qquad 2 - 2$$

Mathematical Handbook for scientists and engineers. Definitions, Theorems and formulas for reference and review. G. A. Korn, T. M. Korn, McGraw-Hill Book Company, New York, San Francisco, Toronto, London, Sydney, 1968.

Enter

$$\vec{N} = \frac{d \vec{S}}{\vert d \vec{S} \vert} ,$$

a normal to a closed surface S.

In the case of a closed surface, the volume integral of the Laplacian of a scalar function is equal to the surface integral of its normal derivative (Gauss's theorem). Mathematical Encyclopedic Dictionary, Chief Ed. A. M. Prokhorov.-Moscow, Soviet Encyclopedia, 1988,-847p.

$$( {\vec{∇}}^{2} ( \vec{∇} \cdot \vec{F} ) ) dV = {∯}_{S} dS ( \frac{d}{dN} ( \vec{∇} \cdot \vec{F} ) ) \qquad 2 - 3$$

Similarly, for the vector function, in the case of a closed surface, the volume integral of the Laplacian of the vector function is equal to the surface integral of its normal derivative:

$$( {\vec{∇}}^{2} ( \vec{∇} \times \vec{F} ) ) dV = {∯}_{S} dS ( \frac{d}{dN} ( \vec{∇} \times \vec{F} ) ) \qquad 2 - 4$$

Mathematical Handbook for scientists and engineers. Definitions, Theorems and formulas for reference and review. G. A. Korn, T. M. Korn, McGraw-Hill Book Company, New York, San Francisco, Toronto, London, Sydney, 1968.
Mathematical Encyclopedic Dictionary, Chief Ed. A. M. Prokhorov.-Moscow, Soviet Encyclopedia, 1988,-847p.

Integrate the left-hand side and the right-hand side the expression (1-2) by volume:

$$( {\vec{∇}}^{2} ( \vec{∇} \cdot \vec{F} ) ) dV = ( \vec{∇} \cdot ( {\vec{∇}}^{2} \vec{F} ) ) dV$$

Use the integral expressions (2-1) and (2-3):

$${∯}_{S} dS ( \frac{d}{dN} ( \vec{∇} \cdot \vec{F} ) ) = {∯}_{S} dS ( \vec{N} \cdot ( {\vec{∇}}^{2} \vec{F} ) ) \qquad 2 - 5$$

The normal derivative of the vector function flow intensity is equal to the normal Laplacian projection of this vector function.

$$\frac{d}{dN} ( \vec{∇} \cdot \vec{F} ) = \vec{N} \cdot ( {\vec{∇}}^{2} \vec{F} ) \qquad 2 - 6$$

Integrate the left-hand side and the right-hand side the expression (1-5) by volume:

$$( {\vec{∇}}^{2} ( \vec{∇} \times \vec{F} ) ) dV = ( \vec{∇} \times ( {\vec{∇}}^{2} \vec{F} ) ) dV$$

Use the integral expressions (2-2) and (2-4):

$${∯}_{S} dS ( \frac{d}{dN} ( \vec{∇} \times \vec{F} ) ) = {∯}_{S} dS ( \vec{N} \times ( {\vec{∇}}^{2} \vec{F} ) ) \qquad 2 - 7$$

The normal derivative of a Curl of the vector function is equal to the Laplacian transverse component of this vector function.

$$\frac{d}{dN} ( \vec{∇} \times \vec{F} ) = \vec{N} \times ( {\vec{∇}}^{2} \vec{F} ) \qquad 2 - 8$$

### Self-Oscillating System

Self-oscillations are continuous oscillations supported by external sources of energy, in a nonlinear dissipative system, the type and properties of which are determined by the system itself. Self-oscillations fundamentally differ from the rest of oscillatory processes in a dissipative system as they do not require any external periodic action to sustain the motion.

An arbitrary vector function \vec{F} is shown, within the region of definition, and generates its own self-oscillating system and is its source (integral component) in this sense. Primary disturbances (oscillations) of the vector function \vec{F} are described by the Helmholtz equation:

$${\vec{∇}}^{2} \vec{F} = \frac{1}{{v}^{2}} \frac{{\partial }^{2} \vec{F}}{\partial {t}^{2}}$$

Mathematical Encyclopedic Dictionary, Chief Ed. A. M. Prokhorov.-Moscow, Soviet Encyclopedia, 1988,-847p.
James Clerk Maxwell, A treatise on electricity and magnetism. Moscow, Science, 1989, ISBN 5-02-000042-6.

Primary disturbances generate two types of secondary disturbances (oscillations).

A potential component of the primary disturbances gives rise to the secondary disturbances (oscillations) the of the vector function \vec{F} flow intensity:

$${\vec{∇}}^{2} ( \vec{∇} \cdot \vec{F} ) = \vec{∇} \cdot ( {\vec{∇}}^{2} \vec{F} )$$

A curl (solenoidal) component of the primary disturbances gives rise to the secondary disturbances (oscillations) of the Curl of the vector function \vec{F}:

$${\vec{∇}}^{2} ( \vec{∇} \times \vec{F} ) = \vec{∇} \times ( {\vec{∇}}^{2} \vec{F} )$$

The integral connection of the primary and secondary disturbances (2-5) and (2-7) is as follows:

$${∯}_{S} dS \{ \frac{d}{dN} ( \vec{∇} \cdot \vec{F} ) - \vec{N} \cdot ( {\vec{∇}}^{2} \vec{F} ) \} = 0$$

$${∯}_{S} dS \{ \frac{d}{dN} ( \vec{∇} \times \vec{F} ) - \vec{N} \times ( {\vec{∇}}^{2} \vec{F} ) \} = 0$$

The existence of connection at an integral level led to a feedback action of the secondary disturbances on the primary disturbances. There are two types of feedback.
1. Feedback on the potential component—the normal derivative of the vector function flow intensity affects the normal projection of the primary disturbances (2-6):

$$\frac{d}{dN} ( \vec{∇} \cdot \vec{F} ) - \vec{N} \cdot ( {\vec{∇}}^{2} \vec{F} ) = 0$$

2. Feedback on the solenoidal (curl) component—the normal derivative of the Curl of the vector function affects the transverse component of the primary disturbances (2-8).

$$\frac{d}{dN} ( \vec{∇} \times \vec{F} ) - \vec{N} \times ( {\vec{∇}}^{2} \vec{F} ) = 0$$

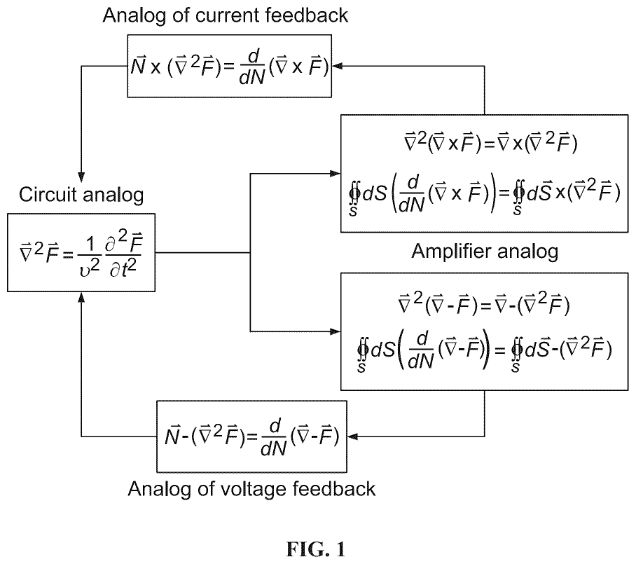

A self-sustained oscillation circuit results. FIG. 1.

Integral relationship of primary and secondary disturbances (oscillations) for an open surface S enclosed by a closed curve C

Use Stokes' theorem on circulation of the vector function \vec{F} and its vector analogue:

$${\int \int }_{S} d \vec{S} \cdot ( \vec{∇} \times \vec{F} ) = {∮}_{C} d \vec{r} \cdot \vec{F}$$

Mathematical Handbook for scientists and engineers. Definitions, Theorems and formulas for reference and review. G. A. Korn, T. M. Korn, McGraw-Hill Book Company, New York, San Francisco, Toronto, London, Sydney, 1968.
Mathematical Encyclopedic Dictionary, Chief Ed. A. M. Prokhorov.-Moscow, Soviet Encyclopedia, 1988,-847p.

$$\int {\int }_{S} ( d \vec{S} \times \vec{∇} ) \times \vec{F} = {∮}_{C} d \vec{r} \cdot \vec{F}$$

Here S is a simply connected open surface enclosed by a closed curve C.

Break down the integrand in the second surface integral:

$$( d \vec{S} \times \vec{∇} ) \times \vec{F} = ( d \vec{S} \cdot \vec{∇} ) \vec{F} + d \vec{S} \times ( \vec{∇} \times \vec{F} ) - d \vec{S} ( \vec{∇} \cdot \vec{F} )$$

Mathematical Handbook for scientists and engineers. Definitions, Theorems and formulas for reference and review. G. A. Korn, T. M. Korn, McGraw-Hill Book Company, New York, San Francisco, Toronto, London, Sydney, 1968.

Go to a unit normal vector

$$\vec{N} = \frac{d \vec{S}}{\vert d \vec{S} \vert}$$

of the open surface S:

$$\int {\int }_{S} dS \{ \vec{N} \cdot ( \vec{∇} \times \vec{F} ) \} = {∮}_{C} d \vec{r} \cdot \vec{F}$$

$$\int {\int }_{S} dS \{ \frac{d \vec{F}}{dN} + \vec{N} \times ( \vec{∇} \times \vec{F} ) - \vec{N} ( \vec{∇} \cdot \vec{F} ) \} = {∮}_{C} d \vec{r} \cdot \vec{F} \qquad 3 - 1$$

Substitute function \vec{F} for its Curl \vec{V}×\vec{F}:

$$\int {\int }_{S} dS \{ \vec{N} \cdot ( \vec{∇} \times ( \vec{∇} \times \vec{F} ) ) \} = {∮}_{C} d \vec{r} \cdot ( \vec{∇} \times \vec{F} )$$

$$\int {\int }_{S} dS \{ \frac{d}{dN} ( \vec{∇} \times \vec{F} ) + \vec{N} \times ( \vec{∇} \times ( \vec{∇} \times \vec{F} ) ) \} = {∮}_{C} d \vec{r} \times ( \vec{∇} \times \vec{F} )$$

Break down the double Curl and group the summands as follows:

$$\int {\int }_{S} dS \{ \frac{d}{dN} ( \vec{∇} \cdot \vec{F} ) - \vec{N} \cdot ( {\vec{∇}}^{2} \vec{F} ) \} = {∮}_{C} d \vec{r} \cdot ( \vec{∇} \times \vec{F} )$$

$$\int {\int }_{S} dS \{ \frac{d}{dN} ( \vec{∇} \times \vec{F} ) - \vec{N} \times ( {\vec{∇}}^{2} \vec{F} ) \} + \int {\int }_{S} d \vec{S} \times ( \vec{∇} ( \vec{∇} \cdot \vec{F} ) ) = {∮}_{C} d \vec{r} \times ( \vec{∇} \times \vec{F} )$$

A surface integral of the cross product of the area vector and the divergence of the gradient is expressed through the curvilinear integral:

$$\int {\int }_{S} d \vec{S} \times ( \vec{∇} ( \vec{∇} \cdot \vec{F} ) ) = {∮}_{C} d \vec{r} ( \vec{∇} \cdot \vec{F} )$$

Mathematical Handbook for scientists and engineers. Definitions, Theorems and formulas for reference and review. G. A. Korn, T. M. Korn, McGraw-Hill Book Company, New York, San Francisco, Toronto, London, Sydney, 1968.

In the case of a simply connected open surface S, which is enclosed by a closed curve C, the integral representation of the feedback between the primary and secondary disturbances \vec{∇}²\vec{F} and secondary disturbances of the flow intensity \vec{∇}²(\vec{∇}·\vec{F}) and secondary disturbances of a Curl \vec{∇}²(\vec{∇}×\vec{F}) is:

$$\int {\int }_{S} d S \{ \frac{d}{d N} ( \vec{∇} \cdot \vec{F} ) - \vec{N} \cdot ( {\vec{∇}}^{2} \vec{F} ) \} = {∮}_{C} d \vec{r} \cdot ( \vec{∇} \times \vec{F} ) \qquad 3 - 2$$

$$\int {\int }_{S} dS \{ \frac{d}{d N} ( \vec{∇} \times \vec{F} ) - \vec{N} \times ( {\vec{∇}}^{2} \vec{F} ) \} = {∮}_{C} d \vec{r} \times ( \vec{∇} \times \vec{F} ) - {∮}_{C} d \vec{r} ( \vec{∇} \cdot \vec{F} ) \qquad 3 - 3$$

The integral around the closed loop C on the right-hand side of the expression (3-2) describes circulation of a Curl \vec{∇}×\vec{F} of the vector function \vec{F}.

Express the vector line element d\vec{r} through the length of the arc ds and the unit tangent vector to the loop \vec{t} in the loop integrals on the right-hand side of the expression (3-3):

$$d \vec{r} = \frac{d \vec{r}}{ds} ds = \vec{t} ds$$

Mathematical Handbook for scientists and engineers. Definitions, Theorems and formulas for reference and review. G. A. Korn, T. M. Korn, McGraw-Hill Book Company, New York, San Francisco, Toronto, London, Sydney, 1968.
Mathematical Encyclopedic Dictionary, Chief Ed. A. M. Prokhorov.-Moscow, Soviet Encyclopedia, 1988,-847p.

The first loop integral describes the change in the local torque of the Curl \vec{t}×(\vec{∇}×\vec{F}) at the point of contact of the loop, when going around this loop C:

$${∮}_{C} d \vec{r} \times ( \vec{∇} \times \vec{F} ) = {∮}_{C} ds ( \vec{t} \times ( \vec{∇} \times \vec{F} ) )$$

The second loop integral describes the motion of the flow intensity \vec{t}(\vec{∇}·\vec{F}) in a closed curve C:

$${∮}_{C} d \vec{r} ( \vec{∇} \cdot \vec{F} ) = {∮}_{C} ds ( \vec{t} ( \vec{∇} \cdot \vec{F} ) )$$

Thus, for a simply connected open surface S enclosed by the closed curve C, the primary disturbances (oscillations) of the vector function \vec{F} relate to the translational and rotational motion of the secondary disturbances (oscillations).

For an arbitrary vector function \vec{F} two level surfaces S can be defined. The first level surface (signed as S₁) is defined by a constant value of the modulus of the vector function S₁=|\vec{F}|=const. The second surface (signed as S₂) is defined by a constant value of the flow intensity

$${S}_{2} = \vec{∇} \cdot \vec{F} = const .$$

The vector point function \vec{F}(x,y,z) is represented as a product of its module to a unit vector of the direction.

$$\vec{F} = a {\vec{i}}_{a} , where a = \vert \vec{F} \vert , \vert {\vec{i}}_{a} \vert = 1$$

Equations of the level surfaces S₁ and S₂ are:

$$\begin{matrix}{S}_{1} = a ( x , y , z ) = const , & {S}_{2} = \vec{∇} \cdot \vec{F} = const , \\ {\vec{N}}_{1} = \frac{\vec{∇} a}{\vert \vec{∇} a \vert} & {\vec{N}}_{2} = \frac{\vec{∇} ( \vec{∇} \cdot \vec{F} )}{\vert \vec{∇} ( \vec{∇} \cdot \vec{F} ) \vert}\end{matrix}$$

Where \vec{N}₁ and \vec{N}₂ are the normal unit vectors to the respective surface.

The level surfaces S₁ and S₂ exist independently of each other, and the main difference can be written as follows:

$${\int \int }_{{S}_{1}} d {\vec{S}}_{1} \times ( \vec{∇} ( \vec{∇} \cdot \vec{F} ) ) = {∮}_{{C}_{1}} d {\vec{r}}_{1} ( \vec{∇} \cdot \vec{F} )$$

$${\int \int }_{{S}_{2}} d {\vec{S}}_{2} \times ( \vec{∇} ( \vec{∇} \cdot \vec{F} ) ) = {\int \int }_{{S}_{2}} {dS}_{2} \{ {\vec{N}}_{2} \times {\vec{N}}_{2} \frac{d}{{dN}_{2}} ( \vec{∇} \cdot \vec{F} ) \} ≡ 0$$

The integral representation of the feedback between the primary disturbances \vec{∇}²\vec{F} and secondary disturbances of the flow intensity \vec{∇}²(\vec{∇}·\vec{F}) and secondary disturbances of the Curl \vec{∇}²(\vec{∇}×\vec{F}) for the surface S₁ enclosed by the closed loop C₁ is as follows:

$${\int \int }_{{S}_{1}} {dS}_{1} \{ \frac{d}{{dN}_{1}} ( \vec{∇} \cdot \vec{F} ) - {\vec{N}}_{1} \cdot ( {\vec{∇}}^{2} \vec{F} ) \} = {∮}_{{C}_{1}} d {\vec{r}}_{1} \cdot ( \vec{∇} \times \vec{F} )$$

$${\int \int }_{{S}_{1}} {dS}_{1} \{ \frac{d}{{dN}_{1}} ( \vec{∇} \times \vec{F} ) - {\vec{N}}_{1} \times ( {\vec{∇}}^{2} \vec{F} ) \} = {∮}_{{C}_{1}} d {\vec{r}}_{1} \times ( \vec{∇} \times \vec{F} ) - {∮}_{{C}_{1}} d {\vec{r}}_{1} ( \vec{∇} \cdot \vec{F} )$$

The integral representation of the feedback between the primary disturbances \vec{∇}²\vec{F} and secondary disturbances of the flow intensity \vec{∇}²(\vec{∇}·\vec{F}) and secondary disturbances of the Curl \vec{∇}²(\vec{∇}×\vec{F}) for the surface S₂ enclosed by the closed loop C₂ is as follows:

$${\int \int }_{{S}_{2}} {dS}_{2} \{ \frac{d}{{dN}_{2}} ( \vec{∇} \cdot \vec{F} ) - {\vec{N}}_{2} \cdot ( {\vec{∇}}^{2} \vec{F} ) \} = {∮}_{{C}_{2}} d {\vec{r}}_{2} \cdot ( \vec{∇} \times \vec{F} )$$

$${\int \int }_{{S}_{2}} {dS}_{2} \{ \frac{d}{{dN}_{2}} ( \vec{∇} \times \vec{F} ) - {\vec{N}}_{2} \times ( {\vec{∇}}^{2} \vec{F} ) \} = {∮}_{{C}_{2}} d {\vec{r}}_{2} \times ( \vec{∇} \times \vec{F} )$$

Example. Self-oscillations of the vector function \vec{F}(r,θ,φ) in spherical coordinates.

Assume that the module of the vector function depends only on the radius vector module:

$$\vec{F} ( r , θ , φ ) = a ( r ) \{ {\vec{i}}_{r} + {\vec{i}}_{θ} + {\vec{i}}_{φ} \}$$

$$\vec{∇} a ( r ) = \frac{\partial a ( r )}{\partial r} {\vec{i}}_{r} , \qquad {\vec{N}}_{1} = {\vec{i}}_{r}$$

1. The radial component α(r)\vec{i}ᵣ, is:

$$\begin{matrix}\vec{∇} \cdot ( a {\vec{i}}_{r} ) = \frac{2 a}{r} + \frac{\partial a}{\partial r} , & {\vec{N}}_{2} = {\vec{i}}_{r} & \vec{∇} \times ( a {\vec{i}}_{r} ) =\end{matrix} 0$$

There are two closed surfaces (in a simple case, these are two spheres of different sizes):

$${∯}_{{S}_{1}} {dS}_{1} \{ \frac{d}{d r} ( \vec{∇} \cdot ( a {\vec{i}}_{r} ) ) - {\vec{i}}_{r} \cdot ( {\vec{∇}}^{2} ( a {\vec{i}}_{r} ) ) \} ≡ 0 , here$$

$${S}_{1} = a ( r ) = const {∯}_{{S}_{2}} {dS}_{2} \{ \frac{d}{d r} ( \vec{∇} \cdot ( a {\vec{i}}_{r} ) ) - {\vec{i}}_{r} \cdot ( {\vec{∇}}^{2} ( a {\vec{i}}_{r} ) ) \} ≡ 0 , here$$

$${S}_{2} = \frac{2 a}{r} + \frac{\partial a}{\partial r} = const$$

2. The azimuthal component α(r)\vec{i}_{θ} is:

$$\begin{matrix}\vec{∇} \cdot ( a {\vec{i}}_{θ} ) = \frac{1}{r} \frac{cos [ θ ]}{sin [ θ ]} a & \vec{∇} \times ( a {\vec{i}}_{θ} ) =\end{matrix} {\vec{i}}_{φ} ( \frac{a}{r} + \frac{\partial a}{\partial r} )$$

The surfaces S₁ and S₂ are not closed. Openness S₁=α(r)=const means that the part of this surface is to be taken as enclosed by the closed loop C₁:

$${\int \int }_{{S}_{1}} {dS}_{1} \{ \frac{d}{{di}_{r}} ( \vec{∇} \cdot ( a {\vec{i}}_{θ} ) ) - {\vec{i}}_{r} \cdot ( {\vec{∇}}^{2} ( a {\vec{i}}_{θ} ) ) \} = {∮}_{{C}_{1}} d {\vec{r}}_{1} \cdot ( \vec{∇} \times ( a {\vec{i}}_{θ} ) ) \qquad 3 - 4$$

$${\int \int }_{{S}_{1}} {dS}_{1} \{ \frac{d}{{di}_{r}} ( \vec{∇} \times ( a {\vec{i}}_{θ} ) ) - {\vec{i}}_{r} \times ( {\vec{∇}}^{2} ( a {\vec{i}}_{θ} ) ) \} = {∮}_{{C}_{1}} d {\vec{r}}_{1} \times ( \vec{∇} \times ( a {\vec{i}}_{θ} ) ) - {∮}_{{C}_{1}} d {\vec{r}}_{1} ( \vec{∇} \cdot ( a {\vec{i}}_{θ} ) )$$

The expression (3-4) determines positioning of the surface S₂ relative to S₁. It follows from this that the integral connection between the primary and secondary disturbances causes the motion of the flow intensity \vec{t}₁(\vec{∇}·\vec{F}) in the closed curve C₁:

$${∮}_{{C}_{1}} d {\overline{r}}_{1} ( \vec{∇} \cdot ( a {\overline{i}}_{θ} ) ) = {∮}_{{C}_{1}} d {\overline{r}}_{1} \vec{t} ( \vec{∇} \cdot ( a {\vec{i}}_{θ} ) )$$

Consequently, the surface S₂ is located at the tangent point of the closed loop C₁. If the closed surface S₁ is a sphere and the closed loop C₁ is flat, the motion of the flow intensity \vec{t}₁(\vec{∇}·\vec{F}) in the closed curve C₁ is a motion in a circular orbit of a “spheroid”

$${S}_{2} = \frac{2 a}{r} + \frac{\partial a}{\partial r} = const .$$

From the expression (3-4) it also follows that at the tangent point of the closed loop C₁ there is a torque that rotates the “spheroid”

$${S}_{2} = \frac{2 a}{r} + \frac{\partial a}{\partial r} = const .$$

Assume that the closed loop C₁ is flat. In this case, the unit tangent vector to the loop \vec{t}₁ can be represented as follows:

$${\vec{t}}_{1} = \frac{d {\overline{r}}_{1}}{d {r}_{1}} = cos [ θ ] {\vec{i}}_{θ} + sin [ θ ] {\vec{i}}_{φ}$$

The azimuthal torque at the tangent point of the flat loop C₁ is:

$${\vec{t}}_{1} \times ( \vec{∇} \times ( a {\vec{i}}_{θ} ) ) = {\vec{i}}_{r} cos [ θ ] ( \frac{a}{r} + \frac{\partial a}{\partial r} )$$

The rotation axis of the “spheroid”

$${S}_{2} = \frac{2 a}{r} + \frac{\partial a}{\partial r} = const$$

lies in the ecliptic plane.

3. The tangential component α(r)\vec{i}_{φ} is:

$$\vec{∇} \cdot ( a {\vec{i}}_{φ} ) = 0 {\vec{∇}}^{} \times ( a {\vec{i}}_{φ} ) = {\vec{i}}_{z} \frac{a}{r sin [ θ ]} - {\vec{i}}_{θ} \frac{\partial a}{\partial r}$$

A tangential torque at the tangent point of the flat loop C₁ is:

$${\vec{t}}_{1} \times ( \vec{∇} \times ( a {\vec{i}}_{φ} ) ) = - {\vec{i}}_{φ} \frac{a {cos}^{2} [ θ ]}{r sin [ θ ]} + {\vec{i}}_{ρ} \frac{a cos [ θ ]}{r} + {\vec{i}}_{r} sin [ θ ] \frac{\partial a}{\partial r}$$

There is one more rotation axis of the “spheroid”

$${S}_{2} = \frac{2 a}{r} + \frac{\partial a}{\partial r} = const ,$$

and this axis may deviate from the plane of the ecliptic.

Thus, the self-oscillations of vector function \vec{F} in the spherical coordinates represent a motion in a circular orbit of the “spheroid” S₂=\vec{∇}·\vec{F}=const. And the spheroid has two axes of its own rotation that determine the intrinsic angular momentum of the “spheroid” based on the principle of the gyroscope.

The following conclusions follow from the above example:
1. The existence of the orbital motion and thus the orbital angular momentum means that the self-oscillating system of the vector function under consideration \vec{F}=α\vec{i}_{α} simultaneously defines the primary surface S₁=|\vec{F}|=const with the unit normal vector

$${\vec{N}}_{1} = \frac{\vec{∇} a}{\vert \vec{∇} a \vert}$$

and the flow intensity of the vector function \vec{∇}·\vec{F}.
2. The existence of the intrinsic angular momentum indicates that in the self-oscillating system of the vector function under consideration \vec{F}=α\vec{i}_{α} condition (1) is met and, moreover, the secondary surface S₂=\vec{∇}·\vec{F}=const is closed. If the secondary surface S₂=\vec{∇}·\vec{F}=const is not closed, then the intrinsic angular momentum is absent.

Thus, the presence of motion in a closed orbit (orbital motion) and/or the presence of intrinsic angular momentum in an object of the arbitrary nature mean that such object is a component (differential) part of a certain self-oscillating system.

In this regard, the physical meaning of any vector function is that, within its domain of definition, a vector function generates its own oscillating system, and in this sense, it is its source (integral component). At the same time, a vector function itself can be a differential component of a self-oscillating system of a higher level.

Vector functions form interconnected self-oscillating systems both at the differential level and at the integral level. Mathematically, there are no fundamental limitations on the lower or upper “limit”. In this sense, there is a hierarchy of differential and integral characteristics of the vector functions. Since the relationship between differential and integral components of the self-oscillating systems is described by the same operations of vector algebra, groups (sublevels) of characteristics formed in the image and likeness of a superior integrated group can be distinguished in this hierarchy.

It should be remembered that in accordance with the definition of self-oscillations, the life and death of a self-oscillating system depends on the presence or absence of an integral component.

### Self-Oscillations of the Vector Function of Polarization Potential \vec{Π}

The first derivative of the polarization potential \vec{Π} sets the vector potential \vec{A}. The divergence of the polarization potential \vec{Π} sets the scalar potential φ. In the International System of Units (SI), the relationship between these variables looks as follows:

$$\vec{A} = \frac{1}{{υ}^{2}} \frac{\partial \vec{\prod }}{\partial t} φ = - \vec{∇} \cdot \vec{\prod } \qquad 4-1$$

James Clerk Maxwell, A treatise on electricity and magnetism. Moscow, Science, 1989, ISBN 5-02-000042-6.
L. A. Vainstein. Electromagnetic waves.-Second ed., Moscow, Radio I Svyaz, 1988,-440p. ISBN 5-256-00064-0.

The vector potential \vec{A} and the scalar potential φ are connected between themselves by the Lorentz condition which in the International System of Units (SI) is:

$$\vec{∇} \cdot \vec{A} + \frac{1}{{υ}^{2}} \frac{\partial φ}{\partial t} + μ {μ}_{0} σφ = 0 \qquad 4-2$$

James Clerk Maxwell, A treatise on electricity and magnetism. Moscow, Science, 1989, ISBN 5-02-000042-6.
L. A. Vainstein. Electromagnetic waves.-Second ed., Moscow, Radio I Svyaz, 1988,-440p. ISBN 5-256-00064-0.

In its turn, the vector potential \vec{A} and the scalar potential φ define the vector functions of electric intensity \vec{E} and magnetic induction \vec{B}:

$$\vec{E} = - \vec{∇} φ - \frac{\partial \vec{A}}{\partial t} \vec{B} = \vec{∇} \times \vec{A} \qquad 4-3$$

James Clerk Maxwell, A treatise on electricity and magnetism. Moscow, Science, 1989, ISBN 5-02-000042-6.
L. A. Vainstein. Electromagnetic waves.-Second ed., Moscow, Radio I Svyaz, 1988,-440p. ISBN 5-256-00064-0.

The relationship between electric intensity \vec{E} and magnetic induction \vec{B} is described by Maxwell's differential equations:

$$\vec{∇} \times \vec{E} = - \frac{\partial \vec{B}}{\partial t} \vec{∇} \times \vec{B} = \frac{1}{{υ}^{2}} \frac{\partial \vec{E}}{\partial t} + μ {μ}_{0} \vec{j} \qquad 4-4$$

James Clerk Maxwell, A treatise on electricity and magnetism. Moscow, Science, 1989, ISBN 5-02-000042-6.

The divergence of electric intensity \vec{E} defines the volume charge density ρ. The divergence of magnetic induction \vec{B} is zero:

$$\begin{matrix}\vec{∇} \cdot \vec{E} = \frac{ρ}{{εε}_{0}} & \vec{∇} \cdot \vec{B} = 0\end{matrix} \qquad 4 - 5$$

James Clerk Maxwell, A treatise on electricity and magnetism. Moscow, Science, 1989, ISBN 5-02-000042-6.

Where σ is the specific conductivity of the medium, \vec{j} is the density of conduction current, ε, μ are the dielectric and magnetic permeabilities, ε₀, μ₀ are the dielectric and magnetic constants,

$$υ = \frac{c}{\sqrt{εμ}}$$

is the phase velocity,

$$c = \frac{1}{\sqrt{{ε}_{0} {μ}_{0}}}$$

is the speed of light in vacuum.

The relationship between the strength of electric field \vec{E} and the polarization potential \vec{Π} is of the form:

$$\vec{E} = \vec{∇} ( \vec{∇} \cdot \vec{Π} ) - \frac{1}{{υ}^{2}} \frac{{\partial }^{2} \vec{Π}}{\partial {t}^{2}} \qquad 4 - 6$$

Assume that the vector potential fluctuations \vec{Π} are described by the wave equation:

$${\vec{∇}}^{2} \vec{Π} = \frac{1}{{υ}^{2}} \frac{{\partial }^{2} \vec{Π}}{\partial {t}^{2}}$$

Mathematical Encyclopedic Dictionary, Chief Ed. A. M. Prokhorov.-Moscow, Soviet Encyclopedia, 1988,-847p.

In this approximation, the expression for the electric intensity \vec{E} will be:

$$\vec{E} = \vec{∇} ( \vec{∇} \cdot \vec{Π} ) - {\vec{∇}}^{2} \vec{Π} , or \vec{E} = \vec{∇} \times ( \vec{∇} \times \vec{Π} ) \qquad 4 - 7$$

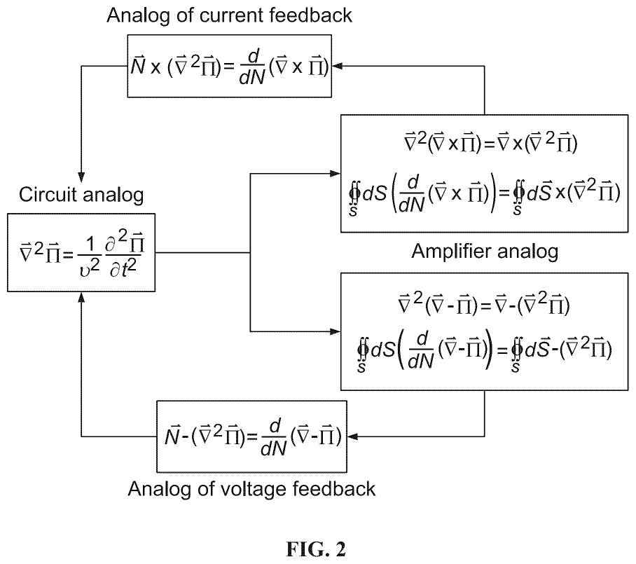

The scheme of self-oscillations of the vector function of the polarization potential \vec{Π} is presented on the FIG. 2.

A potential component of self-oscillations of the vector function \vec{Π} is:

$${\vec{∇}}^{2} ( \vec{∇} \cdot \vec{Π} ) = \vec{∇} \cdot ( {\vec{∇}}^{2} \vec{Π} )$$

Assume that the intensity of the primary mode flux of disturbances (oscillations) of the polarization potential \vec{Π} describes the density of the electric nucleus charge ρₘ:

$$\vec{∇} \cdot ( {\vec{∇}}^{2} \vec{Π} ) = \frac{{ρ}_{n}}{{εε}_{0}}$$

Also assume that the secondary mode of disturbances (oscillations) of the intensity flux describes the polarization potential \vec{Π} of the electric charge density of electron shells of an atom ρₑ:

$${\vec{∇}}^{2} ( \vec{∇} \cdot \vec{Π} ) = \frac{{ρ}_{e}}{{εε}_{0}}$$

Thus, the electric charge density in an atom as a localized and electrically neutral ρₑ=ρₙ self-oscillating system of the polarization potential \vec{Π} is described.

The vector function of the polarization potential \vec{Π} is represented as a product of its modulus Π=|\vec{Π}| on a unit guiding vector \vec{i}_{Π}:

\vec{Π}=Π\vec{i}_{Π}

Two level surfaces can be defined for the polarization potential function \vec{Π}. The first level surface (signed as S_{Π}) is defined by a constant value of the vector function modulus S_{Π}=|\vec{Π}|=const. The second surface (signed as S_{φ}) is defined by a constant value of the flow intensity S_{φ}=\vec{∇}·\vec{Π}=const.

If the level surfaces S_{Π} and S_{φ} are closed, there are two localization regions of self-oscillations. In the first case, a region of localization of self-oscillations is the volume V_{Π} limited by the simply connected closed surface S_{Π}. In the second case, a localization region of self-oscillations is the volume V_{φ} limited by the simply connected closed surface S_{φ}.

The vector elements of these surfaces d\vec{S}_{Π} and d\vec{S}_{φ} are of the form:

$$d {\vec{S}}_{Π} = {\vec{N}}_{Π} {dS}_{Π} , \qquad {\vec{N}}_{Π} = \frac{\vec{∇} Π}{\vert \vec{∇} Π \vert}$$

$$d {\vec{S}}_{φ} = {\vec{N}}_{φ} {dS}_{φ} , \qquad {\vec{N}}_{φ} = \frac{\vec{∇} φ}{\vert \vec{∇} φ \vert}$$

Here φ=−\vec{∇}·\vec{Π} is a scalar potential, \vec{N}_{Π} and \vec{N}_{φ} are the unit normal vectors.

The first case. The localization region of self-oscillations is the volume V_{Π} limited by the simply connected closed surface S_{Π}:

$$\vec{Π} = Π {\vec{i}}_{Π} , \qquad d {\vec{S}}_{Π} = {\vec{N}}_{Π} {dS}_{Π} , \qquad {\vec{N}}_{Π} = \frac{\vec{∇} Π}{\vert \vec{∇} Π \vert}$$

The Laplacian of a vector function of the polarization potential \vec{Π}=Π\vec{i}_{Π} is:

$${\vec{∇}}^{2} \vec{Π} = {\vec{i}}_{Π} ( {\vec{∇}}^{2} Π ) + 2 ( \vec{∇} Π \cdot \vec{∇} ) {\vec{i}}_{Π} + Π ( {\vec{∇}}^{2} {\vec{i}}_{Π} )$$

The divergence of the Laplacian of a vector function of the polarization potential \vec{Π}=Π\vec{i}_{Π} is:

$$\vec{∇} \cdot ( {\vec{∇}}^{2} \vec{\prod } ) = ( {\vec{∇}}^{2} \prod ) ( \vec{∇} \cdot {\vec{i}}_{\prod } ) + \frac{d}{d {i}_{\prod }} ( {\vec{∇}}^{2} \prod ) + 2 \vec{∇} \cdot ( ( \vec{∇} \prod \cdot \vec{∇} ) {\vec{i}}_{\prod } ) + \prod ( \vec{∇} \cdot ( {\vec{∇}}^{2} {i}_{\prod } ) ) + ( \vec{∇} \prod ) \cdot ( {\vec{∇}}^{2} {\vec{i}}_{\prod } ) \qquad 5 - 1$$

The gradient of the polarization potential modulus Π=|\vec{Π}| is expressed through the derivative with respect to the direction of the unit normal vector \vec{N}_{Π} (normal derivative):

$$\vec{∇} \prod = {\vec{N}}_{\prod } \frac{d \prod }{d {N}_{\prod }}$$

The third term in the expression (5-1) takes the following form:

$$2 \vec{∇} \cdot ( ( \vec{∇} \prod \cdot \vec{∇} ) {\vec{i}}_{\prod } ) = 2 \vec{∇} \cdot ( \frac{d \prod }{d {N}_{\prod }} \frac{d {\vec{i}}_{\prod }}{d {N}_{\prod }} ) = 2 \frac{d \prod }{d {N}_{\prod }} ( \vec{∇} \cdot ( \frac{d {\vec{i}}_{\prod }}{d {N}_{\prod }} ) ) + 2 ( \vec{∇} ( \frac{d}{d {N}_{\prod }} ) ) \cdot ( \frac{d {\vec{i}}_{\prod }}{d {N}_{\prod }} ) \qquad 5 - 2$$

The divergence of a vector function of the polarization potential \vec{Π}=Π\vec{i}_{Π} is:

$$\vec{∇} \cdot \vec{\prod } = \prod ( \vec{∇} \cdot {\vec{i}}_{\prod } ) + \frac{d \prod }{d {i}_{\prod }}$$

The Laplacian of the divergence of a vector function of the polarization potential \vec{Π}=Π\vec{i}_{Π} is:

$${\vec{∇}}^{2} ( \vec{∇} \cdot \vec{\prod } ) = {\vec{∇}}^{2} ( \prod ( \vec{∇} \cdot {\vec{i}}_{\prod } ) ) + {\vec{∇}}^{2} ( \frac{d \prod }{d {i}_{\prod }} ) \qquad 5 - 3$$

The first term in the expression (5-3) is the Laplacian of the product of two scalar functions—polarization potential modulus Π=|\vec{Π}| and the divergence of its unit vector \vec{∇}·\vec{i}_{Π}.

The Laplacian of the product of two scalar functions is defined as follows:

$${\vec{∇}}^{2} ( Φ Ψ ) = Ψ ( {\vec{∇}}^{2} Φ ) + 2 ( \vec{∇} Φ ) \cdot ( \vec{∇} Ψ ) + Φ ( {\vec{∇}}^{2} Ψ )$$

H M Thomas et al., New Journal of Physics, Volume 10, March 2008

Therefore, the first term in the expression (5-3) can be written as:

$${\vec{∇}}^{2} ( \prod ( \vec{∇} \cdot {\vec{i}}_{\prod } ) ) = ( {\vec{∇}}^{2} \prod ) ( \vec{∇} \cdot {\vec{i}}_{\prod } ) + 2 ( \vec{∇} \prod ) \cdot ( \vec{∇} ( \vec{∇} \cdot {\vec{i}}_{\prod } ) ) + \prod ( {\vec{∇}}^{2} ( \vec{∇} \cdot {\vec{i}}_{\prod } ) ) \qquad 5 - 4$$

As before, the gradient of the polarization potential modulus Π=|\vec{Π}| through the derivative in the direction of the unit normal vector \vec{N}_{Π} will be written. Thus, the following expression for the second term in (5-4) results:

$$2 ( \vec{∇} \prod ) \cdot ( \vec{∇} ( \vec{∇} \cdot {\vec{i}}_{\prod } ) ) = 2 \frac{d \prod }{d {N}_{\prod }} \frac{d}{d {N}_{\prod }} ( \vec{∇} \cdot {\vec{i}}_{\prod } ) \qquad 5 - 5$$

Applying (5-5) to (5-4) and then to (5-3), the following expression for the Laplacian of the divergence of a vector function of the polarization potential \vec{Π}=Π\vec{i}_{Π} is arrived:

$${\vec{∇}}^{2} ( \vec{∇} \cdot \vec{\prod } ) = ( {\vec{∇}}^{2} \prod ) ( \vec{∇} \cdot {\vec{i}}_{\prod } ) + 2 \frac{d \prod }{d {N}_{\prod }} \frac{d}{d {N}_{\prod }} ( \vec{∇} \cdot {\vec{i}}_{\prod } ) + \prod ( {\vec{∇}}^{2} ( \vec{∇} \cdot {\vec{i}}_{\prod } ) ) + {\vec{∇}}^{2} ( \frac{d \prod }{d {i}_{\prod }} ) \qquad 5 - 6$$

Compare the obtained expression with the previously found expression of the divergence of the Laplacian of a vector function of the polarization potential \vec{Π}=Π\vec{i}_{Π}:

$$\vec{∇} \cdot ( {\vec{∇}}^{2} \vec{\prod } ) = ( {\vec{∇}}^{2} \prod ) ( \vec{∇} \cdot {\vec{i}}_{\prod } ) + \frac{d}{d {i}_{\prod }} ( {\vec{∇}}^{2} \prod ) + 2 \vec{∇} \cdot ( ( \vec{∇} \prod \cdot \vec{∇} ) {\vec{i}}_{\prod } ) + \prod ( \vec{∇} \cdot ( {\vec{∇}}^{2} {\vec{i}}_{\prod } ) ) + ( \vec{∇} \prod ) \cdot ( {\vec{∇}}^{2} {\vec{i}}_{\prod } )$$

$$Where :$$

$$2 \vec{∇} \cdot ( ( \vec{∇} \prod \cdot \vec{∇} ) {\vec{i}}_{\prod } ) = 2 \vec{∇} \cdot ( \frac{d \prod }{d {N}_{\prod }} \frac{d {\vec{i}}_{\prod }}{d {N}_{\prod }} ) = 2 \frac{d \prod }{d {N}_{\prod }} ( \vec{∇} \cdot ( \frac{d {\vec{i}}_{\prod }}{d {N}_{\prod }} ) ) + 2 ( \vec{∇} ( \frac{d \prod }{d {N}_{\prod }} ) ) \cdot ( \frac{d {\vec{i}}_{\prod }}{d {N}_{\prod }} )$$

The Laplace operator always commutes with the divergence operator:

$$\{ {\vec{∇}}^{2} ( \vec{∇} \cdot \vec{\prod } ) \vec{∇} \cdot ( {\vec{∇}}^{2} \vec{\prod } ) \} ≡ 0 \qquad 5 - 7$$

Comparing (5-6) and (5-1) and using the expression (5-7), the following relations are arrived:

### Relation:

$${\{ ( {\vec{∇}}^{2} Π ) ( \vec{∇} \cdot {\vec{i}}_{Π} \to ) - ( {\vec{∇}}^{2} Π ) ( \vec{∇} \cdot {\vec{i}}_{Π} ) \}}_{Relation :} ≡ 0$$

$${Π \{ {\vec{∇}}^{2} ( \vec{∇} \cdot {\vec{i}}_{Π} ) - ( \vec{∇} \cdot ( {\vec{∇}}^{2} {\vec{i}}_{Π} ) ) \} ≡ 0}_{Relation :}$$

$${2 \frac{d \prod }{d {N}_{Π}} \{ \frac{d}{d {N}_{Π}} ( \vec{∇} \cdot {\vec{i}}_{Π} ) - \vec{∇} \cdot \frac{{\vec{di}}_{Π}}{d {N}_{Π}} \} \neq 0}_{Relation :} \qquad A$$

$${\{ {\vec{∇}}^{2} ( \frac{d Π}{d {i}_{Π}} ) - \frac{d}{d {i}_{Π}} ( {\vec{∇}}^{2} Π ) \} \neq 0}_{Relation :} \qquad B$$

$${( A ) + ( B ) = 2 ( \vec{∇} ( \frac{d \prod }{d {N}_{Π}} ) ) \cdot ( \frac{{\vec{di}}_{Π}}{d {N}_{Π}} ) + ( \vec{∇} \prod ) \cdot ( {\vec{∇}}^{2} {\vec{i}}_{Π} )}_{Relation :} \qquad C$$

Relation (A) contains a commutator of the normal derivative and the operator of the divergence:

$$2 \frac{d Π}{d {N}_{Π}} \{ \frac{d}{d {N}_{Π}} ( \vec{∇} \cdot {\vec{i}}_{Π} ) - \vec{∇} \cdot \frac{{\vec{di}}_{Π}}{d {N}_{Π}} \} \neq 0$$

For the vector function \vec{Π}=Π\vec{i}_{Π}, a commutator of the normal derivative and the operator of the divergence is as follows:

$$\frac{d}{d {N}_{Π}} ( \vec{∇} \cdot \vec{Π} ) - \vec{∇} \cdot \frac{d \vec{Π}}{d {N}_{Π}} = Π \{ \frac{d}{d {N}_{Π}} ( \vec{∇} \cdot {\vec{i}}_{Π} ) - \vec{∇} \cdot \frac{{\vec{di}}_{Π}}{d {N}_{Π}} \}$$

Therefore, the relation (A) can be written as:

$$\frac{2}{Π} \frac{d Π}{d {N}_{Π}} \{ \frac{d}{d {N}_{Π}} ( \vec{∇} \cdot \vec{Π} ) - \vec{∇} \cdot \frac{d \vec{Π}}{d {N}_{Π}} \} \neq 0 \qquad ( A 1 )$$

The external electrostatic field is assumed to be defined by the potential φₒᵤₜ=cₒᵤₜ(\vec{∇}·\vec{Π}).

In the first approximation, the effect of the external electrostatic field on commutativity of the normal derivative and operator of the divergence can be described by the first-order linear equation:

$${c}_{0} \frac{d}{d {N}_{Π}} ( \vec{∇} \cdot \vec{Π} ) + {c}_{out} ( \vec{∇} \cdot \vec{Π} ) = {f}_{1} \qquad ( A 2 )$$

Here

$${c}_{0} = \frac{2}{Π} \frac{d Π}{d {N}_{Π}}$$

is the amplitude of the external scalar potential, f₁ is all the remaining members of the expressions (A1), (B) and (C).

Thus, if self-oscillations of the polarization potential \vec{Π}=Π\vec{i}_{Π} are localized in a volume V_{Π} limited by a simply connected closed surface S_{Π}, the reaction of the self-oscillating system to an external electrostatic field is described by a first-order linear differential equation.

The second case. The localization region of self-oscillations is the volume V_{φ} limited by the simply connected closed surface S_{φ}:

$$φ = - \vec{∇} \cdot \vec{Π} , d {\vec{S}}_{φ} = {\vec{N}}_{φ} d {S}_{φ} , {\vec{N}}_{φ} = \frac{\vec{∇} φ}{\vert \vec{∇} φ \vert}$$

Assume that the external electrostatic field is defined by the potential φₒᵤₜ=cₒᵤₜ(\vec{∇}·\vec{Π}).

In the first approximation, the effect of the external electrostatic field is described by the following equation:

$${\vec{∇}}^{2} φ + {φ}_{o u t} = {f}_{2} , here {f}_{2} = \vec{∇} \cdot ( {\vec{∇}}^{2} Π )$$

Using the relation between the Laplacian of a scalar function and its normal derivative:

$${\vec{∇}}^{2} φ = \frac{{d}^{2} φ}{d {N}_{φ}^{2}} + \frac{d φ}{d {N}_{φ}} ( \vec{∇} \cdot {\vec{N}}_{φ} )$$

If self-oscillations of the polarization potential \vec{Π}=Π\vec{i}_{Π} are localized in a volume V_{φ} limited by a simply connected closed surface S_{φ}, the reaction of the self-oscillating system to an external electrostatic field is described by the second-order linear differential equation:

$$\frac{{d}^{2} φ}{d {N}_{φ}^{2}} + \frac{d φ}{d {N}_{φ}} ( \vec{∇} \cdot {\vec{N}}_{φ} ) + {c}_{o u t} φ = {f}_{2} , where {f}_{2} = \vec{∇} \cdot ( {\vec{∇}}^{2} Π )$$

### Result:

1. If the electric charge density occupies a volume V_{Π} limited by a simply connected closed surface S_{Π}, the reaction of the self-oscillating system to an external electrostatic field is described by the first-order differential equation:

$$\vec{\prod } = \prod {\vec{i}}_{\prod } ,$$

$$d {\vec{S}}_{\prod } = {\vec{N}}_{\prod } {dS}_{\prod } ,$$

$${\vec{N}}_{\prod } = \frac{\vec{∇} \prod }{\vert \vec{∇} \prod \vert}$$

$${c}_{0} \frac{d}{{dN}_{\prod }} ( \vec{∇} \cdot \vec{\prod } ) + {c}_{out} ( \vec{∇} \cdot \vec{\prod } ) = {f}_{1}$$

2. If the electric charge density occupies a volume V_{φ} limited by a simply connected closed surface S_{φ}, the reaction of the self-oscillating system to an external electrostatic field is described by the second-order differential equation:

$$φ = - \vec{∇} \cdot \vec{\prod } ,$$

$$d {\vec{S}}_{φ} = {\vec{N}}_{φ} {dS}_{φ} ,$$

$${\vec{N}}_{φ} = \frac{\vec{∇} φ}{\vert \vec{∇} φ \vert}$$

$$\frac{{d}^{2} φ}{{dN}_{φ}^{2}} + \frac{d φ}{{dN}_{φ}} ( \vec{∇} \cdot {\vec{N}}_{φ} ) + {c}_{out} φ = {f}_{2}$$

According to H M Thomas et al., New Journal of Physics, Volume 10, March 2008, a solution of the nonhomogeneous differential equation can be expressed as the sum of a general solution of the homogeneous equation and a partial solution of the nonhomogeneous one.

The homogeneous equations of first and second order have the following solutions:

$${a}_{0} \frac{dy}{dt} + {a}_{1} y = 0$$

$$y = {Ce}^{- \frac{{a}_{1}}{{a}_{0}} t}$$

Mathematical Handbook for scientists and engineers. Definitions, Theorems and formulas for reference and review. G. A. Korn, T. M. Korn, McGraw-Hill Book Company, New York, San Francisco, Toronto, London, Sydney, 1968.

$${a}_{0} \frac{{d}^{2} y}{{dt}^{2}} + {a}_{1} \frac{dy}{dt} + {a}_{2} y = 0$$

$$y = {C}_{1} {e}^{{s}_{1} t} + {C}_{2} {e}^{{s}_{2} t}$$

$${s}_{1 , 2} = \frac{- {a}_{1} \pm \sqrt{{a}_{1}^{2} - 4 {a}_{0} {a}_{2}}}{2 {a}_{0}}$$

Here is the case of a homogeneous component. In this approximation, the relevant solutions can be written as exponentially decreasing functions:

$${c}_{0} \frac{d}{{dN}_{\prod }} ( \vec{∇} \cdot \vec{\prod } ) + {c}_{out} ( \vec{∇} \cdot \vec{\prod } ) = {f}_{1}$$

$$\vec{∇} \cdot \vec{\prod } ∼ {Ce}^{- \frac{{c}_{out 1}}{{c}_{0}} {ξ}_{\prod }}$$

$$\frac{{d}^{2} φ}{{dN}_{φ}^{2}} + \frac{d φ}{{dN}_{φ}} ( \vec{∇} \cdot {\vec{N}}_{φ} ) + {c}_{out} φ = {f}_{2}$$

$$φ ∼ {C}_{1} {e}^{{s}_{1} {ξ}_{φ}} + {C}_{2} {e}^{{s}_{2} {ξ}_{φ}}$$

$${s}_{1 , 2} = - δ \pm \sqrt{{δ}^{2} - {c}_{out}}$$

Here
ξ_{Π} is an amount of displacement of point (x,y,z) in the normal direction \vec{N}_{Π},
ξ_{φ} is an amount of displacement of point (x,y,z) in the normal direction \vec{N}_{φ},

$$δ = \frac{1}{2} ( \vec{∇} \cdot {\vec{N}}_{φ} )$$

is the damping coefficient, C, C₁ and C₂ are some constants,
cₒᵤₜ is an amplitude of the external scalar potential.

An exponent α of the function of type e^{-ar} is called a damping constant. According to quantum mechanics, the damping constant for intra-atomic processes is a linear function of the nucleus charge Z. L. A. Vainstein. Electromagnetic waves.-Second ed., Moscow, Radio I Svyaz, 1988,-440p. ISBN 5-256-00064-0. The dependence of the wave function of electrons in an atom Ψₙ(r,t) as a function of time can be written as:

$${Ψ}_{n} ( r , t ) = {ψ}_{n} ( r ) {e}^{i \frac{{E}_{n}}{ℏ} t}$$

The function ψₙ(r) describes dependence of the wave function of electrons in the atom as a function of spatial coordinates and can be expressed as:

$${ψ}_{n} ( r ) = {Nr}^{( {n}^{⋆} - 1 )} {e}^{- α r} Y ( θ , ϕ )$$

Stuart Kauffman. Investigations. Oxford University Press, 19 Sep. 2002, 308p.

Where N is a normalizing multiplier, Y(θ,ϕ), the normalizing multiplier being functions of spherical harmonics, n is the effective quantum number, and α is the real attenuation constant. The real attenuation constant α is given by:

$$α = \frac{Z - s}{{n}^{⋆} {a}_{0}}$$

Here s is the screening constant, Z is the nucleus charge, and α₀ is the Bohr radius.

In case of hydrogen-like atom α:

$$α = \frac{Z}{{na}_{0}}$$

Here Z is the nucleus charge and n is the principal quantum number.

Comparing the exponents, two dependences of the amplitude of the external scalar potential cₒᵤₜ on the nucleus charge Z result.
1. If an electric charge is distributed by volume V_{Π} which is limited by a simply connected closed surface S_{Π}, an amplitude of the external scalar potential cₒᵤₜ is a linear function of the nucleus charge Z.
2. If an electric charge is distributed by volume V_{φ} which is limited by a simply connected closed surface S_{φ}, an amplitude of the external scalar potential cₒᵤₜ is a quadratic function of the nucleus charge Z.

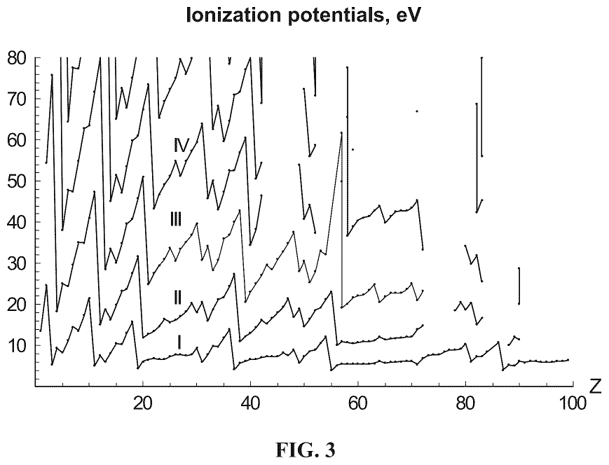

The energies of internal electrons in many electron atoms are usually obtained from experimental X-ray spectra. However, in this way it is possible to measure only the energies of the orbitals occupied in the atom; therefore, the curves lack the most interesting initial sections with small Z. Starting from the principal quantum number equal to three, the form of the graphs is very far from parabolic ones. The numerical values of the energy of electrons on individual orbitals differ significantly from the corresponding values obtained from the analysis of ionization potentials. As will be clear from the data below, this is because X-ray spectrums make it possible to determine the energy of electrons in an excited degenerate state. It would be much more logical to determine the energies of electron states by analyzing ionization potentials, data on which have been accumulated during numerous experiments over the past 100 years. However, until now, no one has done this due to rather subjective reason. The fact is that historically, and this can be seen from any textbook on general chemistry, the graphs of the first, second, third, and so on, ionization potentials versus the nuclear charge are usually represented in the form shown in FIG. 3.

All that can be seen is that the ionization potentials decrease from the top to bottom in the groups and increase from left to right along the periods in periodic table of chemical elements. In this representation, the ionization energies of electrons of different atoms with different electron configuration can be observed. At the same time, knowledge of the energy of electron states in complex atoms is fundamentally important both for understanding the mechanism of the formation of chemical bonds and for the synthesis of new materials with specified properties, both inorganic and organic. It is very difficult if not impossible to obtain these data by theoretical calculations for atoms with more than three electrons. To solve this problem, proceed as follows.

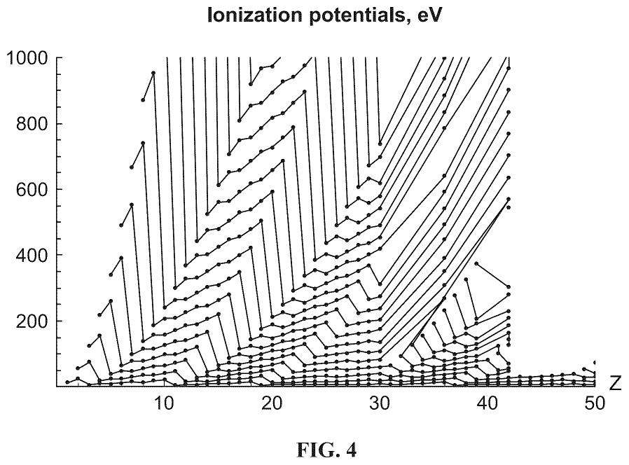

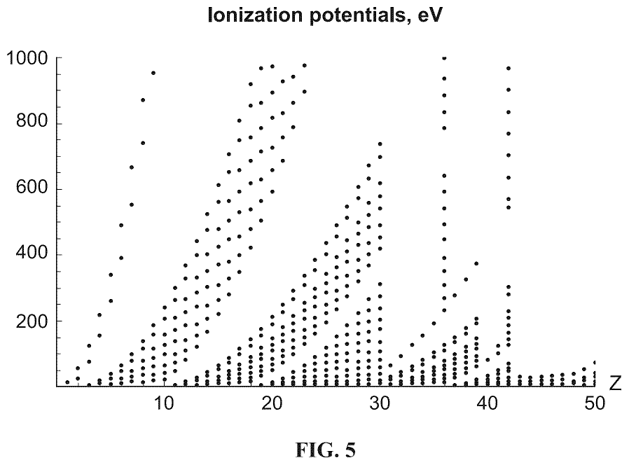

The experimental value of the ionization potentials by bold dots in the figure with reduced scale of the ionization potentials curves constructed according to generally accepted principle as in FIG. 3. See also FIG. 4 and FIG. 5.

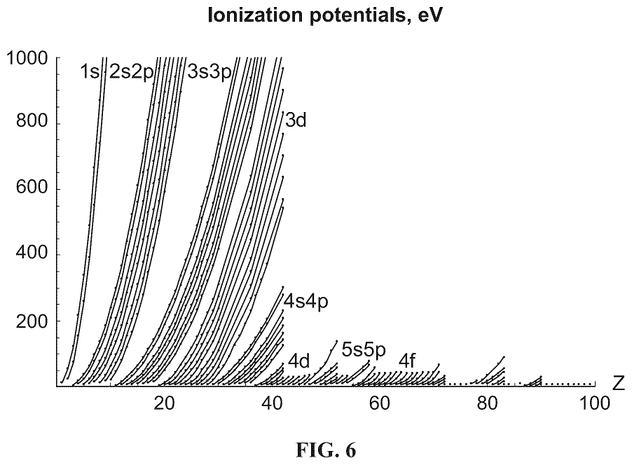

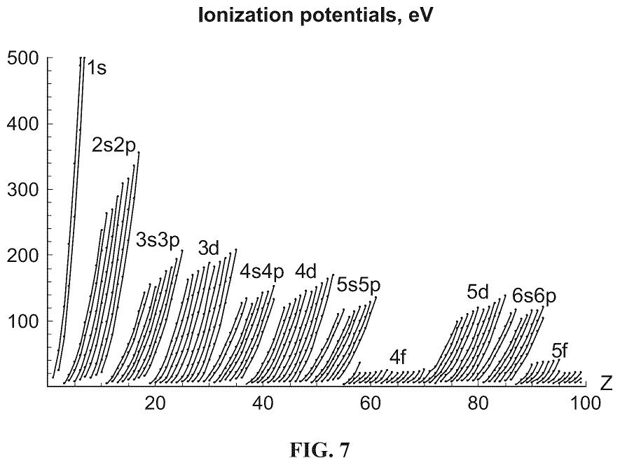

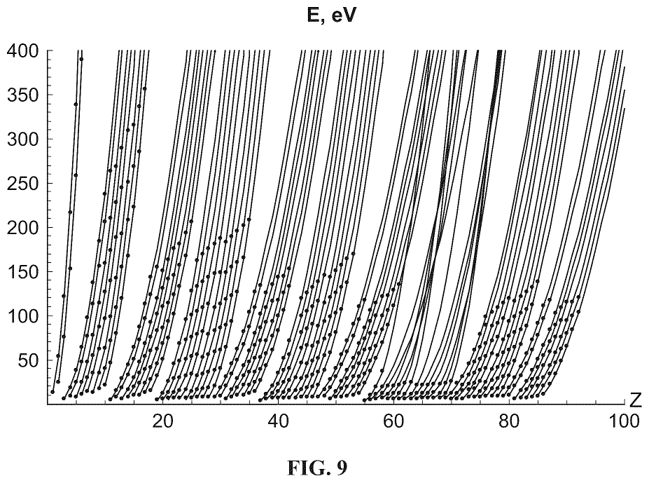

Next, remove the connecting lines and leave only experimental dots. After such a simple transformation, even a five-year-old would start connecting dots differently. The resulting graph demonstrates the amazing power of the expression “ . . . connecting dots” (FIG. 6, FIG. 7 and FIG. 9).

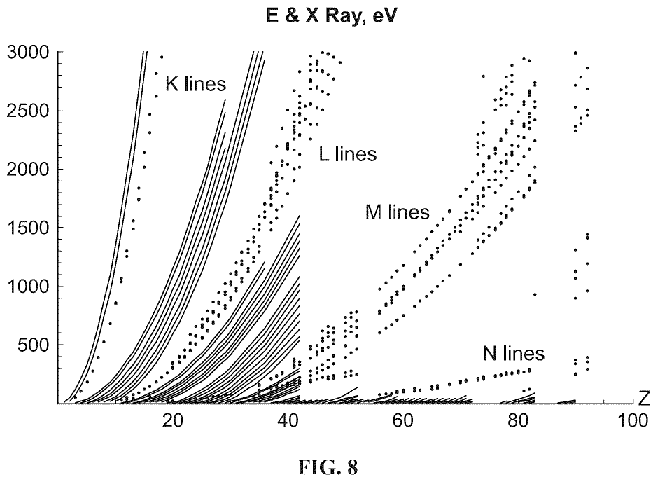

From the data in FIG. 8 it can be seen that X-ray radiation energies relate to degenerate electron states and cannot provide enough information to identify correct electron configuration of each electron and the energy level in the atom. At the same time, from the data shown in FIG. 6, FIG. 7 and FIG. 9, an unambiguous and unexpected conclusion can be made about electron configuration of the atoms in the rows of the periodic table starting with potassium, rubidium, and cesium. The first ionization potentials for elements with atomic numbers (Z) equal to 19 (K), 37 (Rb) and 55 (Cs) clearly belong to parabolas corresponding to the states 3d1 for potassium, 4d1 for rubidium and 4f1 for cesium and not to s-1 states as currently assumed in the physics and chemistry textbooks. It means that Aufbau principle and Madelung's rule used to define electron configuration of an atomic species should be corrected. Does it matter? In the end, chemists worked successfully before the creation of quantum mechanics. As for quantum chemistry, it still has not lived up to the hopes placed on it. Materials with the given properties still cannot be obtained. What is the reason for this situation? The fact is that modern chemistry still relies on the principles of thermodynamics based on considering the parameters describing the behavior of astronomical number of particles. However, thermodynamics does nothing when it is required to create a chemical bond between two or several atoms. In such cases, knowledge of the electron configuration of atoms becomes fundamentally important and a necessary condition. However, even though modern technology allows individual atoms to be placed very close to each other, it is not possible to force them to form a chemical bond. The analysis of chemical bond formation is usually focused on the formation of energetically favorable molecular orbitals. The fact that the strengths of the electric and magnetic fields are vector functions is not considered. After all, energy is a scalar.

Shown above is that the electric charge density in an atom can be described as a localized and electrically neutral ρₑ=ρₙ self-oscillating system of the polarization potential \vec{Π}.

Consider the polarization potential from the position of energy.

A vector function called Hertz polarization potential is introduced. In the international system of units SI, the polarization potential is defined as follow:

$$\vec{\prod }$$

$$A \text{?} = \frac{1}{{υ}^{2}} \frac{\partial \vec{\prod }}{\partial t} , φ = - ∇ \text{?} \cdot \prod \text{?}$$

$$E \text{?} = - \vec{∇} φ - \frac{\partial \vec{A}}{\partial t} , B \text{?} = ∇ \text{?} \times A \text{?}$$

$$∇ \text{?} \cdot \vec{E} = \frac{ρ}{{εε}_{0}} , ∇ \text{?} \times B \text{?} = \frac{1}{{υ}^{2}} \frac{\partial E \text{?}}{\partial t} + {μμ}_{0} j \text{?}$$

$$\text{?} \text{indicates text missing or illegible when filed}$$

James Clerk Maxwell, A treatise on electricity and magnetism. Moscow, Science, 1989, ISBN 5-02-000042-6. L. A. Vainstein. Electromagnetic waves.-Second ed., Moscow, Radio I Svyaz, 1988,-440p. ISBN 5-256-00064-0.

Assume that the polarization potential function describes a monochromatic wave. Also assume that the independent variable in of this function is a linear combination of coordinates and time ωt+kξ.

$$\vec{\prod } = \vec{F} ( ω t + k ξ )$$

Here ξ—the path travelled by the wave front, ω—oscillation frequency, k—wave number.

With this choice, the directional derivative with respect to the propagation of the wave front is proportional to the time derivative:

$$\frac{\partial }{\partial ξ} = \frac{1}{υ} \frac{\partial }{\partial t}$$

Here ν=ω/k—phase velocity.

As a result the first directional derivative of the polarization potential function defines the vector potential. The second directional derivative of the polarization potential function defines the electric field strength. The third directional derivative defines electric current density.

\vec{Π}˜\vec{A},\vec{Π}″˜\vec{E},\vec{Π}′″˜\vec{j}

Accordingly, expression

$$\overline{Φ} \to \frac{\partial \overline{Φ}}{\partial t} \to emf \to \overline{j} \to \overline{A} \to \overline{E} , \overline{B}$$

will take a simpler form:

\vec{Π}→\vec{Π}′→\vec{Π}″→\vec{Π}′″→\vec{Π}′→\vec{Π}″→\vec{Π}′″→ . . .

By definition, the energy of the electromagnetic field is defined as follows:

$$W = {\int \int \int }_{V} ( \frac{\vec{E} \vec{D}}{2} + \frac{\vec{B} \vec{H}}{2} ) dV$$

L. A. Vainstein. Electromagnetic waves.-Second ed., Moscow, Radio I Svyaz, 1988,-440p. ISBN 5-256-00064-0.

The corresponding equation of dimensionalities has the form:

Joule=[\vec{E}]² meter³

The dimensionality of the electric field strength can be expressed through the vector potential:

$$[ \vec{E} ] = \frac{[ \vec{A} ]}{second}$$

The dimensionality of the vector potential through the polarization potential is expressed as:

$$[ \vec{A} ] = \frac{{second}^{2}}{{meter}^{2}} \frac{[ \vec{Π} ]}{second} = \frac{second [ \vec{Π} ]}{{meter}^{2}}$$

Therefore, the dimensionality of the electric field strength can be expressed as follows:

$$[ \vec{E} ] = \frac{[ \vec{Π} ]}{{meter}^{2}}$$

Accordingly, the expression for the energy takes the form:

$$Joule = \frac{{[ \vec{Π} ]}^{2}}{meter}$$

Therefore, the energy of the electromagnetic field W is proportional to the directional derivative in of the square of the module of the polarization potential.

$$W \sim \frac{d {\vert \vec{Π} \vert}^{2}}{d ξ}$$

Here ξ—the path travelled by the wave front.

The last expression means that it is fundamentally important to unambiguously define the directions of propagation of the first, second and third derivatives of the polarization potential when solving problems related to the possible loss of electromagnetic energy.

\vec{Π}′˜\vec{A},\vec{Π}″˜\vec{E},Π′″˜\vec{j}

At the differential level, the direction is always preserved. Therefore, the unambiguity of defining the direction must be considered from the point of view of the chemical bond formation. If the direction is maintained and vector potential and vectors of the strength of electric and magnetic fields are well-defined, then the losses of electromagnetic energy during chemical bonds formation can be avoided. If the direction is averaged, then there is a loss of energy. The more averaging, the greater the loss of energy and less probability for the creation of chemical bonds and a molecule as the self sustained oscillating system in the result of interaction of two or more atoms. According to Mathematical Handbook for scientists and engineers, Definitions, Theorems and formulas for reference and review. G. A. Korn, T. M. Korn, McGraw-Hill Book Company, New York, San Francisco, Toronto, London, Sydney, 1968; Mathematical Encyclopedic Dictionary, Chief Ed. A. M. Prokhorov.-Moscow, Soviet Encyclopedia, 1988,-847p.; And L. A. Vainstein. Electromagnetic waves.-Second ed., Moscow, Radio I Svyaz, 1988,-440p. ISBN 5-256-00064-0, the normal of the level surface is well-defined at the interface/border between two media which have different values of dielectric and magnetic permeability. Therefore, the best way to exclude the losses of energy and direction of the vectors of the strength of electric and magnetic fields in the process of autocatalytic reactions, is to carry out reactions on the surfaces of multilayer structures composed of thin/monoatomic layers having different values of the dielectric constant and magnetic susceptibility. For technical/engineering purposes, for example in case of high-temperature superconductors or reactors for chemical reactions, such multilayer structures must be specially designed. As for the live cells of the human body, such engineering solutions have already been implemented as a result of evolution. Good examples are structures of axones with their myelin sheath integrity of which is crucial for nerve signals transmission or endoplasmic reticulum being critical for proteins and lipids synthesis as well as for the proteins folding.

Emphasis is placed once again that the unambiguous assignment of vector functions associated with electromagnetic interaction and minimization of energy losses are the main principles of insuring the stability of complex hierarchical self-sustained systems.

In addition, as follows from FIG. 2, it is very important to maintain a balance between the two types of negative feedback, potential and solenoidal. Taking into account that

$$B = μϵ \frac{\partial }{\partial t} ( ∇ \times )$$

and Faraday's law:

$$\vec{∇} \times \vec{E} = - \frac{\partial \vec{B}}{\partial t} .$$

Assume that in the process of the molecules, the potential component will define chemical bonds formed due to Coulombic interaction along the strength lines, while the solenoidal part is associated with the formation of helices, which is characteristic of such multi-level molecules as proteins and DNA. According to the schemes presented in FIG. 1 and FIG. 2, the stability of the newly formed molecule will also depend on the balance of the above two types of negative feedback. It is important to take into account that the energy involved in the formation of helices of DNA and proteins is very small, on the order of 0.01 Ev, Kenso Soai. Amplification of chirality. Springer Berlin Heidelberg, 2010, 205 p., and therefore it is very sensitive to the slightest change in values of the first and second derivatives of polarization potential in respect to both time and direction. In general, the constituent parts of DNA, RNA (chiral bases, chiral phosphate ester units, D- and L-sugar components) and proteins (amino acids) tend to form racemic mixtures. Distortion or rupture of potential or solenoidal negative feedback leads to an irreversible change in the chirality of their components and, accordingly, to DNA/RNA mutations and disruption of proteins conformation. The imbalance between the two types of negative feedback resulting in mutations can be caused by environmental risk factors such as sunlight, radiation or smoking.

Mutations in biomolecules can also appear as a result of the action of specific chemicals and/or chemical compounds containing free radicals. But even in the absence of the aforementioned risk factors, some mutations are “naturally occurring” in the process of cells division and DNA replication. In addition, DNA mutations also lead to the inability of the cell's machinery/proteins responsible for DNA repair to work efficiently. This inevitably leads to the accumulation of both DNA mutations and mutant proteins in the live cells over time. That is why age is considered as the biggest risk factor that significantly increases the likelihood of degenerative diseases.

From the point of view of a stable system, proteins play role of receiving-transmitting antennas (receptors and ligands) and signals (polypeptides) in a live cells. Naturally, the incorrect conformation of proteins inevitably leads to a violation of negative feedback and consequently, the stability of the body's cells. Currently, life is defined as the existence of proteins involved in autocatalytic reactions. Stuart Kauffman. Investigations. Oxford University Press, 19 Sep. 2002, 308p. Autocatalytic reactions in live cells are non-linear in nature. Stuart Kauffman. Investigations. Oxford University Press, 19 Sep. 2002, 308p.

It is at the moment of the manifestation of the nonlinearity of the output products of auto catalytic reactions that the concentration of D-amino acids, proteins containing D-amino acids and, therefore, defective proteins with wrong conformation increases sharply. As a result of this process, the destruction of negative feedbacks regulating the stability of a live cell takes on an avalanche-like character. At such a moment, previously healthy people develop symptoms of the disease, or chronically sick patients develop an acute phase of the disease. The type of disease depends on which of the 200 different cells of the body will create optimal conditions for the onset non-linear autocatalytic reactions. It is well known that there are many mechanisms in live cells to correct DNA defects and disassemble dysfunctional proteins. In this regard, it is appropriate to mention the role of p53 protein, which is called “the guardian angel of the genome.” Guillermina Losano, Arnold J. Levine, Ed. The p53 protein form cell regulation to cancer. Cold Spring Harbor Perspectives in Medicine. Despite this, no one has yet been able to avoid the aging process and degenerative diseases. This means that there are hitherto unknown pathogenic factors leading to a distortion of the helices of DNA and proteins and overwhelming defense mechanisms of live cells. This obstacle prevents successful treatment of degenerative diseases. The most advanced methods of modern medicine are based on establishing a correlation between certain DNA mutations and one or another degenerative disease. After establishing the correlation, mainly two options are used. The first is to inhibit the overexpressed proteins corresponding to the established DNA mutations and obviously critical for the progress of the disease. The second option is associated with the recently appeared ability to synthesize “correct” or absent proteins by manipulations with mRNA. Both methods lead to the changes not only in pathology affected cells but also in healthy ones and therefore, may cause harmful side effects. In other words, both methods unambiguously lead to the further destruction of negative feedback and, consequently, to undermining the sanity of a living system. In order to ensure this stability and circumvent the limitations of existing therapies it is necessary to find out the nature of the very first changes leading to the onset of diseases-distortion of DNA and proteins helices. This means that one needs to pay attention to the balance of potential and solenoidal components in the scheme of the atom as an auto-oscillation system of the polarization potential since it is on them that the perfection of chemical bonds and the formation of helices depends on (FIG. 2).

Despite tremendous progress in the development of life sciences over the past 70 years and huge number of talented scientists, modern medicine is still unable to do anything more than transform some acute diseases into chronic ones. And even such a result is very difficult to achieve and, in fact, it is a dream scenario and goal of modern therapy for degenerative diseases. The lack of the desired end result over such a long period of time is most likely due to the fact that some basic scientific concepts or models that are well established are actually incorrect.

Consider the logic of a new approach to the treatment of degenerative diseases and increasing the stability of the human body based models and data presented above.
1. The very possibility of the existence of any stable system/object from elementary particles to the human body exists solely due to the presence of the physical meaning of an arbitrary vector function.
2. The physical meaning of any vector function is that, within its domain of definition, a vector function generates/describes its own oscillating system, and in this sense, it is its source (integral component). At the same time, a vector function itself can be a differential component of a self-oscillating system of a higher level.
3. Vector functions form interconnected self-oscillating systems both at the differential level and at the integral level. Mathematically, there are no fundamental limitations on the lower or upper “limit”. In this sense, there is a hierarchy of differential and integral characteristics of the vector functions. Since the relationship between differential and integral components of the self-oscillating systems is described by the same operations of vector algebra, groups (sublevels) of characteristics formed in the image and likeness of a superior integral group can be distinguished in this hierarchy.
4. In accordance with the definition of self-oscillations, the life and death of a self-oscillating system depends on the presence or absence of an integral component.

This point requires deep reflection since it has both technical/practical and philosophical significance. It can be expected that the new level of technology, including new therapeutical methods, will rely on this conclusion. What is the primary: a particle as a source of field or a field, a feature (complex operator) of which can be called a particle.

The practical significance of these theses lies in the fact that human body can be considered as a result of the simultaneous action of a certain number of vector fields with a pronounced differential-integral hierarchy. Ensuring the stability of the organism, starting from the second level (biomolecules) and above, can be provided due to homeostasis in a broad sense.

There are mechanisms in live cells to correct DNA mutations and remove the misfolded proteins. The balance/homeostasis of integrations between tissues and organs is regulated by the nervous system/brain. The most vulnerable link in the hierarchical structure is the lower link at the level of chemical elements, since the organism itself cannot change anything with regard to the chemical elements and, most importantly, their isotope composition.

Natural abundance of isotopes of the one and the same chemical element is absolutely independent and out of control from the senior components of the hierarchical system/cells, tissues, organs, brain homeostasis.

In accordance with quantum mechanics and quantum chemistry theory, no surprises should be expected on this level as long one “chemical element” is not substituted by another with a different electron configuration within a biomolecule. The theoretical considerations of an atom and its nucleus are based on several postulates, which address stationary state or a quantum state with all observables independent of time. The modern theory of atom and nucleus does not encompass the existence of an energy source with negative feedback or a hierarchy of junior and senior components.

The disclosed mathematical model of a stable system enables one to describe an atom as a self-sustained oscillating system of a vector function of polarization potential with no postulates. In this light, the atom may be viewed as at least a two-level stable system with different fields existing inside and outside of the nucleus. Both fields are derivatives of the vector function of polarization potential. For example, fields of the vector potential and scalar potential are thought to be interconnected. Such consideration allows one to predict particularly important consequences for the formation of complex hierarchical molecules. Outside of a complex stable system, atoms of identical chemical element may assume the form of various natural isotopes. To be considered the foundation for such a system, atoms must satisfy a specific condition: they must possess an identical number of electrons, protons, and neutrons. Only these kinds of atoms may become a part of a multi-level hierarchical system of biological molecules in life. Stable isotopes with an identical number of protons and electrons in most cases, represent the lightest ones, which constitute 98.2% of human body weight. Monoisotopic phosphorus and sodium add another 1.28. Stable isotopes of essential elements add remaining 0.6%. In this case the preference should be given to ones with a minimal excess of neutrons over protons, meaning lightest isotopes.

Experiments conducted with DNA-like dust structures in a cryogenic discharge at microgravity on the International Space Station demonstrated the degeneration of plasma crystals structures at temperatures higher than 4 Kelvin and non-zero gravitation. V N Tsytovich et al., New Journal of Physics 9 (2007) 263; H M Thomas et al., New Journal of Physics, Volume 10, March 2008. To define the operators of vector algebra (vector function and its derivatives), it is imperative to always control unit vector normals to the surface level. Normals in biomolecules are defined by the thermodynamic state of atoms. The higher the temperature, the stronger the chaotic oscillations of the atoms and the stronger the averaging of the normal. The surface level normal is well defined at the interface between two media, provided that these media have different dielectric and magnetic permeability values. This implies that in order to mitigate the effect of temperature, protein synthesis must occur on some kind of layered surface. That explains why within living cells, proteins are created by ribosomes not at a random location in cytoplasm, but specifically on the surface of the endoplasmic reticulum (ER). It would be important to learn more about the electrical and magnetic properties of the ER structure. Gravitation effect in living cells is reduced because the building blocks for the construction of proteins (amino acids and ions) are suspended in the cytoplasm of cells and therefore have virtually near zero weight, even though having lightest ions would always help.

To restore healthy negative feedback or homeostasis, one must ensure the stability of complex biomolecules. For this purpose, ambiguity/uncertainty or degeneration should be aimed to be excluded on the level of amino acids (right-handed chirality) as well as on the level of chemical elements (isotopes with identical number of protons and neutrons).

The problem is that the natural abundance of isotopes most often does not favor the appropriate ratio between protons and neutrons. The balance is quite good for the atoms of C, O, and N (Table 1), but unfortunately is not acceptable for most of the essential elements. Together with food, water and air, human beings get a mixture of stable natural isotopes, which become the building blocks for all biomolecules found in our body, including polypeptides and proteins. When isotope substitution takes place in “inorganic materials”, it may lead to minor changes in the physical and chemical properties. However, the substitution of light isotopes with heavy ones in biomolecules manifests the onset of pathological changes and often becomes a matter of life and death.

This process can happen in two different ways. The first one is related to the effect of “isotope-induced chirality” and subsequent amplification of the “wrong” chirality in the process of autocatalytic reactions. Stuart Kauffman. Investigations. Oxford University Press, 19 Sep. 2002, 308p. Autocatalytic reactions are non-linear and may cause a dramatic increase of the yield of the chiral product at certain points in time. The effect of isotope-induced changes of chirality has been characterized for H/De; 12C/13C; 14N/15N and 160/180. Kawasaki T. et al., Science 2009, 324, 492-495. Kawasaki T. et al., Chem. Int. Ed. 2011, 50, 8131-8133; [Angew. Chem. 2011, 123, 8281-8283. Arimasa Matsumoto et al., 2016 Dec. 5; 55 (49): 15246-15249. Avalos M. et al., Chem. Commun. 2000, 887-892. The same way isotope substitution in amino acids can lead to a change of their chirality and subsequently to a change of overall protein conformation and deregulated signaling within physiological systems that are responsible for maintaining homeostasis.

TABLE 1
Examples of isotopes and their abundance in nature, % (also referred herein
as “natural abundance” of isotopes, as distinct from enriched in specific isotope.

| C | N | H | | Mg | Fe | Zn | Se |
|---|---|---|---|---|---|---|---|
| C12-98.9 | N14-99.6 | H1-99.98 | O16-99.76 | Mg24-78.99 | Fe54-5.80 | Zn64-48.6 | Se74-0.90 |
| C13-1.10 | N15-0.4 | H2-0.02 | O17-0.04 | Mg25-10.00 | Fe56-91.72 | Zn66-27.90 | Se76-9.00 |
| | | | O18-0.20 | Mg26-11.01 | Fe57-2.20 | Zn67-4.10 | Se77-7.60 |
| | | | | | Fe58-0.28 | Zn68-18.80 | Se78-23.60 |
| | | | | | | Zn70-0.60 | Se80-9.70 |
| | | | | | | | Se82-9.20 |

Another mechanism of D-amino acids' formation in proteins and polypeptides is defining progression of degenerative pathologies in time. Original/proper conformation of a protein may be jeopardized due to several reasons such as isotope substitution, interaction with free radicals, radioactive radiation, and others. Once this occurs, the entire hierarchical structure of a biomolecule becomes damaged. L-amino acids gradually change their chirality toward normal racemic ratio, which inevitably results in progressive damage to the protein's topology and loss of function. This is precisely why degenerative diseases can never disappear once they are manifested within an organism. Far from being the primary reason, the presence of D-amino acids is usually just a symptom of misfolding, unfolding and aggregation of proteins as well as UPR-signatures of many degenerative pathologies, including Alzheimer's disease, Parkinson's Disease, Crohn's disease, and others.

Isotope substitution leading to reversal of biomolecules' “wrong” chirality has another side. Chirality amplification in autocatalytic reactions must be accompanied by accumulation of light isotopes in healthy cells and heavy isotopes in pathology-affected cells, tissues, and organs. This process obfuscates the ability to accurately picture the disease. A high concentration of heavy isotopes triggers local degradation of new biomolecules that were not involved in the development of the initial pathology. As a result, degenerative changes should not be analyzed as a singular disease, which will make both diagnostics and treatment much more difficult.

Mass spectrometry data obtained on various healthy and pathology-affected cells and tissues not only confirmed this finding, but also demonstrated that heavy isotope ratios of all essential elements are proportional to the degree of pathological changes.

For example, there is an expressive difference between light and heavy isotopes ratios of the same chemical elements found in healthy and leukemic lymphocytes. Lymphocytes are immune system cells. The data suggest that efficacy of T cells, B cells and natural killer cells is defined by isotope ratios of essential chemical elements, which is in complete agreement with the disclosed model of pathogenesis. U.S. Pat. No. 10,857,180; U.S. patent application Ser. Nos. 16/236,343; 16/973,169; PCT/US19/55770; PCT/US21/24433.

TABLE 2
Isotope ratios in Leukemia and Healthy
Lymphocytes. WO2022203684A1

| | | Natural | Healthy | Leukemia |
|---|---|---|---|---|
| Element | Isotope | Abundance % | Lymphocytes % | Lymphocytes % |
| Mg | Mg24 | 78.99 | 83.65 | 76.53 |
| | Mg25 | 10.00 | 9.57 | 7.57 |
| | Mg26 | 11.01 | 6.77 | 15.93 |
| Ca | Ca40 | 96.97 | 96.94 | 96.94 |
| | Ca42 | 0.64 | 1.11 | 0.10 |
| | Ca43 | 0.15 | 0.51 | 0.06 |
| | Ca44 | 2.06 | 1.14 | 2.04 |
| | Ca46 | 0.00004 | 0.11 | 0.26 |
| | Ca48 | 0.19 | 0.19 | 0.59 |
| Fe | Fe54 | 5.80 | 11.17 | 4.07 |
| | Fe56 | 91.72 | 87.55 | 91.40 |
| | Fe57 | 2.20 | 0.98 | 4.25 |
| | Fe58 | 0.28 | 0.28 | 0.28 |
| Zn | Zn64 | 48.60 | 58.83 | 45.59 |
| | Zn66 | 27.90 | 30.88 | 25.44 |
| | Zn67 | 4.10 | 3.74 | 3.85 |
| | Zn68 | 18.80 | 5.95 | 23.77 |
| | Zn70 | 0.60 | 0.60 | 1.42 |
| Rb | Rb85 | 72.16 | 92.12 | 68.19 |
| | Rb87 | 27.84 | 7.88 | 31.81 |
| Se | Se74 | 0.90 | 2.73 | 0.90 |
| | Se76 | 9.00 | 15.29 | 4.41 |
| | Se77 | 7.60 | 8.29 | 8.67 |
| | Se78 | 23.60 | 23.27 | 25.89 |
| | Se80 | 49.70 | 49.70 | 49.70 |
| | Se82 | 9.20 | 0.72 | 10.42 |
| Si | Si28 | 92.23 | 96.48 | 89.84 |
| | Si29 | 4.67 | 0.85 | 4.04 |
| | Si30 | 3.10 | 2.67 | 6.12 |

The study of essential elements and their isotope ratios in various cells and hormones provides strong indications that the red bone marrow, organs, and cells producing signaling molecules and hormones can separate isotopes. This makes it possible to enrich critical cells and molecules with light isotopes, to ensure that polypeptides and proteins have the perfect conformation. The negative aspect of this function is the accumulation of the waste-heavy isotopes in nearby tissues. These local sites act as a feeding ground for the onset of all types of pathologies.

No wonder—the map of both primary tumors and metastases perfectly coincides with the map of immune and endocrine systems and location of cells producing signaling molecules.

Receptors with damaged conformation protect pathological cells from immune system recognition cells. It seems that by accumulating heavy isotopes and D-amino acids the cancer cell-“the emperor of malignancy”-attempts to become an invincible mirror image cell. This is not the only means of protection that cancer cells deploy. It has been discovered that the tissue around metastases is enriched with light isotopes. Metastases suck out heavy isotopes from the surrounding tissue, making it appear as an extremely innocuous environment, where immune system cells would never attack the metastases.

Accumulation of heavy isotopes and D-amino acids are hallmarks of every degenerative disease and aging. This makes them strong and capable of defeating life, but it also makes them extremely vulnerable.

Pathology cannot progress absent heavy isotopes. The way to fight them is by changing “isotope ratios” of essential elements in favor of atoms that have an equal number of protons and neutrons. The ideal therapeutic composition will include a mixture of the lightest isotopes of all essential elements in each of the amount proportional to the correspondent daily consumption dose. This approach should be sufficient to correct the structure of the metalloproteins but not of all proteins. To make sure that all proteins are produced without mistakes one must address all stages of their synthesis and assembly: transcription, translation, and posttranslational modification. Living cells use several mechanisms to repair damaged DNA and mitigate problems related to mutant and misfolded proteins. There are several repair mechanisms involving other proteins, which must also be functional and responsible for facilitating the correct conformational assembly and catalytic activity for each protein which maintains homeostasis within the human body.

In general, all proteins including transcription factors are produced in ribosomes. Ribosomes synthesize peptides from amino acids according to the instruction of messenger RNAs and are one of the most important molecular machines of cellular life. They are also responsible for conformational changes within proteins. The quality of ribosomes defines the quality of proteins. A ribosome is composed of a large subunit and a small subunit, together consisting of three to four ribosomal RNAs (rRNAs) and dozens of ribosomal proteins (r-proteins). Common features in ribosomal proteins are zinc finger motifs, which are associated with the proteins of both subunits of the ribosomes. Ribosomal proteins play a major role as RNA-binding proteins. Success of the translation process hinges on proper binding of zinc finger proteins and therefore, on their conformation. The average lifetime of ribosomes is 4-6 minutes. They are rapidly produced in autocatalytic reactions. Autocatalytic reactions may amplify the yield of proteins with the wrong conformation induced by isotope substitution of heavy isotopes. The probability of these events is quite low for non-metalloproteins, as their atoms light isotopes ratio is close to 100% (H-1, 99.98%; C-12, 98.89%; 0-16, 99.76% and N-14, 99.63%).

The situation is completely different in case of zinc finger proteins. Isotope ratio for the stable isotope with minimum excess of neutrons over protons Zn-64 is just 48, 60%; therefore the probability of substitution by heavier isotopes is remarkably high. The situation could be negatively impacted by asymmetric autocatalysis. It renders the probability of the resulting mistranslation in all produced proteins extremely high. Zinc is recognized as one of the most essential elements for the human body. Numerous scientific articles have documented the effect of excess or lack of zinc on human health. The influence of zinc is mentioned in relation to at least 60 diseases. However, the most important issue is which precise zinc of the five stable isotopes—and it is not addressed in scientific literature. It is this issue that is fundamental to the proper operation of the ribosome and therefore, for synthesis of proteins with the correct conformation.

In immune system cells produced by the red bone marrow, as well as in signaling molecules produced by the endocrine system, the isotope ratio of light isotope Zn-64 significantly exceeds natural abundance. Conversely, a much higher concentration of heavy zinc isotopes is observed in the cells of pathological tissues. A short-term or long-term increase in the concentration of light zinc isotopes in the cytoplasm of cells will ensure their access to the ribosomes. This will lead to a sharp increase in the proportion of proteins with the correct conformation produced by ribosomes. The goal is to replace all damaged/old biomolecules, including all receptors and signaling molecules with new/young ones featuring light isotopes.

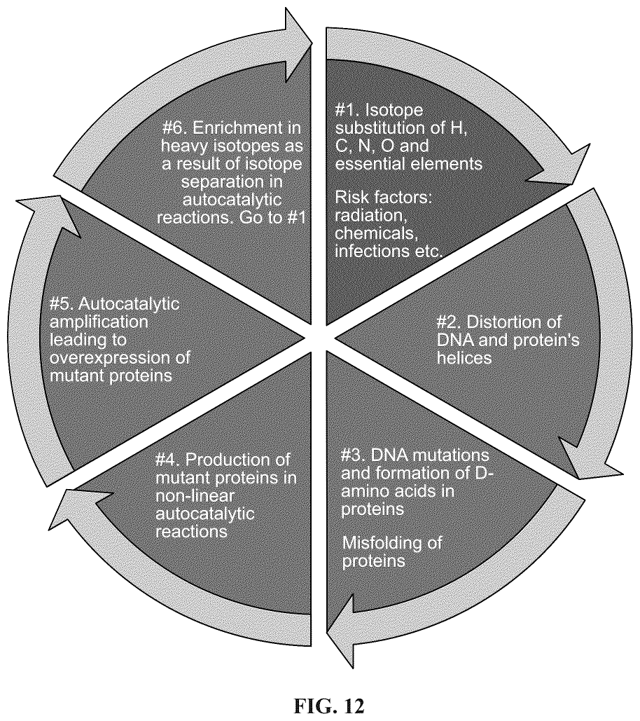

The conclusion about the influence of the isotopic composition of chemical elements on the homeostasis of biological systems suggests a paradigm shift in the understanding of the nature of pathogenesis (FIG. 12). WO2022203684A1.

The mechanism of degenerative diseases can be summarized as following:
1. Heavy isotope substitution occurs naturally through aging and certain disease processes.
2. This induces distortion of helices in DNA and proteins (including Parkin, p53 and Ribosomes which have zinc fingers) A. A. Ivanov. Russian journal of physical chemistry B, v.2, No 6, pp. 649-652, 2007.
3. DNA defects drive disrupted synthesis and of chirality of Amino Acids (AAs) in proteins.
4. Amplified yields of the “wrong”, misfolded, or otherwise mistranslated proteins by autocatalysis leading to their overexpression.
5. “Wrong” proteins require heavy isotopes causing a reverse process of isotope separation in autocatalytic reactions.
6. “Wrong” proteins drive further intake of heavy isotopes, repeating the process.
7. This creates a positive feedback loop that drives a cascade of injury in neurons, which are extremely sensitive to misfolded proteins, thereby driving degeneration.

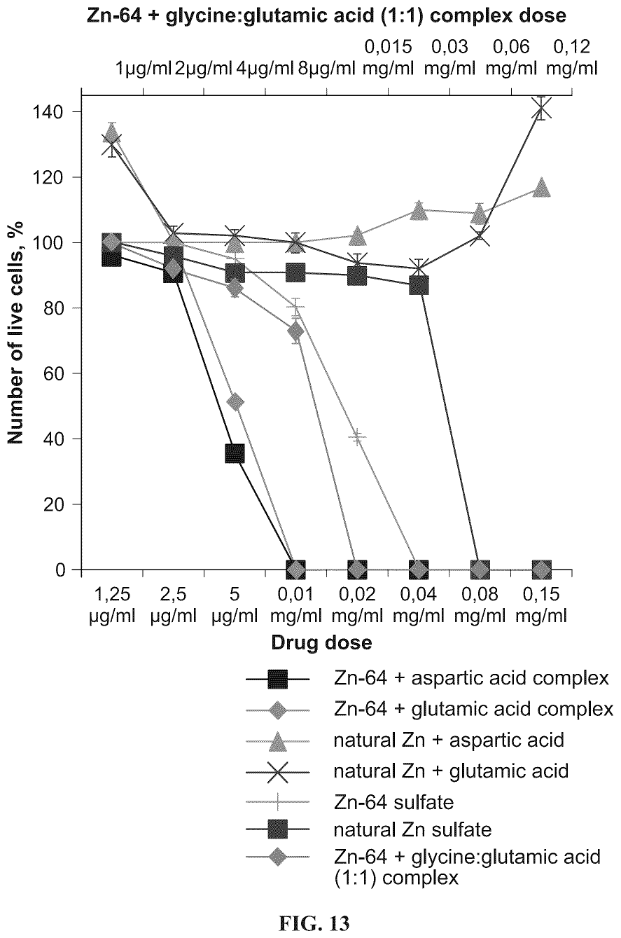

In this regard, an illustrative example of the opposite effect of natural zinc and a stable isotope Zn-64 on the proliferation of cancer cells is quite enlightening, as noted in the data in FIG. 13.

Therapeutic compositions that increase the isotope ratio of Zn-64 and lightest isotopes of other essential elements in the cytoplasm of living cells is a completely new family of drugs of general effect. Such drugs create conditions incompatible with life for cells affected by the most serious pathologies, such as cancer. U.S. Pat. No. 10,857,180; U.S. patent application Ser. Nos. 16/236,343; 16/973,169; PCT/US19/55770; PCT/US21/24433.

The use of such therapeutic compositions containing the lightest isotopes of essential chemical elements is helpful but not sufficient to ensure complete recovery from degenerative diseases, restoration of homeostasis that is typical for a young and healthy organism, and to renew immune and endocrine systems, and rejuvenate live cells.

Yet problems remain:
1. Even in the lightest isotopes of the most of chemical elements there is still an excess of neutrons over protons in the nucleus.
2. An increase in the relative concentration of “therapeutic” light isotope in the cell cytoplasm does not in any way affect and therefore, does not decrease the absolute content of heavier isotopes of this chemical element in the same cell.

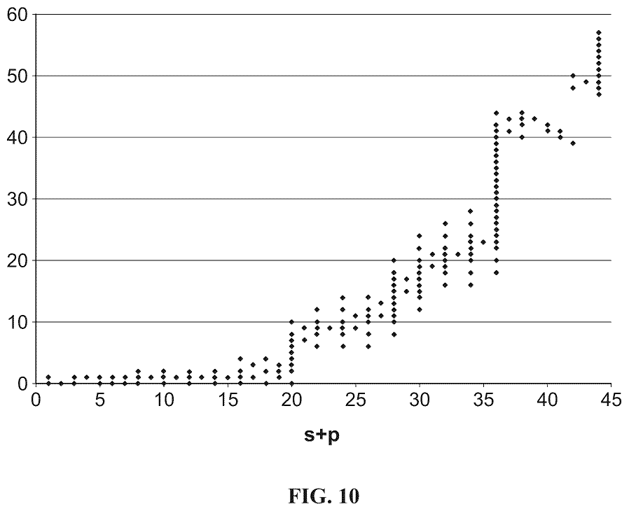

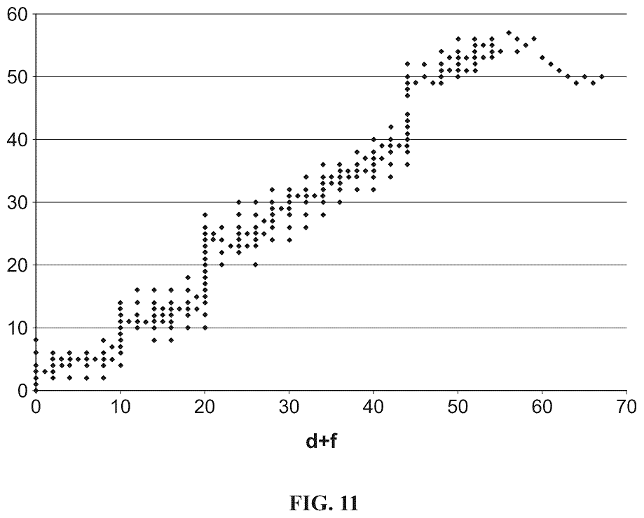

For example, the nucleus of the lightest zinc isotope Zn-64 has 30 protons and 34 neutrons with an excess of neutrons over protons equal to four. The addition of Zn-64 to the cytoplasm of the cell, being a positive factor with therapeutic effect, cannot reduce the concentration of heaviest isotopes Zn-68 and Zn-70 already present in the cell. In fact, a similar situation is typical for all non-monoisotopic chemical elements. The excess of neutrons over protons is a serious problem affecting the efficacy of therapeutic compositions based on the lightest isotopes of chemical elements. As can be seen from the graphs in FIG. 9, FIG. 10, and FIG. 11, there is a directly proportional relationship between the sum of s- and p-electrons, sum of d- and f-electrons and excess of neutrons over protons. This dependence is found starting from the s+p=19 (FIG. 10), that is from potassium. Given the earlier conclusion that potassium is in fact a d-element, one concludes that the isotope composition of chemical elements with d- and f-outer electrons can affect the balance between potential and solenoidal components of a self-sustained oscillating system of polarization potential and the same type of components in the newly formed biomolecule and especially those containing chemical elements with atomic number equal or higher than 19. Given the low energy of formation of a solenoidal components/helix of DNA and proteins, it can be expected that the effect will be the greater the higher the value of the sum (d+f). As mentioned above, here it is meant an energy level of the order 0.01 eV. In fact, quite high values of (d+f) can lead to a violation of negative feedbacks with incomparably higher energy of the order of +/−1 MeV and even cause the decay of atoms. This conclusion can be drawn from the analysis of the nature of parabolas corresponding to the 4f-electron states in FIG. 9. For chemical elements with the atomic number above 83, the energies of 4f-electron states not only significantly exceed the energies of 5d-, 6s-, 6p- and 5f-states, but also are comparable if not with the energies of strong interaction, then at least with the energy released at neutron decay. It makes sense to take this factor into account when analyzing the conditions for the radioactive decay of chemical elements with the atomic number higher than that of bismuth.

It is known from the course of nuclear physics that, because of radioactive decay, atoms with a smaller excess of neutrons over protons are formed and which are much more stable.

It makes sense to draw an analogy with the division of live cells. For the reasons and due to the processes described earlier, live cells in the process of life are enriched with heavy isotopes. This plays a key role in the process of pathogenesis. The Gi phase of cell division is much shorter than the cell's lifetime. Therefore, cell division makes it possible to reduce the total excess of neurons over protons twofold, and, as a result, increase stability. Cancer cells are enriched in heavy isotopes much more strongly than normal ones. Therefore, to ensure stability, they divide much more often and not only into two cells, but also into four cells. Neurons do not divide and therefore are subject to accelerated aging and eventually die.

Therefore, an increase in the concentration of lightest isotopes of essential chemical elements in the cytoplasm of live cells is a therapeutic solution to combat degenerative diseases and aging. U.S. Pat. Nos. 10,857,180 and 11,286,236; WO2021071499A1; WO2022203684A1.

Considering the role of autocatalytic reactions in the amplification of chirality of biomolecules and in the separation of isotopes, one could expect a significant change in the concentration of precisely that isotope that is additionally introduced into the cytoplasm.

Moreover, the therapeutic dose in this case plays the role of ignition. Subsequently, additional light isotopes can be extracted from the blood supply.

It is important to emphasize that the separation of isotopes during autocatalytic reactions requires that these isotopes be an integral part of biomolecules. However, mass-spectroscopy data obtained disagrees with this conclusion:
1. Pathology affected tissues are enriched with heavy isotopes of all essential elements including those that never have been found to be an integral part of biomolecules or bound to them (K and Rb, for example). Table 3.
2. Cells of immune system, sperm, breast milk are enriched with light isotopes of all essential elements including those that never have been found to be an integral part of biomolecules or bound to them (K and Rb, for example). Table 4.
3. Use of a light isotope of a one specific chemical element (Zn-64) as a therapy translates into the shift in the isotope ratios in favor of light isotopes for all essential elements (Table 5).

TABLE 3
Isotope ratios in cancer cells and tissues. U.S. Pat. Nos. 10857180 and 11286236;
WO2021071499A1; WO2022203684A1

| | | | | | | Tissue |
|---|---|---|---|---|---|---|
| | | | | | | Around |
| | | | | | Metastasis | Metastasis |
| | | | | | of | of |
| | | | Lymphocytes | Intestinal | Intestinal | Intestinal |
| | | Abundance | Leukemia | Tumor | Tumor | Tumor |
| Element | Isotope | Natural 8 | Avg. % | Avg. % | Avg. % | Avg. % |
| Zn | Zn64 | 48.60 | 45.59 ± 0.66 | 46.49 ± 0.35 | 40.59 ± 0.15 | 55.62 ± 2.51 |
| | Zn66 | 27.90 | 25.44 ± 0.7 | 24.66 ± 2.64 | 25.5 ± 0.28 | 25.22 ± 1.84 |
| | Zn67 | 4.10 | 3.85 ± 0.04 | 4.82 ± 0.1 | 3.85 ± 0.04 | 3.93 ± 1.0 |
| | Zn68 | 18.80 | 23.77 ± 1.27 | 20.42 ± 0.14 | 23.77 ± 1.27 | 14.63 ± 1.24 |
| | Zn70 | 0.60 | 1.42 ± 0.21 | 3.63 ± 0.2 | 6.29 ± 0.2 | 0.60 |
| Fe | Fe54 | 5.80 | 4.07 ± 0.46 | 4.44 ± 1.25 | 3.07 ± 0.02 | 6.43 ± 0.21 |
| | Fe56 | 91.72 | 91.4 ± 0.24 | 91.29 ± 0.38 | 91.4 ± 0.24 | 91.13 ± 0.19 |
| | Fe57 | 2.20 | 4.25 ± 0.61 | 2.16 ± 0.12 | 5.25 ± 0.3 | 2.16 ± 0.06 |
| | Fe58 | 0.28 | 0.28 | 2.11 ± 0.03 | 0.28 | 0.28 |
| Mg | Mg24 | 78.99 | 76.53 ± 0.46 | 78.47 ± 0.24 | 74.19 ± 0.88 | 80.95 ± 0.6 |
| | Mg25 | 10.00 | 7.57 ± 0.19 | 6.47 ± 0.3 | 9.57 ± 0.18 | 9.57 ± 0.44 |
| | Mg26 | 11.01 | 15.93 ± 0.5 | 15.06 ± 0.76 | 16.23 ± 0.29 | 9.39 ± 0.64 |
| Si | Si28 | 92.23 | 89.84 ± 0.57 | 90.84 ± 0.39 | 87.71 ± 0.84 | 92.73 ± 0.1 |
| | Si29 | 4.67 | 4.04 ± 0.25 | 4.49 ± 0.24 | 3.38 ± 0.6 | 4.51 ± 0.1 |
| | Si30 | 3.10 | 6.12 ± 0.17 | 4.67 ± 0.3 | 8.91 ± 0.71 | 2.76 ± 0.09 |
| Se | Se74 | 0.90 | 0.90 | 0.90 | 0.90 | 0.90 |
| | Se76 | 9.00 | 4.41 ± 0.23 | 7.73 ± 0.58 | 3.51 ± 0.37 | 10.84 ± 2.32 |
| | Se77 | 7.60 | 8.67 ± 0.7 | 3.33 ± 0.21 | 7.33 ± 0.37 | 7.97 ± 0.85 |
| | Se78 | 23.60 | 25.89 ± 2.64 | 25.98 ± 1.72 | 25.89 ± 2.64 | 22.32 ± 1.53 |
| | Se80 | 49.70 | 49.70 | 50.61 ± 0.33 | 50.13 ± 1.28 | 49.70 |
| | Se82 | 9.20 | 10.42 ± 0.55 | 11.46 ± 0.16 | 12.25 ± 0.61 | 8.27 ± 0.88 |
| Rb | Rb85 | 72.16 | 68.19 ± 1.33 | 70.45 ± 0.22 | 65.19 ± 0.23 | 73.7 ± 0.38 |
| | Rb87 | 27.84 | 31.81 ± 0.7 | 29.55 ± 0.22 | 34.81 ± 0.18 | 26.3 ± 0.38 |

TABLE 4
Isotope ratios of normal cells, breast milk and sperm, U.S. Pat. Nos. 10857180 and 11286236;
WO2021071499A1; WO2022203684A1

| | | | | | | | | PL of |
|---|---|---|---|---|---|---|---|---|
| | | Natural | | Breast | Healthy | | | Healthy |
| | | Abundance | Sperm | Milk | Lymphocytes | Erythrocytes | Thrombocytes | Blood |
| Element | Isotope | % | Avg. % | Avg. % | Avg. % | Avg. % | Avg. % | Avg. % |
| Zn | Zn64 | 48.60 | 63.34 ± 0.89 | 53.58 ± 0.22 | 58.83 ± 0.79 | 56.64 ± 1.33 | 55.00 ± 1.11 | 59.1 ± 0.46 |
| | Zn66 | 27.90 | 28.07 ± 0.51 | 27.52 ± 0.34 | 30.88 ± 1.39 | 27.6 ± 0.81 | 31.38 ± 5.56 | 27.62 ± 1.51 |
| | Zn67 | 4.10 | 4.25 ± 0.26 | 3.89 ± 0.08 | 3.74 ± 1.33 | 3.66 ± 0.24 | 4.25 ± 1.56 | 3.71 ± 0.98 |
| | Zn68 | 18.80 | 3.81 ± 0.39 | 14.96 ± 0.56 | 5.95 ± 0.29 | 11.99 ± 0.01 | 8.77 ± 0.45 | 8.97 ± 0.63 |
| | Zn70 | 0.60 | 0.57 ± 0.1 | 0.05 ± 0.03 | 0.60 | 0.11 ± 0.03 | 0.60 | 0.60 |
| Fe | Fe54 | 15.80 | 11.64 ± 0.57 | 5.99 ± 0.1 | 11.17 ± 0.68 | 10.4 ± 0.49 | 8.68 ± 0.23 | 6.14 ± 0.17 |
| | Fe56 | 91.72 | 87.78 ± 1.15 | 93.66 ± 0.08 | 87.55 ± 0.77 | 88.64 ± 0.71 | 90.89 ± 0.38 | 93.6 ± 0.22 |
| | Fe57 | 2.20 | 0.26 ± 0.03 | 0.07 ± 0.03 | 0.98 ± 0.51 | 0.93 ± 0.03 | 0.15 ± 0.14 | 0.04 ± 0.03 |
| | Fe58 | 0.28 | 0.28 | 0.28 | 0.28 | 0.01 | 0.28 | 0.22 |
| Mg | Mg24 | 78.99 | 83.04 ± 1.15 | 83.81 ± 0.97 | 83.65 ± 0.89 | 85.72 ± 0.44 | 82.1 ± 1.39 | 83.99 ± 0.59 |
| | Mg25 | 10.00 | 10.72 ± 0.21 | 11.13 ± 0.33 | 9.57 ± 0.87 | 8.92 ± 0.44 | 9.57 ± 1.32 | 9.52 ± 0.46 |
| | Mg26 | 11.01 | 6.38 ± 0.8 | 5.06 ± 0.04 | 6.77 ± 0.6 | 5.36 ± 0.86 | 7.45 ± 1.18 | 6.49 ± 0.25 |
| Si | Si28 | 92.23 | — | 93.41 ± 0.22 | 96.48 ± 0.71 | 93.66 ± 0.31 | 92.07 ± 0.63 | 94.64 ± 0.76 |
| | Si29 | 4.67 | — | 4.78 ± 0.17 | 0.85 ± 0.3 | 3.49 ± 0.29 | 4.77 ± 0.44 | 4.85 ± 0.34 |
| | Si30 | 3.10 | — | 1.81 ± 0.77 | 2.67 ± 0.15 | 2.85 ± 0.48 | 3.16 ± 0.33 | 0.51 ± 0.27 |
| Se | Se74 | 0.90 | — | 0.90 | 2.73 ± 0.81 | 3.62 ± 0.42 | 0.90 | 0.90 |
| | Se76 | 9.00 | — | 14.35 ± 0.69 | 15.29 ± 1.28 | 11.67 ± 0.46 | 14.9 ± 0.8 | 16.29 ± 1.04 |
| | Se77 | 7.60 | — | 6.37 ± 1.14 | 8.29 ± 0.98 | 8.13 ± 0.66 | 7.54 ± 0.76 | 6.79 ± 1.14 |
| | Se78 | 23.60 | — | 24.49 ± 1.27 | 23.27 ± 1.18 | 24.81 ± 1.15 | 20.39 ± 0.24 | 23.65 ± 1.72 |
| | Se80 | 49.70 | — | 46.70 | 49.70 | 44.14 ± 0.83 | 49.70 | 44.53 |
| | Se82 | 9.20 | — | 7.19 ± 0.93 | 0.72 ± 0.16 | 7.63 ± 1.02 | 6.57 ± 1.09 | |
| Rb | Rb85 | 72.16 | 90.95 ± 0.78 | 77.01 ± 0.89 | 92.12 ± 0.82 | 77.47 ± 0.28 | 91.53 ± 2.02 | 77.16 ± 0.18 |
| | Rb87 | 27.84 | 9.04 ± 0.03 | 22.99 ± 0.89 | 7.88 ± 0.82 | 22.53 ± 0.49 | 8.47 ± 2.02 | 22.84 ± 0.56 |

TABLE 5
Effect of 10 I.V. daily Zn-64 aspartate injections on
isotopes ratios in mice brains six samples per brain.
U.S. Pat. Nos. 10857180 and 11286236;
WO2021071499A1; WO2022203684A1

| | Control/ | | | | | | |
|---|---|---|---|---|---|---|---|
| | No Injections | | | Natural | After Injections | | |
| | Min | Max | Average | Abundance | Average | Max | Min |
| Zn64 | 49.07 | 54.94 | 53.12 | 48.60 | 58.44 | 58.99 | 57.87 |
| Zn66 | 24.06 | 25.06 | 24.14 | 27.90 | 28.44 | 28.55 | 28.21 |
| Zn67 | 3.10 | 4.09 | 3.86 | 4.10 | 1.04 | 1.22 | 0.94 |
| Zn68 | 15.22 | 17.32 | 16.28 | 18.80 | 11.48 | 11.74 | 11.32 |
| Zn70 | 0.55 | 0.66 | 0.60 | 0.60 | 0.60 | 0.75 | 0.46 |
| Mg24 | 78.29 | 81.72 | 80.13 | 78.99 | 82.50 | 83.95 | 81.32 |
| Mg25 | 8.13 | 10.86 | 9.96 | 10.00 | 8.94 | 9.48 | 8.35 |
| Mg26 | 9.22 | 11.24 | 10.21 | 11.01 | 8.56 | 8.79 | 8.32 |
| Fe54 | 5.78 | 6.85 | 6.14 | 5.80 | 7.95 | 8.85 | 7.34 |
| Fe56 | 91.26 | 91.81 | 91.55 | 91.72 | 91.99 | 92.68 | 91.61 |
| Fe57 | 1.80 | 2.20 | 2.02 | 2.20 | 0.22 | 0.55 | 0.00 |
| Fe58 | 0.28 | 0.28 | 0.28 | 0.28 | 0.28 | 0.28 | 0.28 |
| K39 | 89.45 | 92.46 | 90.89 | 93.26 | 95.50 | 96.80 | 94.45 |
| K41 | 8.36 | 10.04 | 9.11 | 6.74 | 4.50 | 4.99 | 4.16 |
| Rb85 | 69.42 | 71.80 | 70.81 | 72.16 | 76.66 | 76.78 | 76.42 |
| Rb87 | 28.22 | 29.58 | 29.19 | 27.84 | 23.34 | 23.82 | 23.01 |
| Si28 | 91.62 | 94.21 | 92.64 | 92.23 | 93.40 | 93.88 | 92.27 |
| Si29 | 3.81 | 4.84 | 4.46 | 4.57 | 4.36 | 4.57 | 4.02 |
| Si30 | 3.01 | 3.09 | 2.88 | 3.10 | 2.24 | 2.83 | 1.82 |

Based on the above-mentioned theoretical model of stable systems and these experimental results, two important conclusions can be drawn.
1. Molecular signatures like overexpression of certain proteins, disfunction of specific pathways, mutations of DNA and changes of proteins conformation, other abnormalities and even isotope ratios of essential elements are just symptoms of fundamental changes in the balance of differential and integral components of vector fields, the most important of which is the polarization potential field, at least at the level of biomolecules, live cells, and tissues.
2. In previous patents and patent applications, two extreme cases were used, based on the biological effects of the lightest and most heavy isotopes of essential elements. It turns out that the balance of integral and differential components of the vector function of polarization potential in a live cell is the result of the total simultaneous action of all isotopes of all essential chemical elements and especially d- and f-elements in accordance with the disclosed classification.
3. This opens new possibilities for enhancing the therapeutic effect of drugs based on the use of light isotopes of essential chemical elements. The important but not the only element in the realization of these possibilities is the therapeutic composition with minimized excess of neutrons over protons and capable to serve as a source of proper/healthy vector fields in a live cell.

Now the essence of a technical solution to create a new type of therapeutic drug can be outlined. However, before doing this, it makes sense to look at the problem under discussion differently.

The theory and general principles of the formation of stable systems are presented herein. Then these principles were used to describe the atom as a self-sustained oscillating system of polarization potential which agrees with experimental data presented further. In doing so, the so-called electric polarization potential is used. Mathematics and the scheme in FIG. 1 tell that in any stable system both potential and solenoidal components should be present. Strictly speaking, this means that it makes sense to consider two separate cases. The first case only for electric polarization and the second case solely for magnetic polarization. Then, considering the generality of the scheme in FIG. 1, one should get an analogue of the scheme in FIG. 2, but already for the case of magnetic polarization, which describes the oscillations of the magnetic charge density. In this case the atom as two interconnected auto-oscillating systems must be considered. The first is associated with oscillations of the density of the electric charge of electrons and protons, and the second with oscillations of the density of the magnetic charge of the magnetic analogs of electrons and protons. Both systems are interconnected through the differential characteristic of polarization potential-vector potential:

$$\begin{matrix}A = μϵ \frac{\partial }{\partial t} \\ A = ∇ \times \end{matrix}$$

The problem is that, according to Maxwell's equations, magnetic charges do not exist.

$$\begin{matrix}∇ \cdot E = \frac{{ρ}_{e}}{{ε}_{0}} \\ ∇ \cdot B = 0\end{matrix}$$

Even though Maxwell's equations do not require complete symmetry of electric and magnetic interactions, the existence of magnetic charge would explain the quantization of electric charge as shown by Dirac. Paul Dirac, “Quantized Singularities in the Electromagnetic Field”. Proc. Roy. Soc. (London) A 133, 60 (1931). Scientists are constantly searching for magnetic monopoles in various energy ranges from extremely high to a few GeV and lower. L. D. Landau and E. M. Lifshitz, Course of Theoretical Physics, Vol. 2: The classical theory of fields (Nauka, Moscow, 1973; Pergamon, Oxford, 1975), p.91; A. D. Sakharov, JETF Lett. 44, 379 (1986). Ya. B. Zeldovich and M. Yu. Khlopov, Phys. Lett. B, 79, 239 (1978); J. Schwinger, Phys. Rev. D 12, 3105 (1975).

Some experts in theoretical physics argue that it is impossible to observe isolated magnetic charges (monopoles). Others admit the possibility of the existence of magnetic charges, especially in the context of the early Universe. They suggested that cosmological heavy monopoles were immediately bound into a magnetic atom with the size three order of magnitude smaller than Bohr radius of the hydrogen atom. According to Schwinger, the relation between electric and magnetic charges is g=137e. It leads to the minimum mass of the magnetic charge in Schwinger's symmetric world m_{g}=(g/e)²mₑ or approximately 18769 mₑ=10.24mₚ≈9.6 GeV/c². J. Schwinger, Phys. Rev. D 12, 3105 (1975).

This value of energy is so much bigger than the energy of neutron decay (0.782343 MeV) that it makes quite impossible to consider the notion of magnetic charges to be relevant to the disclosed model.

However, there is an important feature that neither Dirac (Paul Dirac, Proc. Roy. Soc. (London) A 133, 60 (1931) nor Schwinger (J. Schwinger, Phys. Rev. D 12, 3105 (1975)) considered. These scientists, as well as their followers, consider the world in which Maxwell's equations would be symmetric. In other words, a world in which both electric and magnetic charges exist simultaneously. Dirac was considering the flux of the 6-vector E, H.

Accordingly, all formulas that have a relationship between the electric and magnetic charges, of quantization of the electric charge, the assessment of the energy of magnetic monopole are based on the idea of the simultaneous existence of electric and magnetic charges in a certain point of space (x,y,z). This is possibly the reason for the futility of attempts to experimentally detect magnetic monopoles. Therefore, the power of modern accelerators is not enough to hit collide several particles and detect the birth of magnetic charges. Another suitable explanation is that yes, now the magnetic monopoles do not exist, but they could well have existed in the early Universe. Modern science, including theoretical physics, bypasses the question of stability of natural objects and the need for the existence of negative feedback to ensure stability. This applies to the nature of chemical bonds, electromagnetic interaction, strong and weak interactions and even to the Grand Unified Theory.

From the point of view of modern paradigm, the answer to the question of where to look for magnetic monopoles can sound like this: in space-time continuum, in an empty space, in the vacuum, in the atom, in the nucleus. Time component may also be added: at present or long ago. The problem is that these questions and answers are irrelevant. Based on the physical meaning of vector function and general scheme/plan/algorithm of the stable self-sustained system (FIG. 1) the only question to be asked: “where? In the divergence free or curl free space?” Bearing in mind the model of the atom (FIG. 2), one may ask: “where? In the space of electric polarization potential or in the space of magnetic polarization potential?”. Then, the very issue of simultaneous existence/presence of electric and magnetic charges simply disappears.

First, because vector fields of electric and magnetic polarization potentials are orthogonal. Second, because these fields must be considered in terms of primary and secondary or in terms of integral and differential components with respect to time and distance.

Dirac's assumptions mean that six-vector exists in the world. In terms of polarization potential, it means that both electric polarization potential and magnetic polarization potential are present simultaneously in the world. Where exactly in the space we live in? Where are exactly magnetic charges (not Dirac monopoles) not found? The correct answer is everywhere outside of nucleus. And what it means “outside of nucleus”. The simplest answer is “in the world where Maxwell's equations are correct” and where magnetic charges are found and, in the world, where electrons and protons with opposite electric charges are real. In other words, in the space where there is no magnetic polarization potential. Then, what about inside of the nucleus?

Composition of nucleus is known due to the study of the particles coming from the nucleus. Coming to where? To the field of Me. But suppose that there are two components of atom: vector field of \vec{Π}e and vector field of \vec{Π}m and they are orthogonal, then particles which belong to magnetic polarization potential field are not supposed to be stable in \vec{Π}e field/space. It is well known that neutron lifetime in \vec{Π}e field is about 14 minutes only, and then it decays. Particles from the \vec{Π}m field are not able to remain stable in field/space where \vec{Π}m=0. They should decay and take the form different from the one they used to have in the magnetic polarization potential field. With this logic one can consider neutron as \vec{Π}m field analog of hydrogen from the \vec{Π}e field. The estimate of the size of the nucleus carried out based on a comparison of the energy released during the decay of the neutron and ionization potential of the electron in the atom of hydrogen supports this hypothesis. Hydrogen atom and neutron are simplest limiting cases.

Then the rest of the atoms can be represented as consisting of two localized and electrically neutral auto oscillating system of the electric and magnetic polarization potentials \vec{Π}e and \vec{Π}m interconnected through the function of vector potential A.

The relationship through the vector potential A does not jeopardize the idea of orthogonality between two types of polarization, since the vector potential is a differential component of both \vec{Π}e and \vec{Π}m.

The first component describes the oscillations of the electric charge density between protons and electrons as auto oscillating system of electric polarization potential. The model of this system described herein above provides benefits, including rules for filling electron orbitals, identical to ones of the quantum mechanical model of the atom, which was completed prior to the discovery of the neutron.

The second component describes the oscillations of the magnetic charge density between \vec{Π}m analogs of protons and \vec{Π}m analogs of electrons as auto oscillating system of magnetic polarization potential.

Here notion of magnetic monopoles is not used since some of Dirac's assumptions leading to estimation of mass, charge, and energies of interaction between magnetic particles may not be correct.

Therefore, it is quite logical to use Pauli exclusion principle analog to describe the structure of the second \vec{Π}m—component of the atom in terms of energy levels and \vec{Π}m analogs of electrons configurations.

Solely for the purpose of simplification the \vec{Π}e component is named as an “electric” one and \vec{Π}m component is named as a “magnetic” one.

The shells of electric and magnetic components are independent of each other for as long as energies of negatively charged particles in \vec{Π}e component and \vec{Π}m component are not the same. As follows from the data in FIG. 9, appearance of a degenerate state causes the decay of an atom.

At this point, one can see a slight similarity between the disclosed model and nuclear shell model describing the energy level arrangement of protons and neutrons in the nucleus in terms of energy levels. This model allows one to imagine building a nucleus by adding protons and neutrons according to the same rules that are used by quantum mechanics to explain filling of electron shells. According to this model nucleon moves in some effective potential created by forces of all other nucleons. Gapon E., Iwanenko D., Zur Bestimmung der isotopenzahl, Die Naturwissenschaften, Bd.20, s.792-793, 1932. This model is considered relevant at the present time.

The difference is that in this case, the sources of the fields are well defined. The description of the “electric component” is fully consistent with the basics of quantum mechanics and in addition explains the nature of negative feedbacks that ensure the stability of the atom.

There is no need to question the theory of the short-range strong interaction here. It acts at the range of 10⁻¹⁵ m and is not able to affect chemical bonds formation of chirality of biomolecules.

On the other hand, the effect of isotope induced chirality discovered by Kenso Soai et al. (Kenso Soai. Amplification of chirality. Springer Berlin Heidelberg, 2010, 205 p.; Guillermina Losano, Arnold J. Levine, Ed. The p53 protein form cell regulation to cancer. Cold Spring Harbor Perspectives in Medicine; Kawasaki T. et al., Science 2009, 324, 492-495; Kawasaki T. et al., Chem. Int. Ed. 2011, 50, 8131-8133; [Angew. Chem. 2011, 123, 8281-8283; Arimasa Matsumoto et al., 2016 Dec. 5; 55 (49): 15246-15249) and opposite biological effect of light and heavy isotopes used described in several patent descriptions unambiguously prove that nucleus/isotope composition do affect biomolecules and live cells. It is important and requires better understanding.

Comparison of the energy released during decay of the neutron (0.78 MeV) and ionization potential of the electron in the atom of hydrogen (13.6 eV) indicates that the geometric dimensions of the magnetic component are at least four to five orders of magnitude smaller than the electric one. Nevertheless, despite the small geometric size, the “magnetic component” is about electromagnetic interaction, not the strong one, and therefore, affects the formation and stability of helices in bio molecules.

The facts already mentioned above together with a newest one in item four make proof that biological processes in live cells are defined by vector fields.
1. Formation of complex hierarchical biomolecules in picoseconds time frame.
2. The shift of zinc isotopes ratio in favor of Zn-64 induces change increase in values of lightest isotopes ratios in the rest of essential chemical elements (Table 5).
3. Plasma dust experiments data; V N Tsytovich et al., New Journal of Physics 9 (2007) 263; H M Thomas et al., New Journal of Physics, Volume 10, March 2008
4. Latest experiments demonstrating sudden release of billions of atoms of zinc (probably heavy isotopes of zinc) and intake of billions of atoms of calcium by the cells right in the moment of fertilization. Marla Paul. Radiant Zinc Fireworks Reveal Quality of Human Egg. Discovery could help fertility doctors decide best eggs to implant for IVF. https://news.northwestern.edu/stories/2016/04/radiant-zinc-fireworks-reveal-quality-of-human-egg

Theory and experimental data discussed indicate that such fields are integral and differential components of the electric polarization potential and magnetic polarization potential. The sources of these fields are the chemical elements from which biomolecules are made. Cellular homeostasis/negative feedbacks and, accordingly, the health of the cells depend on the balance of these fields. An excess of the number of neutrons over the number of neutrons in the chemical elements means the excess of the magnetic component over the electric one. The stability of a live cell, as well as the stability of atoms, the higher the smaller the difference between number of neutrons and protons in chemical elements. In this case, it is not so much the number of protons and neutrons that matters as the balance between fields of electric and magnetic polarization potentials. Since the resulting vector field is the sum of the separate contributions of \vec{Π}ₑ and \vec{Π}ₘ, it is this balance that dictates the required concentration of each chemical element inside a live cell.

By mass, about 96% of our bodies are made of four key elements: oxygen (65%), carbon (18.5%), hydrogen (9.5%) and nitrogen (3.3%). In all of them number of protons is practically equal to the number of neutrons (Table 1), Phosphorus, constituting 0.7% of body mass, is a monoisotopic element. Calcium—the most abundant chemical element in our body takes another 1.5% of mass. Isotope ratio of lightest isotope Ca-40 with equal number of protons and neutrons is 96, 9418. This means that the balance between \vec{Π}ₑ and \vec{Π}ₘ in live cells is defined exclusively by the essential chemical elements constituting just 1.58 of the body mass. Not all these elements are equally important. First, one needs to take care of the chemical elements that are part of metalloproteins, and of elements whose ions perform a signaling function. The following elements are commonly used in food supplements: K, Ca, Sc, Ti, V, Cr, V, Cr, Mn, Fe, Co, Ni, Cu, Zn, Se, Mo, Sn. Usually, research studies the role of a shortage or excess of essential chemical elements on the biological functions of the body and tries to establish a correlation with one or another type of degenerative disease. Literary data on the effectiveness of using the essential elements for therapeutic purposes are very contradictory. The inconsistency is due to the opposite biological effects of light and heavy isotopes of the same element.

Compositions based on the lightest isotopes of essential elements have therapeutic effect; but may not be a cure. Clinicians would say that drug efficacy is not sufficient, and the therapeutic dose is higher than maximum tolerated dose. Increase the dose higher than MTD (maximum therapeutic dose) is impossible, and this situation becomes dead end.

The use of a combination of light isotopes of several chemical elements does not allow reducing the therapeutic dose and does not lead to the solution of this problem. Neither a person with ordinary skills nor the best specialist in life sciences is not able to solve it within current scientific paradigm.

Essential elements are not monoisotopic. Therefore, even the lightest isotope of any essential element has an excess of neutrons over protons and therefore an imbalance between the fields \vec{Π}ₑ and \vec{Π}ₘ.

Moreover, even if this imbalance is eliminated, the field \vec{Π}ₘ will remain uncompensated for the second, third and so on isotopes.

### Methods and Pharmaceutical Compositions

This disclosure provides a method to restore homeostasis in a subject in need thereof by establishing or rebuilding a homeostatic balance between electric and magnetic components of differential and integral characteristics of electromagnetic field in cells of said subject, comprising administering to said subject a therapeutically effective amount of a pharmaceutical composition comprising various essential chemical elements and additive chemical elements specific to each essential chemical element. In some embodiments, the additive chemical elements specific to each essential chemical element is administered to the subject in specific quantities.

This disclosure also provides a method of treating or preventing a degenerative disease in a subject in need thereof by establishing or rebuilding a homeostatic balance between electric and magnetic components of differential and integral characteristics of electromagnetic field in cells of said subject, comprising administering to said subject a therapeutically effective amount of a pharmaceutical composition comprising various essential chemical elements and additive elements specific to each essential element. In some embodiments, the additive chemical elements specific to each essential chemical element is administered to the subject in specific quantities.

This disclosure also provides a method of achieving anti-aging effect in a subject in need thereof comprising establishing or rebuilding a homeostatic balance between electric and magnetic components of differential and integral characteristics of electromagnetic field in cells of said subject, by administering to said subject a therapeutically effective amount of a pharmaceutical composition comprising various essential chemical elements and additive elements specific to each essential element. In some embodiments, the additive chemical elements specific to each essential chemical element is administered to the subject in specific quantities.

In another aspect, this disclosure provides a pharmaceutical composition comprising therapeutically effective amounts of various essential chemical elements and additive chemical elements specific to each essential chemical element in specific quantities for restoring homeostasis in a subject in need thereof, by establishing or rebuilding a homeostatic balance between electric and magnetic components of differential and integral characteristics of electromagnetic field in cells of said subject.

In another aspect, this disclosure provides a system for restoring homeostasis in a subject in need thereof, comprising a pharmaceutical composition comprising therapeutically effective amounts of various essential chemical elements and additive chemical elements specific to each essential chemical element in specific quantities to be administered to said subject, after administering to said subject said composition, said system establishes or rebuilds a homeostatic balance between electric and magnetic components of differential and integral characteristics of electromagnetic field in cells of said subject.

The terms “essential element” and “essential chemical element” are used interchangeably herein. The terms “additive chemical element” and “additive element” are used interchangeably herein.

In some embodiments, each additive chemical element specific to each essential chemical element is chosen as a pair for each isotope of said additive chemical element such that number of protons in the additive chemical element is equal to number of neutrons in the corresponding isotope of the essential chemical element.

In some embodiments, the essential chemical elements and additive chemical elements have natural isotope abundance.

In some embodiments, each essential chemical element contains at least 80% of its lightest isotope and the additive chemical elements have natural isotope abundance.

In some embodiments, the essential chemical elements have natural isotope abundance and at least one additive chemical element contains at least 80% of its lightest isotope.

In some embodiments, the essential chemical elements contain at least 80% of its lightest isotope and each of the additive chemical elements contains at least 80% of its lightest isotope.

In some embodiments, a therapeutic dose of each additive chemical elements is chosen by calculating as a product between ratio of the daily consumption dose of the additive chemical element that compensates for the magnetic component of the lightest isotope of one of the essential chemical elements to the abundance of said isotope in nature and the abundance of each isotope of said essential chemical element.

In some embodiments, the pharmaceutical composition comprises the essential chemical element Fe and additive chemical elements Ni, Zn, Ga and Ge, and a therapeutic dose equal to or multiple of a sum of: 20 mg Fe+0.15 mg Ni+2.36 mg Zn+50 mcg Ga+7.2 mcg Ge.

In some embodiments, the pharmaceutical composition comprises the essential chemical element Ni and additive chemical elements Zn, Ge, As and Se, and a therapeutic dose equal to or multiple of a sum of: 0.15 mg Ni+20 mg Zn+7.7 mg Ge+335 mcg As+1.07 mg Se.

In some embodiments, the pharmaceutical composition comprises the essential chemical element Zn and additive chemical elements Se, Rb, Sr and Zr, and a therapeutic dose equal to or multiple of a sum of: 20 mg Zn+60 mcg Se+4.94 mcg Rb+22.84 mg Sr+0.74 mcg Zr.

In some embodiments, the pharmaceutical composition comprises the essential chemical element Mg and additive chemical elements Al and Si, and a therapeutic dose equal to or multiple of a sum of: 370 mg Mg+10 mg Al+1.39 mg Si.

In some embodiments, the pharmaceutical composition comprises the essential chemical element K and additive chemical elements Ca, Sc and Ti, and a therapeutic dose equal to or multiple of a sum of: 3500 mg K+2500 mg Ca+320 mcg Sc+169 mg Ti.

In some embodiments, the pharmaceutical composition comprises the essential chemical element Mn and additive chemical elements Zn, Se, Rb, Sr, Zr and a therapeutic dose equal to or multiple of a sum of: 2.3 mg Mn+20 mg Zn+60 mcg Se+4.94 mcg Rb+22.84 mg Sr+0.74 mcg Zr.

In some embodiments, the pharmaceutical composition comprises the essential chemical element Fe enriched to 90% isotope Fe-54 and additive chemical elements Ni, Zn, Ga and Ge, and a therapeutic dose equal to or multiple of a sum of: 20 mg Fe-54+2.31 mg Ni+0.23 mg Zn+26 mg Ga+2.6 mg Ge.

In some embodiments, the pharmaceutical composition comprises the essential chemical element Zn enriched to 99, 31% isotope Zn-64 and additive chemical elements Se, Rb, Sr and Zr, and a therapeutic dose equal to or multiple of a sum of: 20 mg Zn-64+122.6 mcg Se+0.06 mcg Rb+0.26 mg Sr+0.01 mcg Zr.

In some embodiments, the pharmaceutical composition comprises the essential chemical element Se enriched to 99.30% isotope Se-74 and additional elements Sr and Mo and therapeutic dose equal to or multiple of a sum of: 60 mcg Se-74+55.3 mg Zr+5.14 mcg Mo.

In some embodiments, the pharmaceutical composition comprises the essential chemical element Mo enriched to 92.0% isotope Mo-92 and additional element Sn and a therapeutic dose equal to or multiple of a sum of: 100 mcg Mo-92+20.27 mcg Sn.

In some embodiments, the pharmaceutical composition further comprises a carrier. In further embodiments, the carrier is at least one oxide, sulfate, citrate, gluconate, or chelate.

In some embodiments, the pharmaceutical composition is administered by injection. In some embodiments, the pharmaceutical composition is administered orally.

The essential chemical elements include Mo, Se, Zn, Fe, Mn, Ni, and Mg.

### Formulating and Administering Compositions

The disclosed composition may be administered to a subject in need thereof by any suitable mode of administration, any suitable frequency, and at any suitable, effective dosage.

The composition for use in a disclosed method may be in any suitable form and may be formulated for any suitable means of delivery. In some embodiments, the composition for use in a disclosed method is provided in a form suitable for oral administration, such as a tablet, pill, lozenge, capsule, liquid suspension, liquid solution, or any other conventional oral dosage form. The oral dosage forms may provide immediate release, delayed release, sustained release, or enteric release, and, if appropriate, comprise one or more coating. In some embodiments, the disclosed composition is provided in a form suitable for injection, such as subcutaneous, intramuscular, intravenous, intraperitoneal, or any other route of injection. In some embodiments, compositions for injection are provided in sterile and/or non-pyrogenic form and may contain preservatives and/or other suitable excipients, such as sucrose, sodium phosphate dibasic heptahydrate or other suitable buffer, a pH-adjusting agent such as hydrochloric acid or sodium hydroxide, and polysorbate 80 or other suitable detergent.

When provided in solution form, in some embodiments, the composition for use in a disclosed method is provided in a glass or plastic bottle, vial or ampoule, any of which may be suitable for either single or multiple use. The bottle, vial or ampoule containing the disclosed composition may be provided in kit form together with one or more needles of suitable gauge and/or one or more syringes, all of which preferably are sterile. Thus, in certain embodiments, a kit is provided comprising a liquid solution as described above, which is packaged in a suitable glass or plastic bottle, vial or ampoule and may further comprising one or more needles and/or one or more syringes. The kit may further comprise instruction for use.

The composition for use in a disclosed method can be produced by methods employed in accordance with general practice in the pharmaceutical industry, such as, for example, the methods illustrated in Remington: The Science and Practice of Pharmacy (Pharmaceutical Press; 21st revised ed. (2011) (hereinafter “Remington”).

In some embodiments, the composition for use in a disclosed method comprise at least one pharmaceutically acceptable vehicle or excipient. These include, for example, diluents, carriers, excipients, fillers, disintegrants, solubilizing agents, dispersing agents, preservatives, wetting agents, preservatives, stabilizers, buffering agents (e.g. phosphate, citrate, acetate, tartrate), suspending agents, emulsifiers, and penetration enhancing agents such as DMSO, as appropriate. The composition can also comprise suitable auxiliary substances, for example, solubilizing agents, dispersing agents, suspending agents and emulsifiers.

In certain embodiments, the composition further comprises suitable diluents, glidants, lubricants, acidulants, stabilizers, fillers, binders, plasticizers or release aids and other pharmaceutically acceptable excipients.

A complete description of pharmaceutically acceptable excipients can be found, for example, in Remington's Pharmaceutical Sciences (Mack Pub., Co., N.J. 1991) or other standard pharmaceutical science texts, such as the Handbook of Pharmaceutical Excipients (Shesky et al. eds., 8th ed. 2017).

In some embodiments, the composition for use in a disclosed method can be administered intragastrically, orally, intravenously, intraperitoneally or intramuscularly, but other routes of administration are also possible.

Water may be used as a carrier and diluent in the composition. The use of other pharmaceutically acceptable solvents and diluents in addition to or instead of water is also acceptable. In certain embodiments, deuterium-depleted water is used as a diluent.

Large macromolecules that are slowly metabolized, such as proteins, polysaccharides, polylactic acids, polyglycolic acids, polymeric amino acids, copolymers of amino acids, can also be used as carrier compounds for the composition. Pharmaceutically acceptable carriers in therapeutic compositions may additionally contain liquids, such as water, saline, glycerol or ethanol. Moreover, the said compositions may further comprise excipients, such as wetting agents or emulsifiers, buffering substances, and the like. Such excipients include, among others, diluents and carriers conventional in the art, and/or substances that promote penetration of the active compound into the cell, for example, DMSO, as well as preservatives and stabilizers.

The composition for use in a disclosed method may be presented in various dosage forms depending on the object of application; in particular, it may be formulated as a solution for injections.

The composition for use in a disclosed method may be administered systemically. Suitable routes of administration include, for example, oral or parenteral administration, such as intravenous, intraperitoneal, intragastric as well as via drinking water. However, depending on a dosage form, the disclosed composition may be administered by other routes.

The disclosed composition can be co-administered with another appropriate agent or therapy.

### EXAMPLES

The following examples are for purposes of illustration only and are not to be construed as limiting the scope of the invention in any manner.

### Example 1. The Use of Natural Iron as a Component of a Therapeutic Composition

Iron has four naturally occurring stable isotopes: ⁵⁴Fe (5.84%), ⁵⁶Fe (91.76%), ⁵⁷Fe (2.12%), and ⁵⁸Fe (0.28%).

Isotope Fe-54 has an atomic weight of 54, with 26 protons, 26 electrons and 28 neutrons. Earlier it was demonstrated that in a field of \vec{Π}m (magnetic) 28 neutrons represent the “magnetic” component of an atom consisting of 28 magnetic analogs of protons and 28 magnetic analogs of electrons (positive and negative magnetic charges). Then, 26 protons and 26 electrons refer to the electric component of the atom in a field of \vec{Π}e (electric). As a result, it is concluded:

An isotope of Fe-54 can be thought of as a combination of two components of “electric” iron and “magnetic” nickel. It is this magnetic nickel that upsets the balance of fields \vec{Π} e and \vec{Π} m. This view allows to immediately find a solution to the problem: “magnetic” nickel must be compensated by the addition of “electric” nickel which has \vec{Π}e component with 28 protons and 28 electrons.

Then, isotope of Fe-56 can be thought of as a combination of two components of “electric” iron and “magnetic” zinc, isotope of Fe-57 as “electric” iron and “magnetic” Ga, and isotope Fe-58 as “electric” iron and “magnetic” germanium. “Magnetic” zinc, gallium and germanium must be compensated by the addition of “electric” zinc, gallium and germanium which have \vec{Π} e components with 30 protons and 30 electrons, 31 protons and 31 electrons, and 32 protons and 32 electrons respectively.

This means that for therapeutic purposes, natural iron (the essential chemical element) must be used in combination with chemical elements nickel, zinc, gallium and germanium (the additive chemical elements).

The effectiveness of the therapeutic combination is defined by the correct choice of doses of the constituent components. It is impossible to use known daily consumption doses, since the compensating components can play the same role in the case of other essential elements. Therefore, as a benchmark, one takes the ratio of the daily consumption dose of the element that compensates for the magnetic component of the lightest isotope (in this case it is nickel) to the abundance of this isotope in natural element. Then, the concentration of other components can be calculated as the product between this ratio and ratios of other isotopes the magnetic component of which must be compensated.

Consider daily consumption dose of natural iron as equal to 20 mg. The natural abundance of Fe-54 is 5.84%. To compensate for the magnetic component of Fe-54 in natural iron, one needs 0.15 mg of nickel. Then, the benchmark/reference ratio is 0.15 mg/5, 84%. To find the amount of Zn to compensate for the magnetic component of Fe-56 one needs to multiply this ratio on the abundance of isotope Fe-56, which is 91, 76%. One gets 0.15/5.84×91.76=2.36 mg of zinc, which is about 10 times less than daily consumption dose of zinc. The same procedure for gallium and germanium provides values of 50 mcg and 7.2 mcg correspondently which are also very different from the known daily consumption doses.
Fe 20 mg dose of Fe
Ni 0.15 mg to compensate “magnetic” Ni in Fe-54
Zn 2.36 mg to compensate “magnetic” Zn in Fe-56
Ga 50 mg to compensate “magnetic” Ga in Fe-57
Ge 7.2 mg to compensate “magnetic” Ge in Fe-58

This composition will provide a much better balance between \vec{Π}e and \vec{Π}m fields and healthy homeostasis in live cells of the human organism.

### Example 2. The Use of Enriched Lightest Iron Isotope Fe-54 as the Component of a Therapeutic Compound

Iron Fe-54 isotope enriched to 90% has four stable isotopes: ⁵⁴Fe (90.00%), ⁵⁶Fe (8.90%), ⁵⁷Fe (1.00%), and ⁵⁸Fe (0.10%).

Isotope Fe-54 has an atomic weight of 54, with 26 protons, 26 electrons and 28 neutrons. Earlier it was demonstrated that in a field of \vec{Π}m 28 neutrons represent the magnetic component of an atom consisting of 28 magnetic analogs of protons and 28 magnetic analogs of electrons (positive and negative magnetic charges). Then, 26 protons and 26 electrons refer to the electric component of the atom in a field of \vec{Π}e.

An isotope of Fe-54 can be thought of as a combination of two components of “electric” iron and “magnetic” nickel. It is this magnetic nickel that upsets the balance of fields \vec{Π}e and \vec{Π}m. This view allows to immediately find a solution to the problem: “magnetic” nickel must be compensated by the addition of “electric” nickel which has \vec{Π}e component with 28 protons and 28 electrons.

Then, isotope of Fe-56 can be thought of as a combination of two components of “electric” iron and “magnetic” zinc, isotope of Fe-57 as “electric” iron and “magnetic” Ga, and isotope Fe-58 as “electric” iron and “magnetic” germanium. “Magnetic” zinc, gallium and germanium must be compensated by the addition of “electric” zinc, gallium and germanium which have \vec{Π}e components with 30 protons and 30 electrons, 31 protons and 31 electrons, and 32 protons and 32 electrons respectively.

This means that for therapeutic purposes, iron isotope Fe-54 must be used in combination with chemical elements nickel, zinc, and germanium.

Here, as a benchmark, one takes the ratio of the daily consumption dose of the element that compensate for the magnetic component of the lightest isotope (in this case it is nickel) to the abundance of this isotope in natural element. Then, the concentration of other components can be calculated as the product between this ratio and ratios of other isotopes the magnetic component of which must be compensated.

Consider daily consumption dose of natural iron as equal to 20 mg. The natural abundance of Fe-54 is 5.84%. To compensate for the magnetic component of Fe-54 in natural iron one needs 0.15 mg of nickel. Then, the benchmark/reference ratio is 0.15 mg/5.84%. To find the amount of nickel to compensate for the magnetic component of Fe-54 we need to multiply this ratio on the abundance of Fe-54 enriched to 90%: 0.15 mg/5.84%×8.9%=2.31 mg. To find the amount of Zn to compensate for the magnetic component of Fe-56 we need to multiply this ratio on the abundance of isotope Fe-56 which is 8.90%. We get 0.15/5.84×8.90=0.23 mg of zinc which is about 100 times less than daily consumption dose of zinc. The same procedure for gallium and germanium provides values of 26 mcg and 2.6 mcg correspondently which are also very different from the known daily consumption doses.
Fe 20 mg dose of Fe-54
Ni 2.31 mg to compensate “magnetic” Ni in Fe-54
Zn 0.23 mg to compensate “magnetic” Zn in Fe-56
Ga 26 mg to compensate “magnetic” Ga in Fe-57
Ge 2.6 mg to compensate “magnetic” Ge in Fe-58

This composition will provide a much better balance between \vec{Π}e and \vec{Π}m fields and healthy homeostasis in live cells of the human organism.

The rational above helps to understand why chemical elements which have no natural function in biology (nickel, gallium, and germanium) may have therapeutic activity and to explain why gallium, a group IIIA metal shares chemical properties with iron. Bernstein, L. R. et al., (2000). Metal-Based Drugs. 7 (1): 33-47.

“The body needs nickel, but in very small amounts. Nickel is a common trace element in multiple vitamins. Nickel is used for increasing iron absorption, preventing iron-poor blood (anemia), and treating weak bones (osteoporosis)”. https://www.emedicinehealth.com/nickel/vitamins-supplements.htm

“Although gallium has no known physiologic function in the human body, certain of its characteristics enable it to interact with cellular processes and biologically important proteins, especially those of iron metabolism”. Christopher R. Chitambar. Int J Environ Res Public Health. 2010 May; 7 (5): 2337-2361. Published online 2010 May 10.

It is also clear the reason for the contradiction of data on the therapeutic action of individual essential elements or their combination. Outside of the model being used, it is impossible to correctly choose a therapeutic element or combination of elements and to calculate proper doses of these elements to balance \vec{Π}e and \vec{Π}m fields inside live cells.

### Example 3. The Use of Natural Nickel as the Component of a Therapeutic Compound

Naturally occurring nickel (28Ni) is composed of five stable isotopes; 58Ni, 60Ni, 61Ni, 62Ni and 64Ni, with 58Ni the most abundant (68.077% natural abundance).

Isotope Ni-58 has an atomic weight of 58, 28 protons, 28 electrons and 30 neutrons. Earlier it was demonstrated that in a field of \vec{Π}m 30 neutrons represent the “magnetic” component of an atom consisting of 30 magnetic analogs of protons and 30 magnetic analogs of electrons (positive and negative magnetic charges). Then, 28 protons and 28 electrons refer to the electric component of the atom in a field of \vec{Π}e.

An isotope of Ni-58 can be thought of as a combination of two components of “electric” nickel and “magnetic” zinc. It is this magnetic zinc that upsets the balance of fields \vec{Π}e and \vec{Π}m. This view allows to immediately find a solution to the problem: “magnetic” zinc must be compensated by the addition of “electric” zinc which has \vec{Π}e component with 30 protons and 30 electrons.

Then, isotope of Ni-60 can be thought of as a combination of two components of “electric” nickel and “magnetic” germanium, isotope of Ni-61 as “electric” nickel and “magnetic” As, isotope Ni-62 as “electric” nickel and “magnetic” selenium, isotope Ni-64 as electric nickel and magnetic krypton. “Magnetic” germanium, arsenic and selenium must be compensated by the addition of “electric” germanium, arsenic and selenium which have \vec{Π}e components with 32 protons and 32 electrons, 33 protons and 33 electrons, and 34 protons and 34 electrons respectively. One must ignore krypton due to lack of the possibility to use “electric” krypton. At the same time the abundance of Ni-64 is very small (0.926%) and, besides it is probably already taken care of by nature and leaving this isotope magnetic component uncompensated is acceptable.

This means that for therapeutic purposes, natural nickel must be used in combination with chemical elements zinc, germanium, arsenic, and selenium.

Here, as a benchmark, one takes the ratio of the daily consumption dose of the element that compensate for the magnetic component of the lightest isotope (in this case it is zinc) to the abundance of this isotope in natural element. Then, the concentration of other components can be calculated as the product between this ratio and ratios of other isotopes the magnetic component of which must be compensated.

Consider daily consumption dose of natural nickel as equal to 0.15 mg. The natural abundance of Ni-58 is 68.077%. To compensate for the magnetic component of Ni-58 in natural nickel one needs 20 mg of zinc. Then, the benchmark/reference ratio is 20 mg/68.077%. To find the amount of Ge to compensate for the magnetic component of Ni-60 one needs to multiply this ratio on the abundance of isotope Ni-60 which is 26.223%. One gets 20 mg/68.077%×26.223%=7.7 mg of Ge, which is about 3 times more than daily consumption dose of germanium. The same procedure for arsenic and selenium provides values of 0,335 mg and 1.07 mg correspondently.
Ni 0.15 mg dose of Ni-58
Zn 20 mg to compensate “magnetic” Zn in Ni-58
Ge 7.7 mg to compensate “magnetic” Ge in Ni-60
As 335 mcg to compensate “magnetic” As in Ni-61
Se 1.07 mcg to compensate “magnetic” Se in Ni-62

This composition will provide a much better balance between \vec{Π} e and \vec{Π} m fields and healthy homeostasis in live cells of the human organism.

The need for arsenic may seem dubious. Therefore, it makes sense to present here considerations presented in the article Phillip Hunter. EMBO Rep., 2008 January; 9 (1): 15-18: “It seems that arsenic has a role in the metabolism of the amino acid methionine and in gene silencing. Other work suggests that it has a positive interaction with the more important micronutrient selenium. In fact, if arsenic is essential for humans, its recommended daily intake would be little different from selenium, which is so important that evolution incorporated it into the rare amino acid selenocysteine—the crucial component of the antioxidizing selenoproteins that help to repair other proteins from oxidative damage”.

### Example 4. The Use of Natural Zinc as the Component of a Therapeutic Compound

Naturally occurring zinc (30Zn) is composed of the 5 stable isotopes 64Zn, 66Zn, 67Zn, 68Zn, and 70Zn with 64Zn being the most abundant (48.6% natural abundance).

Isotope Zn-64 has an atomic weight of 64, 30 protons, 30 electrons and 34 neutrons. Earlier it was demonstrated that in a field of \vec{Π}m 34 neutrons represent the “magnetic” component of an atom consisting of 34 magnetic analogs of protons and 34 magnetic analogs of electrons (positive and negative magnetic charges). Then, 30 protons and 30 electrons refer to the electric component of the atom in a field of \vec{Π}e.

An isotope of Zn-64 can be thought of as a combination of two components of “electric” zinc and “magnetic” selenium. It is this magnetic selenium that upsets the balance of fields \vec{Π}e and \vec{Π}m. This view allows to immediately find a solution to the problem: “magnetic” selenium must be compensated by the addition of “electric” selenium which has \vec{Π}e component with 34 protons and 34 electrons.

Then, isotope of Zn-66 can be thought of as a combination of two components of “electric” zinc and “magnetic” krypton, isotope of Zn-67 as “electric” zinc and “magnetic” Rb, isotope Zn-68 as “electric” zinc and “magnetic” strontium, isotope Zn-70 as electric zinc and magnetic zirconium. “Magnetic” selenium, krypton, rubidium, strontium, and zirconium must be compensated by the addition of “electric” selenium, krypton, rubidium, strontium, and zirconium which have e components with 34 protons and 34 electrons, 36 protons and 36 electrons, and 37 protons and 37 electrons, 38 protons and 38 electrons, 40 protons and 40 electrons respectively. One must ignore krypton due to lack of the possibility to use “electric” krypton. One assumes that it is probably already taken care of by nature and leaving this isotope magnetic component uncompensated is acceptable.

This means that for therapeutic purposes, natural zinc must be used in combination with chemical elements selenium, rubidium, strontium, and zirconium.

Here, as a benchmark, one takes the ratio of the daily consumption dose of the element that compensate for the magnetic component of the lightest isotope (in this case it is selenium) to the abundance of this isotope in natural element. Then, the concentration of other components can be calculated as the product between this ratio and ratios of other isotopes the magnetic component of which must be compensated.

Consider daily consumption dose of natural zinc as equal to 20 mg. The natural abundance of Zn-64 is 48.6%. To compensate for the magnetic component of Zn-64 in natural zinc one needs 60 mcg of selenium. Then, the benchmark/reference ratio is 60 mcg/48, 6%. To find the amount of Rb to compensate for the magnetic component of Zn-67 one needs to multiply this ratio on the abundance of isotope Zn-67 which is 4.0%. One gets 60 mcg/48, 6%×4.0%=4.94 mcg of Rb, which is much less than the daily consumption dose of rubidium. The same procedure for strontium and zirconium provides values of 22.84 mcg and 0.74 mcg correspondently.
Zn 20 mg dose of Zn natural
Se 60 mcg to compensate “magnetic” Se in Zn-64
Rb 4.94 mcg to compensate “magnetic” Rb in Zn-67
Sr 22.84 mg to compensate “magnetic” Sr in Zn-68
Zr 0.74 mg to compensate “magnetic” Zr in Zn-70

This composition will provide a much better balance between \vec{Π}e and \vec{Π}m fields and healthy homeostasis in live cells of the human organism.

### Example 5. The Use of Highly Enriched Lightest Zinc Isotope Zn-64 as a Component of a Therapeutic Compound

Zn-64 isotope enriched to 99, 31% has five stable isotopes: 64Zn (99.31%), 66Zn (0.42%), 67Zn (0.05%), and 68Zn (0.21%) and 70Zn (0.01%).

Isotope Zn-64 has an atomic weight of 64, 30 protons, 30 electrons and 34 neutrons. Earlier it was demonstrated that in a field of \vec{Π}m 34 neutrons represent the “magnetic” component of an atom consisting of 34 magnetic analogs of protons and 34 magnetic analogs of electrons (positive and negative magnetic charges). Then, 30 protons and 30 electrons refer to the electric component of the atom in a field of Me.

An isotope of Zn-64 can be thought of as a combination of two components of “electric” zinc and “magnetic” selenium. It is this magnetic selenium that upsets the balance of fields \vec{Π}e and \vec{Π}m. This view allows to immediately find a solution to the problem: “magnetic” selenium must be compensated by the addition of “electric” selenium which has \vec{Π}e component with 34 protons and 34 electrons.

Then, isotope of Zn-66 can be thought of as a combination of two components of “electric” zinc and “magnetic” krypton, isotope of Zn-67 as “electric” zinc and “magnetic” Rb, isotope Zn-68 as “electric” zinc and “magnetic” strontium, isotope Zn-70 as electric zinc and magnetic zirconium. “Magnetic” selenium, krypton, rubidium, strontium, and zirconium must be compensated by the addition of “electric” selenium, krypton, rubidium, strontium, and zirconium which have e components with 34 protons and 34 electrons, 36 protons and 36 electrons, and 37 protons and 37 electrons, 38 protons and 38 electrons, 40 protons and 40 electrons respectively. One must ignore krypton due to lack of the possibility to use “electric” krypton. One assumes that it is probably already taken care of by nature and leaving this isotope magnetic component uncompensated is acceptable.

This means that for therapeutic purposes, enriched zinc-64 must be used in combination with chemical elements selenium, rubidium, strontium, and zirconium.

Here, as a benchmark, one takes the ratio of the daily consumption dose of the element that compensate for the magnetic component of the lightest isotope (in this case it is selenium) to the abundance of this isotope in natural element. Then, the concentration of other components can be calculated as the product between this ratio and ratios of other isotopes the magnetic component of which must be compensated.

Consider daily consumption dose of natural zinc as equal to 20 mg. The natural abundance of Zn-64 is 48, 6%. To compensate for the magnetic component of Zn-64 in natural zinc one needs 60 mcg of selenium. Then, the benchmark/reference ratio is 60 mcg/48, 6%. To find the amount of selenium to compensate for the magnetic component of Zn-64 in the zinc enriched to 99, 31% of Zn-64 one needs to multiply this ratio on 99, 31%. To find the amount of Rb to compensate for the magnetic component of Zn-67 one needs to multiply this ratio on the abundance of isotope Zn-67, which is 0.05%. The same procedure for strontium and zirconium provides values of 0.26 mcg and 0.01 mcg correspondently.
Zn 20 mg dose of Zn-64
Se 122.60 mcg to compensate “magnetic” Se in Zn-64
Rb 0.06 mcg to compensate “magnetic” Rb in Zn-67
Sr 0.26 mcg to compensate “magnetic” Sr in Zn-68
Zr 0.01 mcg to compensate “magnetic” Zr in Zn-70

This composition will provide a much better balance between \vec{Π} e and \vec{Π} m fields and healthy homeostasis in live cells of the human organism.

### Example 6. The Use of Selenium as the Component of a Therapeutic Compound

Selenium (34Se) has five stable natural isotopes that occur in significant quantities: 74Se (0.86%), 76Se (9.23%), 77Se (7.60%), 78Se (23, 69%), and 80Se (49, 80%).

Isotope Se-74 has an atomic weight of 74, 34 protons, 34 electrons and 40 neutrons. Earlier it was demonstrated that in a field of \vec{Π}m 40 neutrons represent the “magnetic” component of an atom consisting of 40 magnetic analogs of protons and 40 magnetic analogs of electrons (positive and negative magnetic charges). Then, 34 protons and 34 electrons refer to the electric component of the atom in a field of \vec{Π}e.

An isotope of Se-74 can be thought of as a combination of two components of “electric” selenium and “magnetic” zirconium. It is this magnetic zirconium that upsets the balance of fields \vec{Π}e and \vec{Π}m. This view allows to immediately find a solution to the problem: “magnetic” zirconium must be compensated by the addition of “electric” zirconium which has \vec{Π}e component with 40 protons and 40 electrons.

Then, isotope of Se-76 can be thought of as a combination of two components of “electric” selenium and “magnetic” molybdenum, isotope of Se-77 as “electric” selenium and “magnetic” technetium, isotope Se-78 as “electric” selenium and “magnetic” ruthenium, isotope Se-80 as electric selenium and magnetic palladium. “Magnetic” zirconium, molybdenum, technetium, ruthenium, and palladium must be compensated by the addition of “electric” zirconium, molybdenum, technetium, ruthenium, and palladium which have e components with 40 protons and 40 electrons, 42 protons and 42 electrons, and 43 protons and 43 electrons, 44 protons and 44 electrons, 46 protons and 46 electrons respectively.

This means that for therapeutic purposes, selenium must be used in combination with chemical elements zirconium, molybdenum, technetium, ruthenium, and palladium.

Technetium (43Tc) is the first of the two elements lighter than bismuth that have no stable isotopes and cannot be used. Ruthenium considered to be highly toxic. Palladium is regarded as of low toxicity but has no biological role. Therefore, it is impossible to compensate “magnetic components” of Se-77, Se-78, and Se-80. These isotopes of selenium have high isotope ratios/percentage in natural abundance and pose a threat to the cellular homeostasis. Hence it follows that it is impossible to use natural selenium as a therapeutic agent or food supplement. At the same time selenium is essential for the life of a living cells. Therefore, a logical solution is to use the highly enriched isotope Se-74 along with zirconium and molybdenum to compensate magnetic component \vec{Π}m of isotopes Se-74 and Se-76.

Consider the use of enriched lightest isotope (minimum excess of neutrons over protons) of Se-74 with the isotope ratios Se-74 (99.3%), Se-76 (0.2%), Se-77 (0.1%), Se-78 (0.1%) and Se-80 (0.3%).

Here, as a benchmark, one takes the ratio of the daily consumption dose of the element that compensate for the magnetic component of the lightest isotope (in this case it is selenium) to the abundance of this isotope in natural element. Then, the concentration of other components can be calculated as the product between this ratio and ratios of other isotopes the magnetic component of which must be compensated.

Consider daily consumption dose of natural selenium as equal to 60 mcg. The natural abundance of Se-74 is 0.86%. To compensate for the magnetic component of Se-74 in natural selenium one needs 479 mcg of zirconium. Then, the benchmark/reference ratio is 479 mcg/0.86%. To find the amount of zirconium to compensate for the magnetic component of Se-74 in the selenium enriched to 99.3% of Se-74 one needs to multiply this ratio on 99.3%. To find the amount of Mo to compensate for the magnetic component of Se-76 one needs to multiply this ratio on the abundance of isotope Se-76, which is 9.23%.
Se 60 mcg dose of Se-74 (99.3% enrichment)
Zr 55.3 mcg to compensate “magnetic” Se in Se-74
Mo 5.14 mcg to compensate “magnetic” Se in Se-76

This composition will provide a much better balance between \vec{Π}e and \vec{Π}m fields and healthy homeostasis in live cells of the human organism.

The molybdenum currently used for both intravenous injections and nutritional supplementation is a naturally occurring selenium. To maintain stability, live cells will try to compensate for the heavy selenium isotopes and in particular the isotope Se-78 which will inevitably lead to the accumulation of the highly toxic ruthenium.

Compensatory doses for zirconium and molybdenum are higher than corresponding daily consumption doses. The results above explain the nature of accumulation of Zr, Mo and Ru in live cells but also indicate that the best we can do is to use selenium-74 in combination with just daily recommended doses of Zr and Mo.

### Example 7. The Use of Molybdenum as the Component of a Therapeutic Compound

Molybdenum (42Mo) has 33 known isotopes, ranging in atomic mass from 83 to 115, as well as four metastable nuclear isomers. Seven isotopes occur naturally, with atomic masses of 92, 94, 95, 96, 97, 98, and 100. All unstable isotopes of molybdenum decay into isotopes of zirconium, niobium, technetium, and ruthenium. Lide, David R., ed. (2006). CRC Handbook of Chemistry and Physics (87th ed.). Boca Raton, Florida: CRC Press. Section 11.

It is quite interesting to note that unstable isotopes of molybdenum decay into four isotopes, three of which are compensation isotopes for selenium and molybdenum itself is a compensation element for the isotope Se-76. Indirectly, it means that the vector field \vec{Π}m is primary in relation to the \vec{Π}e field and also that a balance between \vec{Π}e and \vec{Π}m components is essential for the atom's stability.

Isotope Mo-92 has an atomic weight of 92, 42 protons, 42 electrons and 50 neutrons. Earlier it was demonstrated that in a field of \vec{Π}m 50 neutrons represent the “magnetic” component of an atom consisting of 50 magnetic analogs of protons and 50 magnetic analogs of electrons (positive and negative magnetic charges). Then, 42 protons and 42 electrons refer to the electric component of the atom in a field of \vec{Π}e.

An isotope of Mo-92 can be thought of as a combination of two components of “electric” molybdenum and “magnetic” tin.

It is this “magnetic” tin that upsets the balance of fields \vec{Π}e and \vec{Π}m. This view allows to immediately find a solution to the problem: “magnetic” tin must be compensated by adding the “electric” tin, which has \vec{Π} e component with 50 protons and 50 electrons.

Then, isotope of Mo-94 can be thought of as a combination of two components of “electric” molybdenum and “magnetic” Te, isotope of Mo-95 as “electric” molybdenum and “magnetic” iodine, isotope Mo-96 as “electric” molybdenum and “magnetic” Xe, isotope Mo-97 as electric molybdenum and magnetic Cs, isotope of Mo-98 as “electric” molybdenum and “magnetic” barium, isotope of Mo-100 as “electric” molybdenum and “magnetic” cerium. Analysis of biological role and toxicity of compensatory elements leads to a conclusion that the use of natural molybdenum does not make sense and causes the accumulation of harmful elements. At the same time molybdenum is essential for the life of a living cells. Therefore, a logical solution is to use the highly enriched isotope Mo-92 along with Sn to compensate magnetic component m of isotope Se-92.

Consider the use of enriched lightest isotope (minimum excess of neutrons over protons) of Mo-92 with the isotope ratio for Mo-92 equal to 99.0%.

Here, as a benchmark, one takes the ratio of the daily consumption dose of the element that compensate for the magnetic component of the lightest isotope (in this case it is tin) to the abundance of this isotope in natural element. Then, the concentration of other components can be calculated as the product between this ratio and ratios of other isotopes the magnetic component of which must be compensated.

Consider daily consumption dose of natural molybdenum as equal to 100 mcg. The natural abundance of Mo-92 is 14, 65%. Daily consumption dose of tin is 4 mg. To compensate for the magnetic component of Mo-92 enriched to 99% we need 4 mg/14, 65%×99%=27 mg of tin.
Mo 100 mcg dose of Mo-92 (99.0% enrichment)
Sn 20.27 mg to compensate “magnetic” Sn in Mo-92

This composition will provide a much better balance between \vec{Π}e and \vec{Π}m fields and healthy homeostasis in live cells of the human organism.

The molybdenum currently used for both intravenous injections and nutritional supplementation is a naturally occurring molybdenum. To maintain stability, live cells will try to compensate for the heavy molybdenum isotopes and in particular the isotope Mo-94, Mo-98, Mo-100 which will inevitably lead to the accumulation of the mildly to moderately toxic Te, Ba, and Ce.

### Example 8. The Use of Magnesium as the Component of a Therapeutic Compound

Magnesium (12 Mg) naturally occurs in three stable isotopes, 24 Mg (79.0%), 25 Mg (10.0%), and 26 Mg (11%).

Isotope Mg-24 has an atomic weight of 24, 12 protons, 12 electrons and 12 neutrons. Earlier it was demonstrated that in a field of \vec{Π}m 12 neutrons represent the “magnetic” component of an atom consisting of 12 magnetic analogs of protons and 12 magnetic analogs of electrons (positive and negative magnetic charges). Then, 12 protons and 12 electrons refer to the electric component of the atom in a field of \vec{Π}e. As a result, one concludes:

An isotope of Mg-24 can be thought of as a combination of two balanced components of “electric” magnesium and “magnetic” magnesium which form a balance of fields e and m.

Then, isotope of Mg-25 can be thought of as a combination of two components of “electric” magnesium and “magnetic” aluminum, isotope of Mg-26 as “electric” magnesium and “magnetic” Si. “Magnetic” aluminum and silicon must be compensated by the addition of “electric” aluminum and silicon which have \vec{Π}e components with 13 protons and 13 electrons, and 14 protons and 14 electrons respectively.

This means that for therapeutic purposes, natural magnesium must be used in combination with chemical elements aluminum and silicon.

Here, as a benchmark, one takes the ratio of the daily consumption dose of the element that compensate for the magnetic component of the lightest isotope (in this case it is aluminum) to the abundance of this isotope in natural element. Then, the concentration of other components can be calculated as the product between this ratio and ratios of other isotopes the magnetic component of which must be compensated.

Consider daily consumption dose of natural magnesium as equal to 370 mg. The natural abundance of Mg-24 is 79.0%. To compensate for the magnetic component of Mg-24 in natural magnesium one needs 10 mg of aluminum. Then, the benchmark/reference ratio is 10 mg/79, 0%. To find the amount of Si to compensate for the magnetic component of Mg-26 one needs to multiply this ratio on the abundance of isotope Mg-26 which is 11%. One gets 10 mg/79%×11.0%=1.39 mg of Si which is much less than the daily consumption dose of silicon.
Mg 370 mg dose of Mg-24
Al 10 mg to compensate “magnetic” Al in Mg-25
Si 1.39 mg to compensate “magnetic” Si in Mg-26

The problem is that aluminum is a poison that acts on the nervous system and has been linked to several serious health problems. A high daily intake of aluminum can be explained by the desire of a live cell to compensate for the magnetic component m of the isotope Mg-25. Natural magnesium enters the human body through food and water. Natural abundance of the isotope Mg-25 is 10%. The only way to reduce it is to consume daily intake dose of magnesium in the form of highly enriched Mg-24. At the same time, the use of an enriched isotope Mg-24 for therapeutic purposes, as was suggested in A. A. Ivanov. Russian journal of physical chemistry B, v.2, No 6, pp. 649-652, 2007; Paul Dirac, Proc. Roy. Soc. (London) A 133, 60 (1931); Zh. Loshak, Inzh. Fiz., No. 3, 12 (2014) and H. Stumpf, Z. Naturforsch A67, 163 (2012). L. D. Landau and E. M. Lifshitz, Course of Theoretical Physics, Vol. 2: The classical theory of fields (Nauka, Moscow, 1973; Pergamon, Oxford, 1975), p.91, is not sufficient as it does not allow to neutralize the magnetic component of the isotope Mg-26.

Therefore, unlike the compositions proposed in earlier sources, it is necessary to use the following combination:

Here enriched magnesium with isotope ratios of Mg-24 (99, 2%), Mg-25 (0.20%) and Mg-26 (0.6%) are calculated.

Dose of compensatory element Si for the isotope Mg-26 must be calculated as product of the same ratio as above (10 mg/79%) and isotope ratio of Mg-26 in highly enriched (to 99.2% of Mg-24) magnesium which is 0.6%. Then, one gets 10 mg/79%×0.6%=75 mcg.
Mg-24 370 mg dose of Mg-24
Si 75 mcg to compensate “magnetic” Si in Mg-26

This composition will provide a much better balance between \vec{Π}e and \vec{Π}m fields and healthy homeostasis in live cells of the human organism.

### Example 9. The Use of Potassium as the Component of a Therapeutic Compound

Three of potassium isotopes occur naturally: the two stable forms 39K (93.3%) and 41K (6.7%), and a very long-lived radioisotope 40K (0.012%).

Isotope K-39 has an atomic weight of 39, 19 protons, 19 electrons and 20 neutrons. Earlier it was demonstrated that in a field of \vec{Π}m 20 neutrons represent the “magnetic” component of an atom consisting of 20 magnetic analogs of protons and 20 magnetic analogs of electrons (positive and negative magnetic charges). Then, 19 protons and 19 electrons refer to the electric component of the atom in a field of \vec{Π}e. As a result, one concludes:

An isotope of K-39 can be thought of as a combination of two balanced components of “electric” potassium and “magnetic” calcium. It is this magnetic calcium that upsets the balance of fields \vec{Π}e and \vec{Π}m. This view allows to find a solution to the problem: “magnetic” calcium must be compensated by the addition of “electric” calcium which has \vec{Π}e component with 20 protons and 20 electrons.

Then, isotope of K-40 can be thought of as a combination of two components of “electric” potassium and “magnetic” scandium, isotope of K-41 as “electric” potassium and “magnetic” Ti. “Magnetic” scandium and titanium must be compensated by the addition of “electric” scandium and titanium which have e components with 21 protons and 21 electrons, and 22 protons and 22 electrons respectively.

This means that for therapeutic purposes, natural potassium must be used in combination with chemical elements calcium, scandium, and titanium.

Here, as a benchmark, one takes the ratio of the daily consumption dose of the element that compensate for the magnetic component of the lightest isotope (here it is calcium) to the abundance of this isotope in natural element. Then, the concentration of other components can be calculated as the product between this ratio and ratios of other isotopes the magnetic component of which must be compensated.

Consider daily consumption dose of natural potassium as equal to 3.5 g. The natural abundance of K-39 is 93.3%. To compensate for the magnetic component of K-39 in natural potassium one needs 2.5 g of calcium. Then, the benchmark/reference ratio is 2.5 g/93.3%. To find the amount of Sc to compensate for the magnetic component of K-40 one needs to multiply this ratio on the abundance of isotope K-40, which is 0.012%. One gets 2.5 g/93.3%×0.012%=320 mcg of Sc. The same procedure for titanium provides values of 2.5 g/93.3%×6.7%=169 mg.
K 3500 mg dose of K
Ca 2500 mg to compensate “magnetic” Ca in K-39
Sc 320 mcg to compensate “magnetic” Sc in K-40
Ti 169 mcg to compensate “magnetic” Ti in K-41

“Scandium has no biological role. Only trace amounts reach the food chain, so the average person's daily intake is less than 0.1 microgram. Elemental scandium is considered non-toxic, and little animal testing of scandium compounds has been done. The half lethal dose (LD50) levels for scandium (III) chloride for rats have been determined as 4 mg/kg for intraperitoneal, and 755 mg/kg for oral administration. Kwon HoKoo, Jung Sun Park. Toxicological Evaluations of Rare Earths and Their Health Impacts to Workers: A Literature Review Safety and Health at Work, Volume 4, Issue 1, March 2013, Pages 12-26.

Titanium dioxide is possibly carcinogenic to humans, based on studies that demonstrated increased lung tumors in rats associated with titanium dioxide inhalation.

Therefore, it makes sense to use natural potassium or highly enriched K-39 in combination with calcium only. For the natural potassium it gives combination of 3500 mg of K together with 2500 mg of Ca based on daily doses.

For the enriched K-39 (99.0%) it would be 3500 mg of K-39 plus 2.5 g/99.3%×99%=2492 mg of Ca. It seems to be fine to add 320 mcg of scandium also as it is within the daily consumption dose.

This composition will provide a much better balance between \vec{Π}e and \vec{Π}m fields and healthy homeostasis in live cells of the human organism.

### Example 10. The Use of Manganese as the Component of a Therapeutic Compound

Manganese is a monoisotopic element. It has the only stable isotope Mn-55. It is an essential element and therefore, the benefits of its use are clear. However, it is not all clear whether its effectiveness as a therapeutic agent can be improved. One can suggest a combination of manganese with any other essential elements. But how can one be sure that a certain combination is correct and will lead to increased efficiency. There is the likelihood of a good random choice. It is not wise to rely on luck when it comes to therapeutic agents.

Use the approach based on the model described above. Isotope Mn-55 has an atomic weight of 55, 25 protons, 25 electrons and 30 neutrons. Earlier it was demonstrated that in a field of \vec{Π}m 30 neutrons represent the “magnetic” component of an atom consisting of 30 magnetic analogs of protons and 30 magnetic analogs of electrons (positive and negative magnetic charges). Then, 25 protons and 25 electrons refer to the electric component of the atom in a field of \vec{Π}e. An isotope of Mn-55 can be thought of as a combination of two components of “electric” manganese and “magnetic” zinc. It is this magnetic zinc that upsets the balance of fields e and m. This view allows to find a solution to the problem: “magnetic” zinc must be compensated by the addition of “electric” zinc which has \vec{Π}e component with 30 protons and 30 electrons.

This means that for therapeutic purposes, natural manganese must be used in combination with the chemical element zinc.

The therapeutic dose of zinc is equal to its daily consumption dose. Then one can significantly improve the composition using the results from Examples 4 and 5.
1. For the natural compensatory zinc additive:
Mn 2.3 mg dose of Mn-55
Zn 20 mg to compensate “magnetic” Zn in Mn-55
Se 60 mcg to compensate “magnetic” Se in Zn-64
Rb 4.94 mcg to compensate “magnetic” Rb in Zn-67
Sr 22.84 mg to compensate “magnetic” Sr in Zn-68
Zr 0.74 mcg to compensate “magnetic” Zr in Zn-70
2. For the enriched Zn-64 (99, 31% enrichment) compensatory additive:
Mn 2.3 mg dose of Mn-55
Zn 20 mg to compensate “magnetic” Zn in Mn-55
Se 122.60 mcg to compensate “magnetic” Se in Zn-64
Rb 0.06 mcg to compensate “magnetic” Rb in Zn-67
Sr 0.26 mcg to compensate “magnetic” Sr in Zn-68
Zr 0.01 mcg to compensate “magnetic” Zr in Zn-70

Only these compositions will provide a balance between \vec{Π}e and \vec{Π}m fields and healthy homeostasis in live cells of the human organism.

The role of chemical elements without known biological effect.

There are many chemical elements that are not only not essential but have no biological role at all. For example, Ac, Al, Sb, As, At, Ba, Bi, Cs, Gd, Ge, In, Ir, Nb, Pl, Pt, Rn, Rh, Rt, Sm, Sc, Ta, Tl, Tr, Tl, W, Y, Zr.

These chemical elements are not included in the structure of biomolecules; they do not bid with biomolecules; they do not participate in any chemical reactions. Why are they present and even accumulated in live cells of the human body? Is it just by chance that they come together with food and water in proportion to the content in nature?

To answer these questions, consider what may be required to support a balance between \vec{Π}e and \vec{Π}m fields in a live cell in connection with the chemical element calcium. There are five stable isotopes (40Ca, 42Ca, 43Ca, 44Ca and 46Ca), plus one isotope (48Ca) with such a long half-life that for all practical purposes it can be considered stable.

The intermediate reasoning is omitted as carried out in the previous examples and written down which chemical elements are necessary to compensate for the magnetic component of calcium isotopes:
Ca dose of Ca-40
Ti to compensate “magnetic” Ti in Ca-42
V to compensate “magnetic” V in Ca-43
Cr to compensate “magnetic” Cr in Ca-44
Fe to compensate “magnetic” Fe in Ca-46
Ni to compensate “magnetic” Ni in Ca-48

In the examples above the necessity to use Ga, Ge, and As is observed. If an example of a heavier element with a biological role is considered, then the need to use even rare earth elements to compensate for the magnetic components of the isotopes of this element will be seen. It means that in the absence of a source of the lightest isotopes of essential elements, live cells use the only available option to maintain a balance between \vec{Π}e and \vec{Π}m. In other words, chemical elements with the number of protons equal to the number of neutrons in each of the isotopes of the chemical elements present in the cell become part of the auto-oscillating system of electric and magnetic polarization potentials, thereby ensuring the stability of the entire system/cell. Thus, chemical elements whose biological role has not yet been clarified are directly involved in maintaining stability of live cells by supporting negative feedbacks, that is, homeostasis.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the appended claims. Thus, while only certain features of the invention have been illustrated and described, many modifications and changes will occur to those skilled in the art. It is therefore to be understood that the appended claims are intended to cover all such modifications and changes as fall within the true spirit of the invention.

1. A method to restore homeostasis in a subject in need thereof by establishing or rebuilding a homeostatic balance between electric and magnetic components of differential and integral characteristics of electromagnetic field in cells of said subject, comprising administering to said subject a therapeutically effective amount of a pharmaceutical composition comprising various essential chemical elements and additive chemical elements specific to each essential chemical element in specific quantities.

2.-20. (canceled)

21. A method of treating or preventing a degenerative disease in a subject in need thereof by establishing or rebuilding a homeostatic balance between electric and magnetic components of differential and integral characteristics of electromagnetic field in cells of said subject, comprising administering to said subject a therapeutically effective amount of a pharmaceutical composition comprising various essential chemical elements and additive elements specific to each essential element in specific quantities.

22. The method of claim 21, wherein each additive chemical element specific to each essential chemical element is chosen as a pair for each isotope of said additive chemical element such that number of protons in the additive chemical element is equal to number of neutrons in the corresponding isotope of the essential chemical element.

23. The method of claim 21, wherein the essential chemical elements and additive chemical elements have natural abundance of isotopes.

24. The method of claim 21, wherein each essential chemical element contains at least 80% of its lightest isotope and each additive chemical element has natural abundance of isotope.

25. The method of claim 21, wherein the essential chemical elements have natural abundance of isotopes and at least one additive chemical element contains at least 80% of its lightest isotope.

26. The method of claim 21, wherein the essential chemical elements contain at least 80% of its lightest isotope and each of the additive chemical elements contains at least 80% of its lightest isotope.

27. The method of claim 21, wherein a therapeutic dose of each additive chemical elements is chosen by calculating as a product between ratio of the daily consumption dose of the additive chemical element that compensates for the magnetic component of the lightest isotope of one of the essential chemical elements to the abundance of said isotope in nature and the abundance of each isotope of said essential chemical element.

28. The method of claim 21, wherein the pharmaceutical composition comprises the essential chemical element Fe and additive chemical elements Ni, Zn, Ga and Ge, and a therapeutic dose equal to or multiple of a sum of: 20 mg Fe+0.15 mg Ni+2.36 mg Zn+50 mcg Ga+7.2 mcg Ge.

29. The method of claim 21, wherein the pharmaceutical composition comprises the essential chemical element Ni and additive chemical elements Zn, Ge, As and Se, and a therapeutic dose equal to or multiple of a sum of: 0.15 mg Ni+20 mg Zn+7.7 mg Ge+335 mcg As+1.07 mg Se.

30. The method of claim 21, wherein the pharmaceutical composition comprises the essential chemical element Zn and additive chemical elements Se, Rb, Sr and Zr, and a therapeutic dose equal to or multiple of a sum of: 20 mg Zn+60 mcg Se+200 mcg Rb+22.84 mg Sr+0.74 mcg Zr.

31. The method of claim 21, wherein the pharmaceutical composition comprises the essential chemical element Mg and additive chemical elements Al and Si, and a therapeutic dose equal to or multiple of a sum of: 370 mg Mg+10 mg Al+1.39 mg Si.

32. The method of claim 21, wherein the pharmaceutical composition comprises the essential chemical element K and additive chemical elements Ca, Sc and Ti, and a therapeutic dose equal to or multiple of a sum of: 3500 mg K+2500 mg Ca+320 mcg Sc+169 mg Ti

33. The method of claim 21, wherein the pharmaceutical composition comprises the essential chemical element Mn and additive chemical elements Zn, Se, Rb, Sr, Zr and a therapeutic dose equal to or multiple of a sum of: 2.3 mg Mn+20 mg Zn+60 mcg Se+200 mcg Rb+22.84 mg Sr+0.74 mcg Zr.

34. The method of claim 21, wherein the pharmaceutical composition comprises the essential chemical element Fe enriched to 90% isotope Fe-54 and additive chemical elements Ni, Zn, Ga and Ge, and a therapeutic dose equal to or multiple of a sum of: 20 mg Fe-54+2.31 mg Ni+0.23 mg Zn+26 mg Ga+2.6 mg Ge.

35. The method of claim 21, wherein the pharmaceutical composition comprises the essential chemical element Zn enriched to 99.31% isotope Zn-64 and additive chemical elements Se, Rb, Sr and Zr, and a therapeutic dose equal to or multiple of a sum of: 20 mg Zn-64+122.6 mcg Se+200 mcg Rb+0.26 mg Sr+0.01 mcg Zr.

36. The method of claim 21, wherein the pharmaceutical composition comprises the essential chemical element Se enriched to 99.30% isotope Se-74 and additional elements Sr and Mo and therapeutic dose equal to or multiple of a sum of: 60 mcg Se-74+55.3 mg Zr+5.14 mcg Mo.

37. The method of claim 21, wherein the pharmaceutical composition comprises the essential chemical element Mo enriched to 92.0% isotope Mo-92 and additional element Sn and a therapeutic dose equal to or multiple of a sum of: 100 mcg Mo-92+20.27 mcg Sn.

38.-40. (canceled)

41. A method of achieving anti-aging effect in a subject in need thereof comprising establishing or rebuilding a homeostatic balance between electric and magnetic components of differential and integral characteristics of electromagnetic field in cells of said subject, by administering to said subject a therapeutically effective amount of a pharmaceutical composition comprising various essential chemical elements and additive elements specific to each essential element in specific quantities.

42.-60. (canceled)

61. A pharmaceutical composition comprising therapeutically effective amounts of various essential chemical elements and additive chemical elements specific to each essential chemical element in specific quantities for restoring homeostasis in a subject in need thereof, by establishing or rebuilding a homeostatic balance between electric and magnetic components of differential and integral characteristics of electromagnetic field in cells of said subject.
